(19) United States
(12) Patent Application Publication
(10) Pub. No.: US 2009/0068637 A1
(43) Pub. Date: Mar. 12, 2009

(54) MONOCLONAL ANTIBODIES BINDING TO AVIAN INFLUENZA VIRUS SUBTYPE H5 HAEMAGGLUTININ AND USE THEREOF

(76) Inventors: Ningshao Xia, Xiamen (CN); Yixin Chen, Xiamen (CN); Shengxiang Ge, Xiamen (CN); Wenxin Luo, Xiamen (CN); Jun Zhang, Xiamen (CN)

Correspondence Address: PERKINS COIE LLP, POST OFFICE BOX 1208, SEATTLE, WA, 98111-1208, US

(21) Appl. No.: 12/162,343

(22) PCT Filed: Jan. 26, 2007
(86) PCT No.: PCT/US07/02491

§ 371 Date: Jul. 25, 2008

(30) Foreign Application Priority Data
Jan. 26, 2006 (CN) 200610002312.1

(51) Int. Cl. C12Q 1/70; C07K 16/08; C12M 1/34; C07H 21/04

(52) U.S. Cl. 435/5; 530/388.3; 536/23.53; 435/287.2

(57) ABSTRACT

The present application provides monoclonal antibodies that specifically bind to the hemagglutinin of avian influenza virus subtype H5, as well as monoclonal antibodies capable of blocking at least 50% of the hemagglutinin binding activity of these monoclonal antibodies. Such antibodies are useful, for example, in the detection, diagnosis, prevention, and treatment of avian influenza virus. Also provided herein are hybridoma cell lines, isolated nucleic acid molecules, and short peptides related to the monoclonal antibodies provided herein, and pharmaceutical compositions and kits containing the monoclonal antibodies provided herein.

### RELATED APPLICATION

This application claims benefit from Chinese Patent Application No. 200610002312.1 filed on Jan. 26, 2006, which is incorporated by reference herein in its entirety.

### FIELD OF THE INVENTION

This application relates to monoclonal antibodies binding to avian influenza virus subtype H5 haemagglutinin (HA) and fragments thereof, their peptide sequences, cell lines producing such monoclonal antibodies, and methods of using the antibodies and fragments thereof for diagnostic and therapeutic purposes.

### BACKGROUND OF THE INVENTION

Since H5 avian influenza broke out first in a goose farm in Guangdong province of China in 1996 (Xu, X. et al., 1999, Virology), influenza outbreaks have been caused by another derived H5 virus in an avian farm in Hong Kong (April 1997) and in a market (November 1997). The direct transmission of the avian influenza virus from avian to human was the first such recorded transmission in human history. Eighteen people were finally diagnosed as being infected by the avian influenza virus and six of them died. Since 2003, successive outbreaks of H5 avian influenza have swept across the countries of the East and Southeast Asia. The World Health Organization (WHO) and flu experts predicted that subtype H5 avian influenza virus would be the most likely epidemic virus strain responsible for the next human flu outbreak. In early 2004, the highly pathogenic H5 avian influenza broke out successively in more than ten provinces in China, and animals including chickens, ducks, herons, tigers, and cats were reported to be infected by the H5 avian influenza virus in Hong Kong, Thailand, the Netherlands and other countries. Even worse were suspected incidents of human-to-human infection in Thailand and Malaysia. In 2005, cases of birds dying from infection with H5 avian influenza virus were successively reported in European countries including Romania, Russia, and Turkey, and experts believed that it was the migration of the migratory birds with virus that made the control of the further diffusion and transmission of the highly pathogenic H5 avian influenza more difficult. Specialists predicted that, spread by migratory birds, the subtype H5 of the avian influenza virus might further transmit to African countries through Eurasia and the Afro-Asia Land Bridge where sanitation conditions were filthy, which might provide chances and time for the subtype H5 of the avian influenza virus to recombine fully with other human influenza viruses. At that time, a brand new and deadly human influenza virus might appear, and it would be difficult to estimate the great loss of human life caused thereby. According to WHO's statistics, by Jan. 19, 2006, the human death toll in the world caused by infection with the H5N1 virus had gone up to 80, which brought a great challenge for global public health safety.

The latest research (Li, K. S. et al., 2004, Nature) indicates that water birds in Southern China (duck) were the main carriers and transmitters of subtype H5 of the avian influenza virus, and its outbreak was seasonal and accompanied with the evolution of bio-multiformity (multi-genotype). However, research on the molecular epidemiology indicated that about 30% of the infected ducks showed no symptoms and up to 10% of the infected chickens were prevalent and non-symptomatic carriers. These infected animals could infect human beings continuously, which would threaten human health enormously. Experts all agree that control of the spreading of the highly pathogenic H5 avian influenza virus in East Asia, Southeast Asia, and Europe can only be assured by early diagnosis, early isolation, early management, and early treatment to human.

It takes 4-5 days to diagnose avian influenza virus by the traditional viral isolation and serum diagnosis method, and most human and animal disease control laboratory systems lack Grade-3 biosafety laboratories. Thus, diagnosis of the H5 avian influenza outbreak in the countries and regions of Southeast Asia was obviously delayed. Frequently, no final diagnosis was reported after a large number of chickens had died or been killed. This situation made it difficult to control the virus outbreak. In addition, no-symptom carriers among some birds (especially water birds, such as ducks) posed great problems, and there has not been an effective test facility in the present quarantine system. This resulted in the virus breaking out repeatedly in many countries and regions.

The H5 avian influenza virus (among which Goose/Guangdong/1/96 was the representative strain) belongs to a group of highly pathogenic viruses, and are fatal to all common domestic avians. However, the antigenicity of the HA cannot be completely obtained through genetic engineering methods. Many world famous laboratories have attempted but failed to prepare monoclonal antibody targeting on the virus. At present, virus antigenicity analysis has to adopt the monoclonal antibody prepared by A/chicken/Pennsylvania/1370/83 (H5N2) and A/chicken/Pennsylvania/8125/83 (H5N2), neither of which meets the requirements of specificity and reactivity for the diagnostic reagent.

Therefore, a method for convenient real time diagnosis is urgently required. This would allow patients from the first cross-species infected generation to be isolated and treated, resulting in the prevention of person to person infection and interruption of the transmission chain before the virus has adapted to human beings. Finally, the threat of the virus to cause a widely spread human influenza can be fundamentally eliminated.

In China, research on detecting anti-subtype H5 of the avian influenza virus has been reported. Qin et al. from College of Animal Husbandry and Veterinary Medicine, Yangzhou University, (Qin, Aijian et al., Journal of Chinese Prevention Veterinary Medicine, 2003, No. 3) prepared HA specific monoclonal antibodies for the avian influenza viruses of subtype H5 and subtype H9, and it was confirmed that with these monoclonal antibodies, the corresponding avian influenza virus could be quickly detected within 24 hours by indirect immunofluorescence assay. It was further clinically confirmed on Dec. 10, 2005 that the detection time for the highly pathogenic subtype H5 of the avian influenza virus could be shortened to 4 hours, which test was conducted by the Beijing Office for Entry-Exit Inspection and Quarantine with a rapid fluorescent RT-PCR. Guo Yuanji mentioned that micro-neutralization experiment or ELISA with high specificity was needed for detecting the antibody for the virus strain of subtype H5 (Guo Yuanji, “Human Avian Influenza Research Present Situation,” Chinese Journal of Experimental and Clinical Virology, 2004, No. 3), however, no research has been reported on the detection of the subtype H5 by ELISA.

The detection of H5N1 antibody by ELISA has been reported outside China. Rowe et al. reported the use of a recombinant HA protein as the antigen covering to detect the H5N1 antibody by indirect ELISA, and the sensitivity of the ELISA was 80% and specificity 62% (Rowe, T. et al., J. Clin. Microbiol., April 1999, 37 (4): 937-43). However, this research did not aim directly at the specific monoclonal antibody of the HA gene of the subtype H5N1. Zhou et al. (Zhou, E. M. et al., Avian Dis., 1998, 42 (4): 757-61) and Shafer et al (Shafer, A. L., et al., Avian Dis., 1998, 42 (1): 28-34) detected an antibody for an anti-core protein by competitive ELISA. However, the subjects were all antibodies for the NP proteins of all subtype H11-H116 of type A avian influenza, and the subtype could not be confirmed. Lu reported a method for detecting avian influenza virus (AIV) by Dot-ELISA on the basis of a monoclonal antibody. The method detected the AIV antigen directly with no cross-reaction to other avian viruses (Lu, H., Avian Dis., 2003, 47 (2): 361-9). Although Sala et al. established an ELISA based on a monoclonal antibody of the specific surface glycoprotein of subtype H7, the subtype differed from H5 and the monoclonal antibody was specific to the surface glycoprotein (Sala G, Cordioli P, Moreno-Martin et al., Avian Dis. 2003, 47 (3 Suppl): 1057-9), rather than specific to the HA gene.

Unfortunately, most of the monoclonal antibodies used in immunological diagnosis of the avian influenza virus aim directly at the core protein (NP protein), and thus are not capable of distinguishing between type A subtypes. Type A influenza virus actually includes subtypes H1-H16 with 16 subtypes in total, among which most subtypes have no pathogenicity or only low pathogenicity and only subtype H5 is the most harmful avian influenza virus with high pathogenicity. Thus the available technologies are far away from meeting the demands of clinic detections.

The purpose of this invention is to overcome the shortcomings of the available immuno-detection technologies for the avian influenza virus. The monoclonal antibody adopted in this invention aims directly at the HA protein of subtype H5, allowing for specific detection of the highly pathogenic subtype H5 of the avian influenza virus.

### SUMMARY OF THE INVENTION

The present invention provides monoclonal antibodies that specifically bind to the hemagglutinin of avian influenza virus subtype H5, as well as monoclonal antibodies capable of blocking at least 50% of the hemagglutinin binding activity of these antibodies. The present invention also provides hybridoma cell lines, isolated nucleic acid molecules, and short peptides related thereto, as well as a pharmaceutical composition, detection devices, and kits containing the monoclonal antibodies. The present invention also provides methods of detecting, diagnosing, preventing, and treating avian influenza virus, particularly subtype H5 of the avian influenza virus, using the monoclonal antibodies provided herein.

### BRIEF DESCRIPTION OF THE FIGURES

FIG. 1 shows the detection results of the HA antigen detection kit for subtype H5 of the avian influenza virus by the gold mark method, in which “a” indicates positive when two red lines appear; “b” indicates negative when only one and the quality control line appears; “c” indicates invalid test result when no red line appears.

FIG. 2 shows the detection results of the anti-HA antibody detection kit for subtype H5 of the avian influenza virus by the gold mark method, in which “a” indicates positive when only one and the quality control line appears; “b” indicates negative when two red lines appear; “c” indicates invalid test result when no red line appears.

FIG. 3 shows the detection results of the HA antigen Dot-ELISA detection kit for subtype H5 of the avian influenza virus, in which “+” indicates positive when color appears in the oval area; “−” indicates negative when no color appears in the oval area.

FIG. 4 shows schematic diagrams of 6 expression plasmids for chimeric antibodies: pcDNA3.1-Ak8H5, pcDNA3.1-AH8H5, pcDNA3.1-Ak10F7, pcDNA3.1-AH10F7, pcDNA3.1-Ak4D1, and pcDNA3.1-AH4D1.

FIG. 5 shows the results of HA coagulation inhibition test of three types of chimeric antibodies with the virus strain Ck/HK/Yu22/02. Rows 1, 2 and 3: PBS control; Rows 4 and 5: 10F7 cAb; Row 6: 10F7 mAb; Rows 7 and 8: 4D1 cAb; Row 9: 4D1 mAb; Rows 10 and 11: 8H5 cAb; Row 12: 8H5 mAb.

FIG. 6 shows the results of immuno-fluorescent assay of chimeric antibodies with cells expressing H5 hemagglutinin. A. cAb 4D1 (DAPI); B. cAb 4D1 (FITC); C. cAb 10F7 (DAPI); D. cAb 10F7 (FITC); E. anti-HBV cAb (DAPI); F. anti-HBV cAb (FITC).

FIG. 7 is a histogram of the OD (450/620) values of ELISA test for the bacteriophage peptides phagotope.

FIG. 8 shows schematic diagrams of plasmid maps of pTO-T7 and pTO-T7-239-123.

FIG. 9 shows schematic diagrams of plasmid maps of pTO-T7 and pTO-T7-239-125.

FIG. 10 shows the SDS-PAGE picture of purified fusion protein (or recombinant protein) 239-123. Lane 1: Protein molecular weight markers; Lane 2: Whole bacterial lysate of E. coli expressing 239-123; Lane 3: Supernatant obtained by centrifugation of the whole bacterial lysate; Lane 4: 239-123 in buffer I; Lane 5: Purified fusion protein 239-123 in 2M Urea; Lane 6: Purified fusion protein 239-123 in 4M Urea; Lane 7: Purified fusion protein 239-123 in 8M Urea.

FIG. 11 shows the SDS-PAGE picture of purified fusion protein 239-125. Lane 1: Protein molecular weight markers; Lane 2: Whole bacterial lysate of E. coli expressing 239-125; Lane 3: Supernatant obtained by centrifugation of the whole bacterial lysate; Lane 4: 239-125 in buffer I; Lane 5: Purified fusion protein 239-125 in 2M Urea; Lane 6: Purified fusion protein 239-125 in 4M Urea; Lane 7: Purified fusion protein 239-125 in 8M Urea.

FIG. 12 indicates the specific affinity of fusion protein 239-123 with a histogram of the color intensities (shown as OD (450/620) values) of ELISA test of fusion protein 239-123 binding to various antibody strains as labeled on the horizontal axis.

FIG. 13 indicates the specific affinity of the fusion protein 239-125: it is a histogram of the color intensities (shown as OD (450/620) values) of ELISA test of fusion protein 239-125 binding to various antibody strains as labeled on the horizontal axis.

FIG. 14 shows the color intensities (shown as OD (450/620) values) of the ELISA test of fusion protein 239-123 binding to 8H5 mAb (triangle dotted line) or 8C11 mAb (square dotted line) at a series of dilutions of the mAb.

FIG. 15 shows schematic diagrams of plasmids pC149-mut and pC149-mut-123.

FIG. 16 shows schematic diagrams of plasmids pC149-mut and pC149-mut-125.

FIG. 17 shows the SDS-PAGE picture of whole cell lysate of small scale expressed “recombinant” proteins. Lane 1: D123; Lane 2: T123; Lane 3: F123; Lane 4: Q123; Lane 5: D125; Lane 6: T125; Lane 7: F125; Lane 8: Q125.

FIG. 18 shows the SDS-PAGE picture of purified recombinant proteins. Lane 1: D123; Lane 2: T123; Lane 3: F123; Lane 4: Q123; Lane 5: D125; Lane 6: T125; Lane 7: F125; Lane 8: Q125.

FIG. 19 are electron microscope pictures showing virus-like particles assembled from recombinant proteins of HBV cAg fragment and antibody-binding peptides.

FIG. 20 a histogram of the color intensities (shown as OD (450/620) values) of ELISA test of the binding affinity and reactivity between fusion proteins HBc-123/125 and 8H5 mAb.

FIG. 21 a histogram of the color intensities (shown as OD (450/620) values) of ELISA test of the binding affinity and reaction between fusion proteins HBc-Q123 or HBc-D125 with various mAb. The results showed that the fusion proteins HBc-Q123 and HBc-D125 bound specifically to 8H5 mAb.

FIG. 22 shows the increase (ascending curve) in immune mouse serum antibody titer of mice immuned by fusion protein HBc-123 from 0 to 4 weeks. The antibody titer was detected by ELISA.

FIG. 23 shows the increase (ascending curve) in immune mouse serum antibody titer of mice immuned by fusion protein HBc-125 from 0 to 4 weeks. The antibody titer was detected by ELISA.

FIG. 24 shows immuno-fluorescence pictures of reaction between immune mouse serum and HA protein expressed in SF21 cells.

FIG. 25 contains electron microscope pictures showing virus-like particles assembled by recombinant protein HBc-122, HBc-124, HBc-128, and HBc-129.

FIG. 26 a histogram of the color intensities (shown as OD (450/620) values) of ELISA test of the binding affinity between fusion proteins HBc-122, HBc-124, HBc-128, and HBc-129 to various mAb. The results showed that the fusion proteins HBc-122, HBc-124, HBc-128 and HBc-129 bound specifically to 8H5 mAb.

FIG. 27 shows the results of competition binding of and virus like particles assembled from fusion proteins of 12aa 12 VLP peptides and H5N1 virus to an Enzyme-labeled 8H5 mAb. The vertical axis is color intensities (shown as OD (450/620) values). The horizontal axis is various virus like particles and a PBS control used in the tests.

### DETAILED DESCRIPTION OF THE INVENTION

### Definitions

The term “hemagglutinin” as used herein refers to an envelope glycoprotein of the avian influenza virus. Hemagglutinins mediate adsorption and penetration of the influenza virus into a host cell. Avian influenza virus hemagglutinin proteins exhibit sixteen different serological subtypes, HA 1 to HA 16, associated with the sixteen viral subtypes H1-H16 respectively.

The term “antibody” as used herein refers to any immunoglobulin, including monoclonal antibodies, polyclonal antibodies, multispecific or bispecific antibodies, that bind to a specific antigen. A complete antibody comprises two heavy chains and two light chains. Each heavy chain consists of a variable region and a first, second, and third constant region, while each light chain consists of a variable region and a constant region. The antibody has a “Y” shape, with the stem of the Y consisting of the second and third constant regions of two heavy chains bound together via disulfide bonding. Each arm of the Y consists of the variable region and first constant region of a single heavy chain bound to the variable and constant regions of a single light chain. The variable regions of the light and heavy chains are responsible for antigen binding. The variable region in both chains generally contains three highly variable loops called the complementarity determining regions (CDRs) (light (L) chain CDRs including LCDR1, LCDR2, and LCDR3, heavy (H) chain CDRs including HCDR1, HCDR2, HCDR3) (as defined by Kabat, et al., Sequences of Proteins of Immunological Interest, Fifth Edition (1991), vols. 1-3, NIH Publication 91-3242, Bethesda Md.). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The major classes of antibodies are IgA, IgD, IgE, IgG, and IgM, with several of these classes divided into subclasses such as IgG1, IgG2, IgG3, IgG4, IgA1, or IgA2.

In addition to an intact immunoglobulin, the term “antibody” as used herein further refers to an immunoglobulin fragment thereof (i.e., at least one immunologically active portion of an immunoglobulin molecule), such as a Fab, Fab′, F(ab′)₂, Fv fragment, a single-chain antibody molecule, a multispecific antibody formed from any fragment of an immunoglobulin molecule comprising one or more CDRs. In addition, an antibody as used herein may comprise one or more CDRs from a particular human immunoglobulin grafted to a framework region from one or more different human immunoglobulins.

“Fab” with regards to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂ refers to a dimer of Fab′.

“Fc” with regards to an antibody refers to that portion of the antibody consisting of the second and third constant regions of a first heavy chain bound to the second and third constant regions of a second heavy chain via disulfide bonding. The Fc portion of the antibody is responsible for various effector functions but does not function in antigen binding.

“Fv” with regards to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Houston 1988).

“Single-chain Fv-Fc antibody” or “scFv-Fc” refers to an engineered antibody consisting of a scFv connected to the Fc region of an antibody.

The term “epitope” as used herein refers to the group of atoms and/or amino acids on an antigen molecule to which an antibody binds.

The term “monoclonal antibody” or “MAb” or “mAb” as used herein refers to an antibody or a fragment thereof obtained from a population of substantially homogeneous antibodies, i.e., the individual antibodies comprising the population are identical except for possible naturally occurring mutations that may be present in minor amounts. Monoclonal antibodies are highly specific, being directed against a single epitope on the antigen. Monoclonal antibodies are in contrast to polyclonal antibodies which typically include different antibodies directed against different epitopes on the antigens. Although monoclonal antibodies are traditionally derived from hybridomas, the monoclonal antibodies of the present invention are not limited by their production method. For example, the monoclonal antibodies of the present invention may be made by the hybridoma method first described by Kohler et al., Nature, 256:495 (1975), or may be made by recombinant DNA methods (see, e.g., U.S. Pat. No. 4,816,567).

The term “chimeric antibody” as used herein refers to an antibody in which a portion of the heavy and/or light chain is identical with or homologous to corresponding sequences in antibodies derived from a particular species or belonging to a particular antibody class or subclass, while the remainder of the heavy and/or light chain is identical with or homologous to corresponding sequences in antibodies derived from another species or belonging to another antibody class or subclass, as well as fragments of such an antibody, so long as such fragments exhibit the desired antigen-binding activity (U.S. Pat. No. 4,816,567 to Cabilly et al.; Morrison et al., Proc. Natl. Acad. Sci. USA, 81:6851 6855 (1984)).

The term “humanized antibody” used herein refers to an antibody or fragments thereof which are human immunoglobulins (recipient antibody) in which residues from part or all of a CDR of the recipient are replaced by residues from a CDR of a non-human species (donor antibody) such as mouse, rat or rabbit having the desired specificity, affinity, and capacity. In some instances, FR residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin Fc region, typically that of a human immunoglobulin. For further details, see Jones et al., Nature, 321:522 525 (1986); Reichmann et al., Nature, 332:323 329 (1988); Presta, Curr. Op. Struct. Biol., 2:593 596 (1992), and Clark, Immunol. Today 21: 397 402 (2000).

The term “isolated” as used herein means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. “Isolated” as used herein does not exclude artificial or synthetic mixtures with other compounds or materials, or the presence of impurities that do not interfere with activity.

The term “vector” as used herein refers to a nucleic acid vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The term “host cell” as used herein refers to a cell into which a vector has been introduced. A host cell may be selected from a variety of cell types, including for example bacterial cells such as E. coli or B. subtilis cells, fungal cells such as yeast cells or Aspergillus cells, insect cells such as Drosophila S2 or Spodoptera Sf9 cells, or animal cells such as fibroblasts, CHO cells, COS cells, NSO cells, HeLa cells, BHK cells, HEK 293 cells, or human cells.

The term “neutralizing antibody” as used herein refers to an antibody or fragments thereof which is able to eliminate or significantly reduce the virulency of a target viral antigen to which it binds.

The term “percent (%) sequence identity” with respect to the nucleic acid or polypeptide sequences referred to herein is defined as the percentage of nucleic acid or amino acid residues in a candidate sequence that are identical with the nucleic acid or amino acid residues, respectively, in a sequence, after aligning the sequences and introducing gaps, if necessary, to achieve the maximum percent sequence identity, and not considering any conservative substitutions as part of the sequence identity. Alignment for purposes of determining percent nucleic acid or amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The term “specifically binds” as used herein refers to a non-random binding reaction between two molecules, such as for example between an antibody and an antigen against which the antibody is raised. As used herein, an antibody that specifically binds a first antigen may exhibit no detectable binding affinity or low level binding affinity with a second antigen. In certain embodiments, an antibody that specifically binds an antigen binds the antigen with a binding affinity (K_{D}) of ≦10⁻⁵ M (e.g., 10⁻⁶ M, 10⁻⁷ M, 10⁻⁸ M, 10⁻⁹ M, 10⁻¹⁰ M, etc.). K_{D}, which as used herein refers to the ratio of the dissociation rate to the association rate (k_{off}/kₒₙ), may be determined using methods known in the art.

Antibodies

The present invention provides monoclonal antibodies that specifically bind to subtype H5 avian influenza virus. One aspect of this invention relates to monoclonal antibodies that can bind specifically to the hemagglutinin of subtype H5 avian influenza virus and various antigen-binding fragments of such monoclonal antibodies.

The present invention provides anti-H5 monoclonal antibodies that are produced by mice hybridoma cell strains 8H5, 3C8, 10F7, 4D1, 3G4 and 2F2. These monoclonal antibodies are named after the hybridoma cell strains that produce them. Thus the anti-H5 monoclonal antibodies that are produced by mice hybridoma cell strains 8H5, 3C8, 10F7, 4D1, 3G4, and 2F2, respectively, are named monoclonal antibodies 8H5, 3C8, 10F7, 4D1, 3G4, and 2F2, respectively. Monoclonal antibodies 8H5, 3C8, 10F7, 4D1, 3G4, and 2F2 specifically bind to the hemagglutinin of subtype H5 avian influenza virus. The mice hybridoma cell strains 8H5, 3C8, 10F7, 4D1, 3G4, and 2F2 were deposited in China Center for Typical Culture Collection (CCTCC, Wuhan University, Wuhan, China) on Jan. 17, 2006 with deposit numbers of CCTCC-C200607 (hybridoma cell strain 8H5), CCTCC-C200605 (hybridoma cell strain 3C8), CCTCC-C200608 (hybridoma cell strain 10F7), CCTCC-C200606 (hybridoma cell strain 4D1), CCTCC-C200604 (hybridoma cell strain 3G4) and CCTCC-C200424 (hybridoma cell strain 2F2).

The present invention also provides monoclonal antibodies that block the binding of monoclonal antibodies 8H5, 3C8, 10F7, 4D1, 3G4, or 2F2 to the hemagglutinin of subtype H5 avian influenza virus. Such blocking monoclonal antibodies may bind to the same epitopes on the hemagglutinin that are recognized by monoclonal antibodies 8H5, 3C8, 10F7, 4D1, 3G4, or 2F2. Alternatively, those blocking monoclonal antibodies may bind to epitopes that overlap sterically with the epitopes recognized by monoclonal antibodies 8H5, 3C8, 10F7, 4D1, 3G4, or 2F2. These blocking monoclonal antibodies can reduce the binding of monoclonal antibodies 8H5, 3C8, 10F7, 4D1, 3G4, or 2F2 to the hemagglutinin of subtype H5 avian influenza virus by at least about 50%. Alternatively, they may reduce binding by at least about 60%, preferably at least about 70%, more preferably at least about 75%, more preferably at least about 80%, more preferably at least about 85%, even more preferably at least about 90%, even more preferably at least about 95%, most preferably at least about 99%.

The ability of a test monoclonal antibody to reduce the binding of a known monoclonal antibody to the H5 hemagglutinin may be measured by a routine competition assay such as that described in Antibodies: A Laboratory Manual, Cold Spring Harbor Laboratory, Ed Harlow and David Lane (1988). For example, such an assay could be performed by pre-coating a microtiter plate with antigens, incubating the pre-coated plates with serial dilutions of the unlabeled test antibodies admixed with a selected concentration of the labeled known antibodies, washing the incubation mixture, and detecting and measuring the amount of the known antibodies bound to the plates at the various dilutions of the test antibodies. The stronger the test antibodies compete with the known antibodies for binding to the antigens, the more the binding of the known antibodies to the antigens would be reduced. Usually, the antigens are pre-coated on a 96-well plate, and the ability of unlabeled antibodies to block the binding of labeled antibodies is measured using radioactive or enzyme labels.

Monoclonal antibodies may be generated by the hybridoma method first described by Kohler et al., Nature, 256: 495 (1975). In the hybridoma method, a mouse or other appropriate host animal is immunized by one or more injections of an immunizing agent and, if desired, an adjuvant. Typically, the immunizing agent and/or adjuvant will be injected in the host animal by multiple subcutaneous or intraperitoneal injections. It may be useful to conjugate the immunizing agent to a protein known to be immunogenic in the host animal being immunized, such as serum albumin, or soybean trypsin inhibitor. Examples of adjuvants which may be employed include Freund's complete adjuvant and MPL-TDM. After immunization, the host animal makes lymphocytes that produce or are capable of producing antibodies that will specifically bind to the antigen used for immunization. Alternatively, lymphocytes may be immunized in vitro. Desired lymphocytes are collected and fused with myeloma cells using a suitable fusing agent, such as polyethylene glycol, to form a hybridoma cell (Goding, Monoclonal Antibodies: Principles and Practice, pp. 59-103, Academic Press, 1996).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and MC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63, Marcel Dekker, Inc., New York, 1987).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson et al., Anal. Biochem., 107: 220 (1980).

After hybridoma cells are identified that produce antibodies of the desired specificity, affinity, and/or activity, the cells may be subcloned by limiting dilution procedures and grown by standard methods (Goding, Monoclonal Antibodies: Principles and Practice, pp. 59-103, Academic Press, 1996). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies of the invention may also be made by conventional genetic engineering methods. DNA molecules encoding the heavy and light chains of the monoclonal antibodies may be isolated from the hybridoma cells, for example through PCR using oligonucleotide probes that are capable of binding specifically to genes encoding the heavy and light chains of the monoclonal antibodies. Then the DNA molecules are inserted into expression vectors. The expression vectors are transfected into host cells such as E. coli cells, simian COS cells, Chinese hamster ovary (CHO) cells, or myeloma cells that do not otherwise produce immunoglobulin protein. The host cells are cultured under conditions suitable for the expression of the antibodies.

The antibodies of the invention can bind to the H5 hemagglutinin with high specificity and affinity. The antibodies shall have low cross-reactivity with other subtypes of hemagglutinin, preferably no cross-reactivity with other subtypes of hemagglutinins. In one aspect, the invention provides antibodies that bind to H5 hemagglutinin with a K_{D} value of less than 1×10⁻⁵M. Preferably, the K_{D} value is less than 1×10⁻⁶M. More preferably, the K_{D} value is less than 1×10⁻⁷M. Most preferably, the K_{D} value is less than 1×10⁻⁸M.

The antibodies of the invention may contain the conventional “Y” shape structure comprised of two heavy chains and two light chains. In addition, the antibodies may also be the Fab fragment, the Fab′ fragment, the F(ab)₂ fragment or the Fv fragment, or another partial piece of the conventional “Y” shaped structure that maintains binding affinity to the hemagglutinin The binding affinity of the fragments to hemagglutinin may be higher or lower than that of the conventional “Y” shaped antibodies.

The antibody fragments may be generated via proteolytic digestion of intact antibodies (see, e.g., Morimoto et al., J. Biochem. Biophys. Methods, 24:107-117, (1992) and Brennan et al., Science, 229:81 (1985)). Additionally, these fragments can also be produced directly by recombinant host cells (reviewed in Hudson, Curr. Opin. Immunol., 11: 548-557 (1999); Little et al., Immunol. Today, 21: 364-370 (2000)). For example, Fab′ fragments can be directly recovered from E. coli and chemically coupled to form F(ab′)₂ fragments (Carter et al., Bio/Technology, 10:163 167 (1992)) In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to a person with ordinary skill in the art.

Antibody Nucleic Acid Sequences

The present invention provides isolated nucleic acid molecules encoding antibodies or fragments thereof that specifically bind to H5 hemagglutinin. Nucleic acid molecules encoding the antibodies can be isolated from hybridoma cells. The nucleic acid sequences of the molecules can be determined using routine techniques known to a person with ordinary skill in the art. Nucleic acid molecules of the invention can also be prepared using conventional genetic engineering techniques as well as chemical synthesis. In one aspect, the present invention provides an isolated nucleic acid molecule encoding the variable region of the heavy chain of an anti-H5 (HA) antibody or a portion of the nucleic acid molecule. In another aspect, the present invention provides an isolated nucleic acid molecule encoding the variable region of the light chain of an anti-H5 (HA) antibody or a portion of the nucleic acid molecule. In another aspect, the present invention provides an isolated nucleic acid molecule encoding the CDRs of the antibody heavy chain or light chain variable regions.

In one aspect, the present invention provides isolated nucleic acid molecules encoding the variable regions of the heavy chain and light chain of monoclonal antibodies 8H5, 3C8, 10F7, 4D1, 3G4, and 2F2. The nucleic acid sequences encoding the heavy chain variable regions (VH, Vh) of monoclonal antibodies 8H5, 3C8, 10F7, 4D1, 3G4, and 2F2 are set forth in SEQ ID NO: 1, SEQ ID NO: 5, SEQ ID NO: 9, SEQ ID NO: 16, SEQ ID NO:20 and SEQ ID NO: 24, respectively. The nucleic acid sequences encoding the light chain variable regions (VK, Vk) of monoclonal antibodies 8H5, 3C8, 10F7, 4D1, and 2F2 are set forth in SEQ ID NO: 3, SEQ ID NO: 7, SEQ ID NO: 11, SEQ ID NO: 18, SEQ ID NO: 26, respectively. The present invention also includes degenerative analogs of the nucleic acid molecules encoding the variable regions of the heavy chain and light chain of monoclonal antibodies 8H5, 3C8, 10F7, 4D1, 3G4 and 2F2.

In another aspect, the present invention provides isolated nucleic acid variants that share sequence identity with the nucleic acid sequences of SEQ ID NO: 1, SEQ ID NO: 3, SEQ ID NO: 5, SEQ ID NO: 7, SEQ ID NO: 9, SEQ ID NO: 11, SEQ ID NO: 16, SEQ ID NO:18, SEQ ID NO:20, SEQ ID NO:24 or SEQ ID NO:26. In one embodiment, the nucleic acid variants share at least 70% sequence identity, preferably at least 75% sequence identity, more preferably at least 80% sequence identity, more preferably at least 85% sequence identity, more preferably at least 90% sequence identity, most preferably at least 95% sequence identity, to the sequences of SEQ ID NO: 1, SEQ ID NO: 3, SEQ ID NO: 5, SEQ ID NO: 7, SEQ ID NO: 9, SEQ ID NO: 11, SEQ ID NO:16, SEQ ID NO:18, SEQ ID NO:20, SEQ ID NO:24 or SEQ ID NO:26.

The present invention also provides isolated nucleic acid molecules encoding antibody fragments that are still capable of specifically binding to subtype H5 of avian influenza virus.

The present invention further provides isolated nucleic acid molecules encoding an antibody heavy chain variable region comprising the amino acid sequence set forth in SEQ ID NOs: 28-30, SEQ ID NOs: 34-36, SEQ ID NOs: 40-42, SEQ ID NOs: 46-48; SEQ ID NOs: 52-54, or SEQ ID NOs: 58-60. The present invention provides isolated nucleic acid molecules encoding an antibody light chain variable region comprising the amino acid sequence set forth in SEQ ID NOs: 31-33, SEQ ID NOs: 37-39, SEQ ID NOs: 43-45, SEQ ID NOs: 49-51, or SEQ ID NOs: 61-63.

The present invention provides recombinant expressing vectors comprising the isolated nucleic acid molecules of the invention. It also provides host cells transformed with the nucleic acid molecules. Furthermore, the present invention provides a method of producing antibodies of the invention comprising culturing the host cells under conditions wherein the nucleic acid molecules are expressed to produce the antibodies and isolating the antibodies from the host cells.

Antibody Polypeptide Sequences

The amino acid sequences of the variable regions of the heavy chain and light chain of monoclonal antibodies 8H5, 3C8, 10F7, 4D1, 3G4 and 2F2 have been deduced from their respective nucleic acid sequences. The amino acid sequences of the heavy chain variable regions of monoclonal antibodies 8H5, 3C8, 10F7, 4D1, 3G4 and 2F2 are set forth in SEQ ID NO:2, SEQ ID NO:6, SEQ ID NO: 10, SEQ ID NO:17, SEQ ID NO:21, and SEQ ID NO:25, respectively. The amino acid sequences of the light chain variable regions of monoclonal antibodies 8H5, 3C8, 10F7, 4D1, and 2F2 are set forth in SEQ ID NO:4, SEQ ID NO:8, SEQ ID NO:12, SEQ ID NO:19, and SEQ ID NO:27 respectively. In one aspect, the present invention provides anti-H5 antibodies comprising a heavy chain variable region comprising the amino acid sequences as set forth in SEQ ID NO:2, SEQ ID NO:6, SEQ ID NO: 10, SEQ ID NO:17, SEQ ID NO:21, or SEQ ID NO:25. In another aspect, the present invention provides anti-H5 antibodies comprising a light chain variable region comprising the amino acid sequences as set forth in SEQ ID NO:4, SEQ ID NO:8, SEQ ID NO:12, SEQ ID NO:19, or SEQ ID NO:27.

In another aspect, the present invention provides an antibody heavy chain comprising a variable region having at least 70% sequence identity, preferably at least 75% sequence identity, more preferably at least 80% sequence identity, more preferably at least 85% sequence identity, more preferably at least 90% sequence identity, most preferably at least 95% sequence identity to the amino acid sequences set forth in SEQ ID NO:2, SEQ ID NO:6, SEQ ID NO: 10, SEQ ID NO:17, SEQ ID NO:21, or SEQ ID NO:25.

In another aspect, the present invention provides an antibody light chain comprising a variable region having at least 70% sequence identity, preferably at least 75% sequence identity, more preferably at least 80% sequence identity, more preferably at least 85% sequence identity, more preferably at least 90% sequence identity, most preferably at least 95% sequence identity to the amino acid sequences set forth in SEQ ID NO:4, SEQ ID NO:8, SEQ ID NO: 12, SEQ ID NO: 19, or SEQ ID NO:27.

The amino acid sequences of the CDRs of the variable regions of the heavy chain and light chain of monoclonal antibodies 8H5, 3C8, 10F7, 4D1, 3G4, and 2F2 have also been determined as follows:

The amino acid sequences of CDR1, CDR2 and CDR3 of the heavy chain of monoclonal antibody 8H5 are set forth in SEQ ID Nos:28-30, respectively. The amino acid sequences of CDR1, CDR2 and CDR3 of the light chain of monoclonal antibody 8H5 are set forth in SEQ ID Nos:31-33, respectively.

The amino acid sequences of CDR1, CDR2 and CDR3 of the heavy chain of monoclonal antibody 3C8 are set forth in SEQ ID Nos:34-36, respectively. The amino acid sequences of CDR1, CDR2 and CDR3 of the light chain of monoclonal antibody 3C8 are set forth in SEQ ID Nos:37-39, respectively.

The amino acid sequences of CDR1, CDR2 and CDR3 of the heavy chain of monoclonal antibody 10F7 are set forth in SEQ ID Nos:40-42, respectively. The amino acid sequences of CDR1, CDR2 and CDR3 of the light chain of monoclonal antibody 10F7 are set forth in SEQ ID Nos:43-45, respectively.

The amino acid sequences of CDR1, CDR2 and CDR3 of the heavy chain of monoclonal antibody 4D1 are set forth in SEQ ID Nos:46-48, respectively. The amino acid sequences of CDR1, CDR2 and CDR3 of the light chain of monoclonal antibody 4D1 are set forth in SEQ ID Nos:49-51, respectively.

The amino acid sequences of CDR1, CDR2 and CDR3 of the heavy chain of monoclonal antibody 3G4 are set forth in SEQ ID Nos:52-54, respectively.

The amino acid sequences of CDR1, CDR2 and CDR3 of the heavy chain of monoclonal antibody 2F2 are set forth in SEQ ID Nos:58-60, respectively. The amino acid sequences of CDR1, CDR2 and CDR3 of the light chain of monoclonal antibody 2F2 are set forth in SEQ ID Nos:61-63, respectively.

In another aspect, the present invention provides an anti-H5 monoclonal antibody heavy chain or a fragment thereof, comprising the following CDRs: (i) one or more CDRs selected from SEQ ID NOs: 28-30; (ii) one or more CDRs selected from SEQ ID NOs: 34-36; (iii) one or more CDRs selected from SEQ ID NOs: 40-42; (iv) one or more CDRs selected from SEQ ID NOs: 46-48; (v) one or more CDRs selected from SEQ ID NOs: 52-54; or (vi) one or more CDRs selected from SEQ ID NOs: 58-60. In one embodiment, the anti-H5 monoclonal antibody heavy chain or a fragment thereof comprises three CDRs having the amino acid sequences set forth in SEQ ID NOs: 28-30, respectively. In another embodiment, the anti-H5 monoclonal antibody heavy chain or a fragment thereof comprises three CDRs having the amino acid sequences set forth in SEQ ID NOs: 34-36, respectively. In another embodiment, the anti-H5 monoclonal antibody heavy chain or a fragment thereof comprises three CDRs having the amino acid sequences set forth in SEQ ID NOs: 40-42. In another embodiment, the anti-H5 monoclonal antibody heavy chain or a fragment thereof comprises three CDRs having the amino acid sequences set forth in SEQ ID NOs: 46-48. In another embodiment, the anti-H5 monoclonal antibody heavy chain or a fragment thereof comprises three CDRs having the amino acid sequences set forth in SEQ ID NOs: 52-54. In another embodiment, the anti-H5 monoclonal antibody heavy chain or a fragment thereof comprises three CDRs having the amino acid sequences set forth in SEQ ID NOs: 58-60.

In another aspect, the CDRs contained in the anti-H5 monoclonal antibody heavy chains or fragments thereof of the present invention may include one or more amino acid substitution, addition and/or deletion from the amino acid sequences set forth in SEQ ID NOs: 28-30, 34-36, 40-42, 46-48, 52-54, or 58-60. Preferably, the amino acid substitution, addition and/or deletion occur at no more than three amino acid positions. More preferably, the amino acid substitution, addition and/or deletion occur at no more than two amino acid positions. Most preferably, the amino acid substitution, addition and/or deletion occur at no more than one amino acid position.

In another aspect, the present invention provides an anti-H5 monoclonal antibody light chain or a fragment thereof, comprising the following CDRs: (i) one or more CDRs selected from SEQ ID NOs: 31-33; (ii) one or more CDRs selected from SEQ ID NOs: 37-39; (iii) one or more CDRs selected from SEQ ID NOs: 43-45; (iv) one or more CDRs selected from SEQ ID NOs: 49-51; or (v) one or more CDRs selected from SEQ ID NOs: 61-63. In one embodiment, the anti-H5 monoclonal antibody light chain or a fragment thereof comprises three CDRs having the amino acid sequences set forth in SEQ ID NOs: 31-33, respectively. In another embodiment, the anti-H5 monoclonal antibody light chain or a fragment thereof comprises three CDRs having the amino acid sequences set forth in SEQ ID NOs: 37-39, respectively. In another embodiment, the anti-H5 monoclonal antibody light chain or a fragment thereof comprises three CDRs having the amino acid sequences set forth in SEQ ID NOs: 43-45. In another embodiment, the anti-H5 monoclonal antibody light chain or a fragment thereof comprises three CDRs having the amino acid sequences set forth in SEQ ID NOs: 49-51. In another embodiment, the anti-H5 monoclonal antibody light chain or a fragment thereof comprises three CDRs having the amino acid sequences set forth in SEQ ID NOs: 61-63.

In another aspect, the CDRs contained in the anti-H5 monoclonal antibody light chains or fragments thereof of the present invention may include one or more amino acid substitution, addition and/or deletion from the amino acid sequences set forth in SEQ ID NOs: 31-33, 37-39, 43-45, 49-51, or 61-63. Preferably, the amino acid substitution, addition and/or deletion occur at no more than three amino acid positions. More preferably, the amino acid substitution, addition and/or deletion occur at no more than two amino acid positions. Most preferably, the amino acid substitution, addition and/or deletion occur at no more than one amino acid position.

The variants generated by amino acid substitution, addition and/or deletion in the variable regions of the above described antibodies or the above described CDRs maintain the ability of specifically binding to subtype H5 of avian influenza virus. The present inventions also include antigen-binding fragments of such variants.

Monoclonal antibody variants of the invention may be made by conventional genetic engineering methods. Nucleic acid mutations may be introduced into the DNA molecules using methods known to a person with ordinary skill in the art. Alternately, the nucleic acid molecules encoding the heavy and light chain variants may be made by chemical synthesis.

Chimeric Antibodies, Humanized Antibodies and Fusion Proteins

In another aspect, the present invention also provides chimeric antibodies that comprise, in whole or in part, the heavy and/or light chain variable regions of murine monoclonal antibodies 8H5, 3C8, 10F7, 4D1, 3G4 or 2F2 or a variant thereof, combined with the constant regions of a human monoclonal antibody. Additionally, the present invention includes humanized antibodies that comprise one or more of the CDRs of murine monoclonal antibodies 8H5, 3C8, 10F7, 4D1, 3G4 or 2F2 or a variant thereof, grafted into a human antibody framework.

In another aspect, the present invention provides a fusion protein comprising, in whole or in part, the monoclonal antibody of the invention, conjugated with another molecule or molecules.

The chimeric antibodies, humanized antibodies and fusion proteins disclosed herein may be produced by conventional genetic engineering methods. For example, DNA encoding the monoclonal antibodies may be modified by substituting the coding sequence for human heavy and light chain constant domains in place of the homologous murine sequences (Morrison, et al., Proc. Nat. Acad. Sci. 81: 6851 (1984)), or by covalently joining to the immunoglobulin coding sequence all or part of the coding sequence for a non-immunoglobulin polypeptide to produce the chimeric or humanized antibodies as well as the fusion proteins.

Neutralizing Antibodies

In another aspect, the present invention provides anti-H5 antibodies that are capable of neutralizing the viral activity of subtype H5 avian influenza virus. In one embodiment, such neutralizing antibodies are capable of neutralizing at least 60%, or at least 70%, preferably at least 75%, more preferably at least 80%, more preferably at least 85%, more preferably at least 90%, even more preferably at least 95%, most preferably at least 99% of the viral activity of subtype H5 avian influenza virus.

The ability of an antibody to neutralize the viral activity of subtype H5 avian influenza virus is assayed using conventional methods known to a person with ordinary skill in the art. Example 1 describes the procedures of a neutralizing assay used by the inventors to determine the neutralizing activity of certain anti-H5 monoclonal antibodies of the invention.

Short Peptides

In another aspect, the present invention provides short peptides that simulate the antigen sites binding of the mAbs provided herein.

Nine short peptides which have 7 amino acids have been identified based on their ability to bind to 8H5 mAb or 3C8 mAb. Five of these peptides having the sequences set forth in SEQ ID NOs: 64-68 show binding to 8H5 mAb, and four of the peptides having the sequences set forth in SEQ ID NOs: 70-73 show binding to 3C8 mAb (Table 14).

The 7-aa peptides 8H5A (SEQ ID NO: 64) and 8H5E (SEQ ID NO: 68) demonstrate the specific reactions. The reaction between the peptide 8H5A and the monoclonal antibody 8H5 is particularly good, but the specific reactions between 8H5A and the other three monoclonal antibodies were weak. The specific reaction between 8H5E and monoclonal antibody 8H5 was relatively poor.

Furthermore, 12 short peptides (Table 16, SEQ ID NOS: 74-97 for amino acid sequence and base sequence) having 12 amino acids each have been identified based on their binding specificity for 8H5 mAb.

The 12aa peptides with peptide Section Nos. 123 or 125 were used to make the fusion proteins 239-123 and 239-125, which exhibit specificity for 8C11 and 8H5, respectively. The fusion proteins of the 12aa peptides 123 and 125 with HBVcAg also showed binding specificity for 8H5, but not for other mAbs. The fusion proteins HBc-122, HBc-124, HBc-128 and HBc-129 reacted only with 8H5, and did not react with any other mAb. It has been demonstrated that virus-like particles assembled from fusion proteins HBc-123, HBc-124, HBc-125, HBc-128 or HBc-129 each simulate some part of the antigen site binding to 8H5 mAb.

Detection Methods

The present invention further provides a method for detecting the presence of the antigen and/or antibody of subtype H5 of avian influenza virus in a sample using a monoclonal antibody of the invention.

In one aspect, the present invention provides a method for detecting the presence of subtype H5 of avian influenza virus in a sample comprising the steps of: (i) contacting said sample with an monoclonal antibody or a fragment thereof of the invention to form a complex of said antibody or fragment with said virus, and (ii) detecting said complex to determine the presence of said virus in said sample.

In another aspect, the present invention provides a method for detecting the presence of subtype H5 of avian influenza virus in a sample comprising the steps of: (i) attaching a first antibody to a solid substrate; (ii) adding a sample suspected of having subtype H5 of avian influenza virus to said substrate; (iii) adding a second antibody that is linked to a labeling agent to said substrate; (iv) detecting the presence of the labeling agent to measure the presence of subtype H5 of avian influenza virus.

In another aspect, the present invention provides a method for detecting the presence of subtype H5 of avian influenza virus in a sample comprising the steps of: (i) attaching an antibody to a solid substrate; (ii) adding a sample suspected of having subtype H5 of avian influenza virus pre-mixed with labeled H5 hemagglutinin to said substrate; (iii) detecting the presence of the labeled H5 hemagglutinin.

The detection methods may use enzyme-linked immunosorbent assay (ELISA), enzyme immunoassay, chemiluminescence immunoassay, radioimmunoassay, fluorescence immunoassay, immunochromatography, competition assay and like techniques. The detection methods can be used to detect the target antigens or antibodies via competition or sandwich methods.

The competition method is based on the quantitative competitive binding of an antigen in a sample and a known amount of a labeled antigen to the monoclonal antibody of the present invention. To carry out an immunological assay based on the competition method, a sample containing an unknown amount of the target antigen is added to a solid substrate to which the monoclonal antibody of the present invention is bound physically or chemically by known means, and the reaction is allowed to proceed. Simultaneously, a predetermined amount of the target antigen pre-labeled with a labeling agent is added and the reaction is allowed to proceed. After incubation, the solid substrate is washed and the activity of the labeling agent bound to the solid substrate is measured.

In the sandwich method, the target antigen in a sample is sandwiched between the immobilized monoclonal antibody of the invention and the monoclonal antibody of the invention labeled with a labeling agent, then a substrate for the labeling agent such as an enzyme is added, substrate color changes are detected, and thereby detecting the presence of the antigen. To carry out an immunological assay based on the sandwich method, a sample containing an unknown amount of the target antigen, for instance, is added to a solid substrate to which the monoclonal antibody of the present invention is bound physically or chemically by known means, and the reaction is allowed to proceed. Thereafter, the monoclonal antibody of the invention labeled with a labeling agent is added and the reaction is allowed to proceed. After incubation, the solid substrate is washed and the activity of the labeling agent bound to the solid substrate is measured. The labeling agent may be radioisotopes such as ¹²⁵I, enzymes, enzyme substrates, luminescent substances such as isoluminol and acridine esters, fluorescent substances such as fluorescein and rhodamine, biotin, and colored substances such as colored latex particles and colloidal gold. Labeling enzymes may be peroxidase (e.g. Horse Radish Peroxidase (HRP)), alkaline phosphatase, β-galactosidase, and glucose oxidase. Suitable substrates for the reactions may be selected from ABTS, luminol-H₂O₂, o-phenylenediamine-H₂O₂ (against peroxidase), p-nitrophenyl phosphate, methylumbelliferyl phosphate, 3-(2′-spiroadamantan)-4-methoxy-4-(3″-phosphoryloxy)phenyl-1,2-dioxetane (against alkaline phosphatase), p-nitrophenyl-β-D-galactose, and methylumbelliferyl-β-D-galactose (against β-galactosidase). Additional labels include quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as alkaline phosphatase, horseradish peroxidase, I²-galactosidase, alkaline phosphatase, β-galactosidase, or acetylcholinesterase and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, a luminescent material such as luminol; light scattering or plasmon resonant materials such as gold or silver particles or quantum dots; or radioactive material include ¹⁴C, ¹²³I, ¹²⁴I, ¹³¹I, Tc99m, ³⁵S or ³H; or spherical shells, and probes labeled with any other signal generating label known to those of skill in the art. For example, detectable molecules include but are not limited to fluorophores as well as others known in the art as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland. In some embodiments, labels comprise semiconductor nanocrystals such as quantum dots (i.e., Qdots), described in U.S. Pat. No. 6,207,392. Qdots are commercially available from Quantum Dot Corporation. The semiconductor nanocrystals useful in the practice of the invention include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV semiconductors such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same.

In some embodiments, a fluorescent energy acceptor is linked as a label to a detection probe In one embodiment the fluorescent energy acceptor may be formed as a result of a compound that reacts with singlet oxygen to form a fluorescent compound or a compound that can react with an auxiliary compound that is thereupon converted to a fluorescent compound. Such auxiliary compounds can be comprised in buffers contained in a device of the invention. In other embodiments, the fluorescent energy acceptor may be incorporated as part of a compound that also includes the chemiluminescer. For example, the fluorescent energy acceptor may include a metal chelate of a rare earth metal such as, e.g., europium, samarium, tellurium and the like. These materials are particularly attractive because of their sharp band of luminescence. Furthermore, lanthanide labels, such as europium (III) provide for effective and prolonged signal emission and are resistant to photo bleaching, thereby allowing Test Devices containing processed/reacted sample to be set aside if necessary for a prolong period of time. Long-lifetime fluorescent europium(III) chelate nanoparticles have been shown to be applicable as labels in various heterogeneous and homogeneous immunoassays. See, e.g., Huhtinen et al, Clin. Chem., October 2004, 50(10): 1935-6. Assay performance can be improved when these intrinsically labeled nanoparticles are used in combination with time-resolved fluorescence detection. In heterogeneous assays, the dynamic range of assays at low concentrations can be extended. Furthermore, the kinetic characteristics of assays can be improved by use of detection antibody-coated high-specific-activity nanoparticle labels instead of conventionally labeled detection antibodies. In homogeneous assays, europium(III) nanoparticles have been shown to be efficient donors in fluorescence resonance energy transfer, enabling simple and rapid highthroughput screening. In some embodiments, a label (e.g., fluorescent label) disclosed herein, is comprised as a nanoparticle label conjugated with biomolecules. In other words, a nanoparticle can be utilized with a detection or capture probe. For example, a europium(III)-labeled nanoparticle linked to monoclonal antibodies or streptavidin (SA) to detect a particular analyte in a sample can be utilized in practice of the present invention (e.g., nanoparticle-based immunoassay). The nanoparticles serve as a substrate to which are attached the specific binding agents to the analyte and either the detection (i.e., label) or capture moiety. Examples of labels can also be found in U.S. Pat. Nos. 4,695,554, 4,863,875, 4,373,932, and 4,366,241. Colloidal metals and dye particles are disclosed in U.S. Pat. Nos. 4,313,734 and 4,373,932. The preparation and use of non-metallic colloidals are disclosed in U.S. Pat. No. 4,954,452. Organic polymer latex particles for use as labels are disclosed in U.S. Pat. No. 4,252,459.

The labeling agents may be bound to the antigen or antibody by maleimide method (J. Biochem. (1976), 79, 233), activated biotin method (J. Am. Chem. Soc. (1978), 100, 3585), hydrophobic bond method, activated ester method or isocyanate method (“Enzyme immunoassay techniques”, published in 1987 by Igaku Shoin).

When the above labeling agent is radioisotopes, the measurement is carried out using a well counter or a liquid scintillation counter. When the labeling agent is an enzyme, the substrate is added and the enzyme activity is measured by colorimetry or fluorometry. When the labeling agent is a fluorescent substance, luminescent substance or colored substance, the measurement can be made respectively by a method known in the art.

In this invention, the samples used for detecting subtype H5 of avian influenza virus include but are not limited to the wastes from the animals or patients, secretions from the mouth and nasal cavities, intact virus or lytic virus liquid in the chick embryo culture, etc.

Detection Devices and Kits

This invention further relates to a kit for diagnosis of the infection by subtype H5 of avian influenza virus, especially to a kit for detecting the antigen or antibody of subtype H5 of avian influenza virus in the sample. The diagnosis kit of the invention comprises at least one monoclonal antibody species of the invention. The monoclonal antibody of the invention, which is to be used in the diagnosis kit of the invention, is not particularly restricted but may be any of those recognizing the H5 hemagglutinin antigen, and may be any antigen-binding fragments of the monoclonal antibodies of the invention such as F(ab′)2, Fab′, Fab and the like.

In one aspect, this invention relates to two kinds of kits for detecting subtype H5 of avian influenza virus which contain at least one of the monoclonal antibodies of the invention or their active fragments or variants. Preferably, the kits of the present invention contain the detecting reagent suitable for detecting the antigen-antibody reactions.

In another aspect, this invention relates to a kit for detecting anti-H5 avian influenza virus, which contains at least one of the monoclonal antibodies of the invention or their active fragments or variants. Preferably, the kit mentioned in this invention contains the detection reagent suitable for detecting the antigen-antibody reactions.

A solid substrate, or a solid phase substrate, to be used in the detection methods according to the present invention, includes without limitation microplates, magnetic particles, filter papers for immunochromatography, polymers such as polystyrene, glass beads, glass filters and other insoluble carriers. In one embodiment, a solid phase substrate comprising a plurality of compartments or areas, wherein at least one compartment is coated with antibodies of the present invention. In a preferred embodiment, at least one compartment (or a first compartment) is coated with an antibody of the present invention and at least one remaining compartment (or a second compartment) is coated with an antibody that specifically binds to an avian fluenza virus subtype other than H5 (e.g., H1, H2, H3, H4, H6, H7, H9, H10, H11, H12, H113, H13, H14, H15 or H16), preferably subtype H1, H3, H7, or H9.

The diagnosis kit of the invention may further comprise other constituents. The other constituents include without limitation enzymes for labeling, substrates therefor, radioisotopes, luminescent substances, fluorescent substances, colored substances, buffer solutions, and plates, and those mentioned hereinabove can be used as these.

In the diagnosis kit of the invention, the monoclonal antibody of the invention may be immobilized on a solid substrate in advance. In a preferred embodiment, the monoclonal antibody is immobilized on the solid substrate in an orientation that enhances the binding efficiency of the antibody to the antigen TaeWoon Cha et al (Proteomics 5, 416-419 (2005)) demonstrated that controlling the orientation of immobilized protein molecules and designing an ideal local chemical environment on the solid substrate surface are important for preserving and enhancing the reaction activity and efficiency of the immobilized proteins. Various methods for attaching antibodies to a solid substrate in a desired orientation have been reported. Shawn Weng et al (Proteomics 2, 48-57 (2002)) reported a method of orienting proteins in a uniform manner on a surface through nucleic acids linked to the proteins. Soellner, M. et al (J. AM. CHEM. SOC., 125, 11790-11791 (2003)) disclosed a method pursuant to which proteins including antibodies and antigens were immobilized to a surface in a uniform manner through Staudinger ligation in which an azide and phosphinothioester react to form an amide. Hairong Zhang et al (Anal. Chem., 78, 609-616 (2006)) disclosed a method of orienting antibodies on gold-coated magnetic particles through reaction of the free thiols of the Fab′ fragments of the antibodies to the surface of the particles, pursuant to which all the antigen binding sites of the antibodies were oriented in a favorable direction. Hai Xu et al. (J. Phys. Chem. B, 110, 1907-1914 (2006)) reported methods of adsorbing antibodies to the hydrophilic silicon oxide/water surface. Seung-yong Seong et al. (Proteomics, 3, 2176-2189 (2003)) provided an overview of methods for oriented immobilization of proteins to a surface and protein molecules used in such methods. All these references are incorporated herein in their entirety.

In the diagnosis kit of the invention, the monoclonal antibody of the invention or the antigen may be labeled with the above-mentioned labeling agent in advance.

The present invention further provides an automated device that is capable of detecting avian influenza virus in a sample through automated processes.

Various devices for detecting the presence of an analyte in a sample of biological fluid through the use of immunochemistry have been described in the art. Devices may utilize the so-called “sandwich” assay, for example, a target analyte such as an antigen is “sandwiched” between a labeled antibody and an antibody immobilized onto a solid support. The assay is read by observing the presence and/or amount of bound antigen-labeled antibody complex. Devices may also incorporate a competition immunoassay, wherein an antibody bound to a solid surface is contacted with a sample containing an unknown quantity of antigen analyte and with labeled antigen of the same type. The amount of labeled antigen bound on the solid surface is then determined to provide an indirect measure of the amount of antigen analyte in the sample. Various assays utilize devices adapted to assay a plurality of different analytes, for example, by incorporating different antibodies or antigen in designated or addressable regions of the test substrate (e.g., bibulous or non-bibulous membranes). Because these and other methods discussed below can detect both antibodies and antigens, they are generally referred to as immunochemical ligand-receptor assays or simply immunoassays.

Solid phase immunoassay devices, whether sandwich or competition type, provide sensitive detection of an analyte in a biological fluid sample such as blood or urine. Solid phase immunoassay devices incorporate a solid support to which one member of a ligand-receptor pair, usually an antibody, antigen, or hapten, is bound. Common early forms of solid supports were plates, tubes, or beads of polystyrene which were well known from the fields of radioimmunoassay and enzyme immunoassay. More recently, a number of porous materials such as nylon, nitrocellulose, cellulose acetate, glass fibers, and other porous polymers have been employed as solid supports. A number of self-contained immunoassay kits using porous materials as solid phase carriers of immunochemical components such as antigens, haptens, or antibodies have been described. These kits are usually dipstick, flow-through, or migratory in design. Any of the conventional, well-known devices for performing immunoassays or specific binding assays may be utilized in the invention to detect influenza.

In certain aspects of the invention, it is included with devices for diagnosis of infection caused by various influenza virus types or subtypes. In some embodiments, a sample that may contain one or more influenza virus or anti-influenza virus antibodies is administered to a device to determine if the sample is from a subject infected with one or more influenza virus type or subtype. A device comprising a solid support can comprise anti-influenza virus antibodies or influenza virus antigens disposed thereon, thus providing a means to test a sample suspected of containing an influenza virus, influenza virus protein or an anti-influenza virus antibody. In various embodiments, antibodies utilized in devices of the invention include but are not limited to: a polyclonal, a monoclonal antibody (MAb), or conservative or functional variants thereof, a chimeric antibody, a reshaped antibody, a humanized antibody, a bioactive fragment thereof, or any combination of one or more such antibodies; any of such functional antibodies or fragments thereof may be referred to herein collectively as antibody or the plural antibodies. Antibodies of the invention can be adapted to any devices to allow detection of influenza virus. For example, H5 avian influenza virus can be detected by targeting of an H5 protein or an anti-subtype H5 antibody in a sample. In one embodiment, H5 is from Avian Influenza Virus (AIV).

Many devices are commercially available which can be easily adapted to incorporate antibodies or antigens disclosed herein. Devices can incorporate solid substrate to be used in the detection methods, including without limitation microplates, magnetic particles, filter papers for immunochromatography, polymers such as polystyrene, glass beads, glass filters and other insoluble carriers. The substrate generally will be in shapes including but not limited to a strip, sheet, chip, sphere, bead or well, such as a well in micro titer plate, or any other shapes that are suitable. Furthermore, the substrate to which a binding partner (i.e., antigen or antibody) is bound may be in any of a variety of forms, e.g., a microtiter dish, a test tube, a dipstick, a microcentrifuge tube, a bead, a spinnable disk, and the like. Suitable materials include glass, plastic (e.g., polyethylene, PVC, polypropylene, polystyrene, and the like), protein, paper, carbohydrate, and other solid supports. Other materials that may be employed include ceramics, metals, metalloids, semiconductive materials, cements and the like. In some embodiments, microtiter plates utilized in immunoassays (e.g., ELISA) can comprise 96 well, 384 well plates or 1536 well formats, or higher number wells, such as in other commercially available plates.

Some exemplary devices include dipstick, lateral flow, cartridge, multiplexed, microtiter plate, microfluidic, plate or arrays or high throughput platforms, such as those disclosed in U.S. Pat. Nos. 6,448,001, 4,943,522, 6,485,982, 6,656,744, 6,811,971, 5,073,484, 5,716,778, 5,798,273, 6,565,808, 5,078,968, 5,415,994, 6,235,539, 6,267,722, 6,297,060, 7,098,040, 6,375,896, 7,083,912, 5,225,322, 6,780,582, 5,763,262, 6,306,642, 7,109,042, 5,952,173, and 5,914,241. Exemplary microfluidic devices include those disclosed in U.S. Pat. No. 5,707,799 and WO2004/029221.

Dipstick

In the more common forms of dipstick assays, as typified by home pregnancy and ovulation detection kits, immunochemical components such as antibodies are bound to a solid phase. The assay device is “dipped” for incubation into a sample suspected of containing unknown antigen analyte. Alternatively a small amount of sample can be placed onto a sample receiving zone. A labeled antibody is then added and the label is detected as an indication of the presence of the analyte of interest. In some cases the label is an enzyme so an enzyme-labeled antibody is then added, either simultaneously or after an incubation period. The device next is washed and then inserted into a second solution containing a substrate for the enzyme. The enzyme-label, if present, interacts with the substrate, causing the formation of colored products which either deposit as a precipitate onto the solid phase or produce a visible color change in the substrate solution. Baxter et al., EP-A 0 125 118, disclose such a sandwich type dipstick immunoassay. Kali et al., EP-A 0 282 192, disclose a dipstick device for use in competition type assays. The materials for the dipstick, formats and labels are well-known and can be adapted for an influenza assay. Exemplary dipstick devices include those described in U.S. Pat. Nos. 4,235,601, 5,559,041, 5,712,172, and 6,790,611. In some embodiments antibodies of the invention can be disposed onto a dipstick device. For example, anti-subtype H5 AIV antibody is detected in a sample through the use of a solid phase support dipstick onto which is attached at one or more matrix squares. One matrix square has a non-specific control antibody attached and one to which has been attached an antibody or functional fragment thereof. These matrix squares are the sites of protein-binding and/or antigen-binding in and are usually made of nitrocellulose; however, any suitable medium known in the art can be utilized, such as certain nylons and polyvinylidenes. In some embodiments a multitude of matrices are attached to the solid support, each matrix containing an antigen or antibody for a plurality of influenza virus subtypes.

Flow-Through

Flow-through type immunoassay devices were designed to obviate the need for extensive incubation and cumbersome washing steps associated with dipstick assays. Valkirs et al., U.S. Pat. No. 4,632,901, disclose a device comprising antibody (specific to a target antigen analyte) bound to a porous membrane or filter to which is added a liquid sample. As the liquid flows through the membrane, target analyte binds to the antibody. The addition of sample may be followed by addition of labeled antibody. The visual detection of labeled antibody provides an indication of the presence of target antigen analyte in the sample. Korom et al., EP-A 0 299 359, discloses a variation in the flow-through device in which the labeled antibody is incorporated into a membrane which acts as a reagent delivery system. Such devices may comprise layers which serve as filters for components in the sample and include the reagents utilized in the assay. As the sample flows from one layer to another, it contacts and reacts with the specific binding reagents and in some instances, the components of the labeling system to provide an indication of the presence of the analyte.

Immunofiltration Devices

Immunofiltration devices are commercially available (e.g., Pierce, Rockford, Ill.) and can be easily adapted to incorporate antibodies of the invention. In an enzyme-linked immunoflow assay (ELIFA) method uses a nitrocellulose membrane sandwiched between a 96-well sample application plate and a vacuum chamber. Reactants are added to the sample application plate and the vacuum pulls reactants through the membrane. Cannulas transfer nonbound products to the collection chamber. For detection, a microplate is placed in the collection chamber before adding the enzyme substrate. The vacuum allows transference of the colored product into microplate wells for analysis in an automated microplate reader. The ELIFA system is composed of precision cut plexiglass with tight sealing gaskets that provide constant flow rates from well to well. The cannulas precisely transfer colored product to microplate wells for analysis. Basically, a capture antibody of the invention is spotted on the substrate (e.g., microtiter plate, membrane or chip). A biological sample suspected of containing influenza virus or influenza virus antigens are applied and incubated to allow the capture antibodies to bind. Subsequently, a detection antibody added. An example of high-throughput immunofiltration device is disclosed in U.S. Patent Application 2003/0108949. Such devices may comprise layers which serve as filters and/or include the reagents utilized in the assay. As the sample reacts with the specific binding reagents and in some cases components of the labeling system to provide an indication of the presence of an analyte.

Lateral Flow Devices

In lateral flow type assays, a membrane is impregnated with the some or all of the reagents needed to perform the assay. An analyte detection zone is provided in which labeled analyte is detected. See, for example, Tom et al., U.S. Pat. No. 4,366,241, and Zuk, EP-A 0 143 574. Many variations are known for lateral flow assay devices. The device may contain some of the reagents for the specific binding assay (the sample may be reacted with some reagents prior to application to the lateral flow strip or additional reagents may be sequentially applied to the strip) or the strip may contain all of the necessary reagents for the specific binding assay. Lateral flow devices most frequently incorporate within them reagents which have been attached to colored labels, thereby permitting visible detection of the assay results without addition of further substances. See, for example, Bernstein, U.S. Pat. No. 4,770,853, May et al., WO 88/08534, and Ching et al., EP-A 0 299 428. The devices are generally constructed to include a location for the application of the sample, a reagent zone and a detection zone. The device is typically made from a bibulous material which permits the sample to flow through the membrane from the sample application zone through the reagent or reaction zone to the detection zone(s). While some of the reactions may occur before application of the sample to the strip, in some embodiments, the reaction zone(s) include the reagents for the immunoassay. One specific binding reagent, for example an antibody, may be diffusively bound to the strip in the sample application zone or a reaction zone so that it can bind the antigen in the sample and flow with the sample along the strip. The antigen-antibody complex may be captured in the detection zone directly with another specific binding partner to the antigen or antibody or it may be captured indirectly using additional specific binding partners, such as avidin or streptavidin and biotin. Similarly, the label may be directly or indirectly attached to the antigen or antibody. Exemplary lateral flow devices include those described in U.S. Pat. Nos. 4,818,677, 4,943,522, 5,096,837 (RE 35,306), U.S. Pat. Nos. 5,096,837, 5,118,428, 5,118,630, 5,221,616, 5,223,220, 5,225,328, 5,415,994, 5,434,057, 5,521,102, 5,536,646, 5,541,069, 5,686,315, 5,763,262, 5,766,961, 5,770,460, 5,773,234, 5,786,220, 5,804,452, 5,814,455, 5,939,331, 6,306,642. Other lateral flow devices that may be modified for use in distinguishable detection of multiple analytes in a fluid sample include U.S. Pat. Nos. 4,703,017, 6,187,598, 6,352,862, 6,485,982, 6,534,320 and 6,767,714.

It is also conventional to assay multiple analytes from a sample using a single test strip by establishing separate detection zones for each analyte. Distinguishing between different analytes can be accomplished by using different labels or by measuring the same label in the different detection zones. Assaying for multiple analytes can be accomplished with any of the conventional devices.

Immunoassays utilize mechanisms of the immune systems, wherein antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a test sample.

Such a lateral flow device usually comprises a porous membrane optionally supported by a rigid material. In general, the porous membrane may be made from any of a variety of materials through which a fluid is capable of passing. For example, the materials used to form the porous membrane may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO4, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and the like. In one particular embodiment, the porous membrane is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

Such a device may also contain a strip with an absorbent pad disposed upstream or downstream of the test/detection or control zones. As is well known in the art, the absorbent pad may assist in promoting capillary action and fluid flow through the membrane. In some embodiments, absorbent pads may contain mobilizable immunoassay reagents (e.g., antibodies). Of course, it is understood the mobilizable or immbolized immunoassay reagents can also be disposed anywhere upstream of the detection/test or control zones, as well as in separate components of a detection system.

In various embodiments, some suitable materials that may be used to form the sample pad include, but are not limited to, nitrocellulose, cellulose, porous polyethylene pads, and glass fiber filter paper. If desired, the sample pad may also contain one or more assay pretreatment reagents, either covalently or non-covalently attached thereto. The test sample travels from the sample pad to a conjugate pad that is placed in communication with one end of the sampling pad. The conjugate pad is formed from a material through which a fluid is capable of passing. For example, in one embodiment, the conjugate pad is formed from glass fibers. It should be understood that other conjugate pads may also be used in the present invention. Alternatively, in some embodiments conjugates or other immunoreagents may be included in a component that is mixed with a sample prior to application to a test strip.

To facilitate detection of the presence or absence of an analyte within the test sample, various detection probes may be applied to the conjugate pad. While contained on the conjugate pad, these detection probes remain available for binding with the analyte as it passes from the sampling pad through the conjugate pad (or optionally in the fluid). Upon binding with the analyte, the detection probes may later serve to identify the presence or absence of the analyte. The detection probes may be used for both detection and calibration of the assay. In alternative embodiments, however, separate calibration probes may be applied to the conjugate pad for use in conjunction with the detection probes to facilitate simultaneous calibration and detection, thereby eliminating inaccuracies often created by conventional assay calibration systems. It should be understood, however, that the detection probes and/or the calibration probes may be applied together or separately at any location of the assay, and need not be applied to the conjugate pad. Further, it should also be understood that the detection probes and/or the calibration probes may be applied to the same or different conjugate pads. Alternatively, the detection probes and/or calibration probes may be located in a separate area of the diagnostic test unit, for example in self-contained test devices, such as within the fluid, a flow channel, or a swab.

In some instances, it may be desired to modify the detection probes in some manner so that they are more readily able to bind to the analyte. In such instances, the detection probes may be modified with certain specific binding members that are adhered thereto to form conjugated probes. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are disclosed herein. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

In one embodiment, for instance, the fluid containing the test sample travels to the conjugate pad, where the analyte mixes with detection probes modified with a specific binding member to form analyte complexes. Because the conjugate pad is in fluid communication with the porous membrane, the complexes may migrate from the conjugate pad to a detection zone present on the porous membrane. Alternatively, multiple detection zones can be utilized by incorporating antibodies specific for different antigens (e.g., different influenza virus or viral antigens from different influenza virus). The detection zone(s) may contain an immobilized reagent that is generally capable of forming a chemical or physical bond with the analyte and/or complexes thereof (e.g., complexes of the analyte with the detection probes). In some embodiments, the reagent may be a biological reagent, such as antibodies disclosed herein. Other biological reagents are well known in the art and may include, but are not limited to, antigens, haptens, antibodies, protein A or G, avidin, streptavidin, and complexes thereof. In some cases, it is desired that these biological reagents are capable of binding to the analyte and/or the complexes of the analyte with the detection probes.

These reagents serve as stationary binding sites for the detection probe/analyte complexes. In some instances, the analytes, such as antibodies, antigens, etc., have two binding sites. Upon reaching the detection zone(s), one of these binding sites is occupied by the specific binding member of the complexed probes. However, the free binding site of the analyte may bind to the immobilized reagent. Upon being bound to the immobilized reagent, the complexed probes form a new ternary sandwich complex.

The detection or test zone(s) may generally provide any number of distinct detection regions so that a user may better determine the presence of a particular analyte within a test sample. Each region may contain the same reagents, or may contain different reagents for capturing multiple analytes. For example, the detection zone(s) may include two or more distinct detection regions (e.g., lines, dots, etc.). The detection regions may be disposed in the form of lines in a direction that is substantially perpendicular to the flow of the test sample through the assay. Likewise, in some embodiments, the detection regions may be disposed in the form of lines in a direction that is substantially parallel to the flow of the test sample through the assay device.

In some cases, the membrane may also define a control zone (not shown) that gives a signal to the user that the assay is performing properly. For instance, the control zone (not shown) may contain an immobilized reagent that is generally capable of forming a chemical and/or physical bond with probes or with the reagent immobilized on the probes. Some examples of such reagents include, but are not limited to, antigens, haptens, antibodies, protein A or G, avidin, streptavidin, secondary antibodies, and complexes thereof. In addition, it may also be desired to utilize various non-biological materials for the control zone reagent. For instance, in some embodiments, the control zone reagent may also include a polyelectrolyte, such as described above, that may bind to uncaptured probes. Because the reagent at the control zone is only specific for probes, a signal forms regardless of whether the analyte is present. The control zone may be positioned at any location along the membrane, but is preferably positioned upstream from the detection zone.

Various formats may be used to test for the presence or absence of an analyte using the assay. For instance, in the embodiment described above, a “sandwich” format is utilized. Other examples of such sandwich-type assays are described by U.S. Pat. Nos. 4,168,146 to Grubb et al. and 4,366,241 to Tom et al., which are incorporated herein in their entirety by reference thereto for all purposes. In addition, other formats, such as “competitive” formats, may also be utilized. In a competitive assay, the labeled probe is generally conjugated with a molecule that is identical to, or an analogue of, the analyte. Thus, the labeled probe competes with the analyte of interest for the available reagent. Competitive assays are typically used for detection of analytes such as haptens, each hapten being monovalent and capable of binding only one antibody molecule. Examples of competitive immunoassay devices are described in U.S. Pat. Nos. 4,235,601 to Deutsch et al., 4,442,204 to Liotta, and 5,208,535 to Buechler et al., which are incorporated herein in their entirety by reference thereto for all purposes. Various other device configurations and/or assay formats are also described in U.S. Pat. No. 5,395,754 to Lambofte et al.; U.S. Pat. Nos. 5,670,381 to Jou et al.; and 6,194,220 to Malick et al., which are incorporated herein in their entirety by reference thereto for all purposes.

Microfluidic Devices

In some aspects of the invention, antibodies disclosed herein can be incorporated into a microfluidic device. The device is a microfluidic flow system capable of binding one or more analytes. The bound analytes may be directly analyzed on the device or be removed from the device, e.g., for further analysis or processing. Alternatively, analytes not bound to the device may be collected, e.g., for further processing or analysis.

An exemplary device is a flow apparatus having a flat-plate channel through which a sample flows; such a device is described in U.S. Pat. No. 5,837,115. Samples can travel through such device drive by gravity, capillary or by an active force, such as by an infusion pump to perfuse a sample, e.g., blood, through the microfluidic device. Other pumping methods, as known in the art, may be employed. Microfluidic devices may optionally rely on an array of structures in the device for analyte capture. The structures can be made by a variety of processes including, but not limited to lasering, embossing, Lithographie Galvanoformung Abformung (LIGA), electroplating, electroforming, photolithography, reactive ion etching, ion beam milling, compression molding, casting, reaction injection molding, injection molding, and micromachining the material. As will be understood, the methods utilized to manufacture the devices of the present invention are not critical as long as the method results in large quantities of uniform structures and devices. Furthermore, the method must result in a large surface area of the structure and arranged in close proximity to each other to produce narrow channels. The narrow channels allow analyte diffusion in the fluid to occur to enhance the efficiency of capturing analyte and/or labelled reagent at the capture site.

The mass produced structures are preferably made of any number of polymeric materials. Included among these are, but not intended to be limited to, polyolefins such as polypropylene and polyethylene, polyesters such as polyethylene terephthalate, styrene containing polymers such as polystyrene, styreneacrylonitrile, and acrylonitrilebutadienestyrene, polycarbonate, acrylic polymers such as polymethylmethacrylate and poly acrylonitrile, chlorine containing polymers such as polyvinylchloride and polyvinylidenechloride, acetal homopolymers and copolymers, cellulosics and their esters, cellulose nitrate, fluorine containing polymers such as polyvinylidenefluoride, polytetrafluoroethylene, polyamides, polyimides, polyetheretherketone, sulfur containing polymers such as polyphenylenesulfide and polyethersulfone, polyurethanes, silicon containing polymers such as polydimethylsiloxane. In addition, the structures can be made from copolymers, blends and/or laminates of the above materials, metal foils such as aluminum foil, metallized films and metals deposited on the above materials, as well as glass and ceramic materials. In one such method, a laser, such as an excimer laser can be used to illuminate a photomask so that the light that passes through the photomask ablates an underlying material forming channels in the material substrate. Sercel, J., et al., SPIE Proceedings, Vol. 998, (September, 1988).

Generally, microfluidic devices can comprise an inlet port to which the test sample is initially presented. Generally, the channels are capillaries and provide transport of the test sample from the inlet port through the device, an array of structures which provide a capture site, and a vent, such as an exit port, which vents gases in the device. In addition, chambers and additional capillaries may be added to customize a device. Generally, test sample movement through the device relies on capillary forces. In addition, one or more capillaries can be used to bring the test sample from the inlet port to the channels. Additionally, one or more capillaries can be used to exit the structures area of the device. However, differential pressure may be used to drive fluid flow in the devices in lieu of, or in addition to capillary forces.

Channels are created between adjacent structures through which fluid can flow. Both the channel and structure designs are important to optimize contact between the structure surfaces and fluid molecules. Typically, the depth of the channels range from about 1 micrometer (μm) to about 1 millimeter (μm). The average width of the channels typically range from about 0.02 μm to 20 μp. The channels may include structures of various shapes, including diamonds, hexagons, circles, or squares with height ranges typically from about 1 .mu.m to 1 mm and the average width typically ranges from 1 μm to 1 mm.

Immobilized reagent can be covalently or non-covalently attached onto the surface of the structures as well as within the capillaries and/or chambers. The reagent can be applied as a time-released reagent, spatially separated reagent, or coated and dried onto the surface. Such techniques of placing immobilized reagent on the surfaces are well known to those skilled in the art. In one embodiment, the immobilized reagents are antibodies disclosed herein which target influenza virus antigens (e.g., H5 AIV).

The methods for utilizing devices of the present invention involve specific binding members. The methods of detection may involve the binding of a colored label such as a fluorescent dye or a colored particle. Alternatively, detection may involve binding of an enzyme which can produce a colored product.

One or more alternate flow paths can be used in the devices of the present invention. The capillary transporting the test sample from the inlet port branches in different pathways, the main pathway to the structures and the alternate pathways. The alternate pathways can-allow for multiple capture sites and allow simultaneous determinations of the presence or amount of multiple analytes in a single test site. In preferred embodiments, the multiple analytes (different influenza subtypes) are determined in the test devices.

The alternate pathways can include areas for mixing of reagents with test samples. For example, chambers can be used as areas of reagent addition. In addition, trapping devices may be included in the device pathway so as to remove fluid constituents above a certain size. For example, the devices of the present invention can include separators, e.g., to separate plasma or serum from whole blood. For example, a matrix of hydrophilic sintered porous material can have a red blood cell agglutinating agent applied to its surface. The matrix could be placed in the device anterior to the structures. The red blood cells in the whole blood sample become entrapped in the interstices of the matrix while substantially blood cell free serum or plasma passes through the matrix and is transported by capillary action to the structures part of the device. U.S. Pat. No. 4,933,092 is hereby completely incorporated by reference.

Automated

The antibodies of this invention can be readily adapted to automated immunochemistry analyzers. To facilitate automation of the methods of this invention and to reduce the turnaround time, a capture antibody in an immunoassay of this invention may be coupled to magnetic particles.

Antibody can be coupled to such magnetic beads by using commercially available technology as M-280 sheep anti-rabbit IgG coated Dynabeads. from Dynal, Inc., Lake Success, N.Y. (USA) and rabbit antibody to a target protein, or by using M-450 Tosylactivated Dynabeads from Dynal, Inc. and covalently coupling a relevant antibody thereto. Alternatively, an agent such as glutaraldehyde could be used for covalently coupling a subject antibody to a solid support, preferably magnetic beads. Representative coupling agents can include organic compounds such as thioesters, carbodiimides, succinimide esters, diisocyanates, glutaraldehydes, diazobenzenes and hexamethylene diamines.

A preferred automated/immunoassay system is the ACS: 180® Automated Chemiluminescence System (Bayer Corporation; Tarrytown, N.Y. and Medfield, Mass. (USA); including ACS: 180 PLUS System; ACS: 180 SE System; and ACS: CENTAUR® System). The ACS: 180® Automated Immunoassay System is described in Dudley, B. S., J. Clin. Immunoassay, 14 (2): 77 (Summer 1991). The system uses chemiluminescent labels as tracers and paramagnetic particles (PMP) as solid-phase reagents. The ACS: 180 system accommodates both competitive binding and sandwich-type assays, wherein each of the steps are automated. The ACS: 180 uses micron-sized paramagnetic particles that maximize the available surface area, and provide a means of rapid magnetic separation of bound tracer from unbound tracer without centrifugation. Reagents can be added simultaneously or sequentially. Other tags, such as an enzymatic tag, can be used in place of a chemiluminescent label, such as, acridinium ester. Luminescent signals would preferably be detected by a luminometer. Also preferred is the Bayer Immuno 1® Immunoassay System. Other exemplary automated devices that can be readily adapted to perform immunoassays utilizing antibodies of the invention are set forth in U.S. Pat. Nos. 5,807,522 and 6,907,722.

In another embodiment, anti-influenza antibodies of the invention can be incorporated into an automated multi-well platform to utilize immunoassay methods. The multi-well assay modules (e.g., plates) are adapted for induced luminescence-based assays inside one or more wells or chambers of a multi-well assay module (e.g., the wells of a multi-well assay plate). Multi-well assay plates may include several elements including, for example, a plate top, a plate bottom, wells, working electrodes, counter electrodes, reference electrodes, dielectric materials, contact surfaces for electrical connections, conductive through-holes electrically connecting the electrodes and contact surfaces, adhesives, assay reagents, and identifying markings or labels. The wells of the plates may be defined by holes in the plate top; the inner walls of the holes in the plate top may define the walls of the well. The plate bottom can be affixed to the plate top (either directly or in combination with other components) and can serve as the bottom of the well.

The multi-well assay modules (e.g., plates) may have any number of wells and/or chambers of any size or shape, arranged in any pattern or configuration, and be composed of a variety of different materials. Preferred embodiments of the invention are multi-well assay plates that use industry standard multi-well plate formats for the number, size, shape and configuration of the plate and wells. Examples of standard formats include 96-, 384-, 1536- and 9600-well plates, with the wells configured in two-dimensional arrays. Other formats include single well, two well, six well and twenty-four well and 6144 well plates. Preferably, the wells and/or chambers have at least one first electrode incorporated therein, and more preferably also include at least one second electrode. According to preferred embodiments, the wells and/or chambers have at least one working electrode incorporated therein, and more preferably also include at least one counter electrode. According to a particularly preferred embodiment, working, counter and, optionally, reference electrodes are incorporated into the wells and/or chambers. The assay plates are preferably flat, but may also be curved (not flat).

Moreover, one or more assay reagents may be included in wells, chambers and/or assay domains of an assay module (e.g., in the wells of a multi-well assay plate). For example, assay reagents including antibodies to different influenza virus or different epitopes of an influenza virus polypeptide can be utilized in different regions of the micro-titer palte(s). These assay reagents may be immobilized or placed on one or more of the surfaces of a well and/or chamber (preferably on the surface of an electrode, most preferably a working electrode) and may be immobilized or placed in one or more distinct assay domains (e.g. in patterned arrays of reagents immobilized on one or more surfaces of a well and/or chamber, preferably on working electrodes and/or counter electrodes, most preferably on working electrodes). The assay reagents may also be contained or localized by features within the well and/or chamber. For example, patterned dielectric materials may confine or localize fluids.

In one embodiment, an apparatus of the invention can be used to induce and measure luminescence in assays conducted in assay modules, preferably in multi-well assay plates. It may incorporate, for example, one or more photodetectors; a light tight enclosure; electrical connectors for contacting the assay modules; mechanisms to transport multi-well assay modules into and out of the apparatus (and in particular, into and out of light tight enclosures); mechanisms to align and orient multi-well assay modules with the photodetector(s) and with electrical contacts; mechanisms to track and identify modules (e.g. one or more bar code readers (e.g., one bar code reader for reading one side of a plate or module and another for reading another side of the plate or module); orientation sensor(s); mechanisms to make electrical connections to modules, one or more sources of electrical energy for inducing luminescence in the modules; and appropriate electronics and software.

The apparatus may also include mechanisms to store, stack, move and/or distribute one or more assay modules (e.g. multi-well plate stackers). The apparatus may advantageously use arrays of photodetectors (e.g. arrays of photodiodes) or imaging photodetectors (e.g. CCD cameras) to measure light. These detectors allow the apparatus to measure the light from multiple wells (and/or chambers) simultaneously and/or to image the intensity and spatial distribution of light emitted from an individual well (and/or chamber).

The apparatus can preferably measure light from one or more sectors of an assay module, preferably a multi-well assay plate. In some embodiments, a sector comprises a group of wells (and/or chambers) numbering between one and a number fewer than the total number of wells (and/or chambers) in the assay module (e.g. a row, column, or two-dimensional sub-array of wells in a multi-well plate). In preferred embodiments, a sector comprises between 4 percent and 50 percent of the wells of a multi-well plate. In especially preferred embodiments, multi-well assay plates are divided into columnar sectors (each sector having one row or column of wells) or square sectors (e.g., a standard sized multi-well plate can be divided into six square sectors of equal size). In some embodiments, a sector may comprise one or more wells with more than one fluid containment region within the wells. The apparatus, preferably, is adapted to sequentially induce ECL in and/or sequentially measure ECL from the sectors in a given module, preferably plate.

The apparatus may also incorporate microprocessors and computers to control certain functions within the instrument and to aid in the storage, analysis and presentation of data. These microprocessors and computers may reside in the apparatus, or may reside in remote locations that interact with the apparatus (e.g. through network connections).

Membranes/Surfaces

In various aspects of the invention, devices incorporating influenza virus antigens or anti-influenza virus antibodies comprise a surface or membrane. Various surfaces or membranes can provide a surface onto which an antibody or antigen is immobilized or disposed for utilization in various conventional immunoassay devices. As such, membranes can provide regions comprising test as well as control regions that utilize immunoreagents allowing visualization of a test result (e.g., whether a sample contains one or more viruses). In various embodiments, membranes having influenza virus antigens or anti-influenza virus antibodies disposed thereon are in turn disposed onto a solid substrate (e.g., lateral flow or dipstick device).

The membrane or surface to which antigen/antibody can be attached can comprise of a material including but not limited to cellulose, nitrocellulose, nylon, cationized nylon carrying a quaternary amino charge (Zeta probe), aminophenylthioether (APT) paper which is converted to DPT, the diazo derivative (this cannot be stained for use with enzyme detectable labels) or hydrophilic polyvinylidene difluoride (PVDF)-(available from Millipore, Billerica, Mass.). The term “nitrocellulose” is meant any nitric acid ester of cellulose. Thus suitable materials may include nitrocellulose in combination with carboxylic acid esters of cellulose. The pore size of nitrocellulose membranes may vary widely, but is frequently within about 5 to 20 microns, preferably about 8 to 15 microns. However, other materials are contemplated which are known to those skilled in the art. In some embodiments, the test region comprises a nitrocellulose web assembly made of Millipore nitrocellulose roll laminated to a clear Mylar backing. In another embodiment, the region comprising antigen/antibody (or “test region”) is made of nylon. In another embodiment, the test region is comprised of a material that can immobilize latex or other particles which carry a second reagent capable of binding specifically to an analyte, thereby defining a test zone, for example, compressed nylon powder, or fiber glass. In an occasional embodiment, the test region is comprised of a material that is opaque when in a dry state, and transparent when in a moistened state.

Test and Control Zones

Devices can include membranes/surfaces comprising test and control zones, constructed from any of the materials as listed above for the test region. Often the test and control zones form defined components of the test region. In one embodiment, the test and control zones are comprised of the same material as the test region. Frequently, the term “test region” is utilized herein to refer to a region in/on a device that comprises at least the test and control zones. In some embodiments the device utilizes a bibulous material but in some embodiments to provide non-bibulous flow, these materials may be treated with blocking agents that can block the forces which account for the bibulous nature of bibulous membranes. Suitable blocking agents include bovine serum albumin, methylated bovine serum albumin, whole animal serum, casein, and non-fat dry milk, as well as a number of detergents and polymers, e.g., PEG, PVA and the like. In some embodiments, the interfering sites on the untreated bibulous membranes are completely blocked with the blocking agent to permit non-bibulous flow there through. As indicated herein, the present disclosure envisages a test device with multiple test and control zones.

The test region generally includes one or more control zone that is useful to verify that the sample flow is as expected. Each of the control zones comprise a spatially distinct region that often includes an immobilized member of a specific binding pair which reacts with a labeled control reagent. In an occasional embodiment, the procedural control zone contains an authentic sample of the analyte of interest, or a fragment thereof. In this embodiment, one type of labeled reagent can be utilized, wherein fluid sample transports the labeled reagent to the test and control zones; and the labeled reagent not bound to an analyte of interest will then bind to the authentic sample of the analyte of interest positioned in the control zone. In another embodiment, the control line contains antibody that is specific for, or otherwise provides for the immobilization of, the labeled reagent. In operation, a labeled reagent is restrained in each of the one or more control zones, even when any or all the analytes of interest are absent from the test sample.

In some embodiments, solid supports can comprise patterned regions comprising antigen/antibody-binding matrix areas, which can be designed in any shape desired (e.g., square, oval, circle, vertical or horizontal lines). For example, the antigen-binding matrix areas disposed onto a solid support dipstick which may be made of materials such as plastic or Mylar. Through the use of this invention, it is possible to detect multiple anti-subtype H5 AIV antibodies in a single test through the incorporation of multiple matrix squares each containing different specific antigens at various positions on a single test strip, or on a single solid phase support dipstick.

In various embodiments, a device comprising antibodies of the invention to be utilized in an immunoassay can be included in a kit. The kit is formulated to contain the necessary reagents for the particular format of immunoassay being utilized. The kit may contain a dipstick and separate reagents utilized therewith, a lateral flow device on which is immobilized the antibodies necessary for the assay of multiple subtypes of influenza, or any conventional device with the necessary reagents. For example, through a process of sequentially dipping the dipstick through the series of reagents provided in the kit the presence or absence of particular anti-subtype influenza virus (e.g., H5 AIV antibody) or influenza virus antigen (e.g., H5 antigen) in a sample can be simply and quickly ascertained. Such kits would be suitable for use by experts and lay persons alike. The use of the present kit invention will permit the rapid serologic diagnosis of influenza virus (e.g., AIV) from body fluids such blood, urine, sputum, semen, feces, saliva, bile, cerebral fluid, nasal swab, urogenital swab, nasal aspirate, spinal fluid, etc.

In another embodiment, a solid phase support dipstick or lateral flow device is placed in a test tube or similar receptacle to which is added the specimen sample from the patient or animal suspected to be infected with influenza virus (e.g., AIV). The specimen sample is allowed to react with the bound antigens/antibodies on the dipstick. The dipstick is then removed and gently washed. The solid phase support dipstick is removed from the wash solution and placed in another tube containing highly diluted, affinity purified immunoglobulin, which is specific for the species that the sample was obtained from, conjugated to alkaline phosphatase or another suitable enzyme. The dipstick is then removed from the second-antibody solution and placed in a container of wash solution. Upon removal from the wash solution the dipstick is placed in a final tube of premixed chromogen solution or other suitable substrate solution. A positive reaction can be assessed by simple visual comparison of the control (upper spot) with the positive (lower spot). If the positive spot is darker than the control, then the test is considered positive. The enzymes which are covalently bound to the affinity purified immunoglobulin react with substrates which yield a color reaction product at the end of the enzyme-substrate reaction. In this way the presence of bound affinity purified immunoglobulin can be readily detected thereby indicating the presence of antibodies in the specimen sample that are specific for influenza virus (e.g., H5 antigen). The technology incorporates well known ELISA techniques, as also disclosed herein.

It is understood that the selection of appropriate enzymes and substrates and the appropriate reaction conditions would be known to one skilled in the art. These enzymes remain active after being conjugated to immunoglobulin molecules. Each enzyme-substrate pair reacts chemically to generate a colored reaction product. In addition there are alternative conjugates in which the enzyme and substrate are both conjugated into the affinity purified immunoglobulin solution but the enzyme and substrate only react to form the colored reaction product after the affinity purified immunoglobulin has bound to the has bound to the specimen antibody.

Of course, by utilizing different antibodies/antigens a sample can be readily screened for a panel of different virus types or subtypes. One of skill in art would understand that a variety of panels may be assayed via the immunoassays described herein. See, e.g., CURRENT PROTOCOLS IN IMMUNOLOGY (Coligan, John E., et. al., eds. 1999).

Arrays

Antibodies of the present invention can also readily be adapted for use in devices adapted for high throughput methods of detecting analytes, including detection of one or more influenza virus protein (e.g., H5) or of one or more of an anti-influenza virus antibody in a sample. Such methods include embodiments wherein antibodies are displayed in an array format that contains other antibodies, which target multiple different viruses such as other influenza subtypes (e.g., AIV). In other embodiments, antibodies can target the same antigen or target but specifically bind a different epitope on a given polyeptide.

In a yet a further embodiment of the invention, the array of antibodies comprises a substrate, and a plurality of patches arranged in discrete, known regions on the portions of the substrate surface wherein (i) each patch comprises antibodies immobilized on the substrate, wherein said antibodies of a given patch are capable of binding a particular viral expression product, fragment thereof, or host protein, such as an ant-viral antibody and (ii) the array comprises a plurality of different antibodies, each of which is capable of binding a different viral expression product, fragment thereof, or host protein, such as an ant-viral antibody.

The antibodies are preferably covalently immobilized on the patches of the array, either directly or indirectly. In most cases, the array will comprise at least about ten patches. In a preferred embodiment, the array comprises at least about 50 patches. In a particularly preferred embodiment the array comprises at least about 100 patches. In alternative preferred embodiments, the array of antibodies may comprise more than 10³, 10⁴ or 10⁵ patches.

The area of surface of the substrate covered by each of the patches is preferably no more than about 0.25 mm². Preferably, the area of the substrate surface covered by each of the patches is between about 1 μm² and about 10,000 μm². In a particularly preferred embodiment, each patch covers an area of the substrate surface from about 100 μm² to about 2,500 μm². In an alternative embodiment, a patch on the array may cover an area of the substrate surface as small as about 2,500 nm², although patches of such small size are generally not necessary for the use of the array.

The patches of the array may be of any geometric shape. For instance, the patches may be rectangular or circular. The patches of the array may also be irregularly shaped. The patches are optionally elevated from the median plan of the underlying substrate.

The distance separating the patches of the array can vary. Preferably, the patches of the array are separated from neighboring patches by about 1 μm to about 500 μm. Typically, the distance separating the patches is roughly proportional to the diameter or side length of the patches on the array if the patches have dimensions greater than about 10 μm. If the patch size is smaller, then the distance separating the patches will typically be larger than the dimensions of the patch.

In a particular embodiment of the array, the patches of the array are all contained within an area of about 1 cm² or less on the surface of the substrate. In one preferred embodiment of the array, therefore, the array comprises 100 or more patches within a total area of about 1 cm² or less on the surface of the substrate. Alternatively, a particularly preferred array comprises 10³ or more patches within a total area of about 1 cm² or less. A preferred array may even optionally comprise 10⁴ or 10⁵ or more patches within an area of about 1 cm² or less on the surface of the substrate. In other embodiments of the invention, all of the patches of the array are contained within an area of about 1 mm² or less on the surface of the substrate.

Typically, only one antibody is present on a single patch of the array. If more than one antibody is present on a single patch, all of the antibodies on that patch must share a common binding partner. For instance, a patch may comprise a variety of antibodies to the influenza viral protein (although, potentially, the antibodies may bind different epitopes on a given influenza virus). In preferred embodiments, the influenza viral protein/antigen is H5 and the influenza virus is AIV.

The arrays of the invention can have any number of a plurality of different antibodies. Typically the array comprises at least about ten different antibodies. Preferably, the array comprises at least about 50 different antibodies. More preferably, the array comprises at least about 100 different antibodies. Alternative preferred arrays comprise more than about 10³ different antibodies or more than about 10⁴ different antibodies. The array may even optionally comprise more than about 10⁵ different antibodies.

In one embodiment of the array, each of the patches of the array comprises a different antibody. For instance, an array comprising about 100 patches could comprise about 100 different antibodies. Likewise, an array of about 10,000 patches could comprise about 10,000 different antibodies. In an alternative embodiment, however, each different antibody is immobilized on more than one separate patch on the array. For instance, each different antibody may optionally be present on two to six different patches. An array of the invention, therefore, may comprise about three-thousand antibody patches, but only comprise about one thousand different antibodies since each different antibody is present on three different patches.

Typically, the number of different proteins which can be bound by the plurality of different antibodies on the array will be at least about ten. However, it is preferred that the plurality of different antibodies on the array is capable of binding a higher number of different proteins, such as at least about 50 or at least about 100. In still further preferred embodiments, the plurality of different antibodies on the array is capable of binding at least about 10³ proteins.

Use of the antibody arrays of this embodiment may optionally involve placing the two-dimensional array in a flow chamber with approximately 1-10 uL of fluid volume per 25 mm² overall surface area. The cover over the array in the flow chamber is preferably transparent or translucent. In one embodiment, the cover may comprise Pyrex or quartz glass. In other embodiments, the cover may be part of a detection system that monitors interaction between the antibodies immobilized on the array and protein in a solution such as a cellular extract. The flow chambers should remain filled with appropriate aqueous solutions to preserve antibody. Salt, temperature, and other conditions are preferably kept similar to those of normal physiological conditions. Samples in a fluid solution may be flushed into the flow chamber as desired and their interaction with the immobilized antibodies determined. Sufficient time must be given to allow for binding between the antibodies and their binding partners to occur. The amount of time required for this will vary depending upon the affinity of the antibodies for their binding partners. No specialized microfluidic pumps, valves, or mixing techniques are required for fluid delivery to the array.

Detection Means

As applicable to any device utilizing antibodies of the invention, a wide range of detection components are available for detecting the presence of binding partners. Detection may be either quantitative or qualitative. The invention array can be interfaced with optical detection methods such as absorption in the visible or infrared range, chemoluminescence, and fluorescence (including lifetime, polarization, fluorescence correlation spectroscopy (FCS), and fluorescence-resonance energy transfer (FRET)). Furthermore, other modes of detection such as those based on optical waveguides PCT Publication (WO 96/26432 and U.S. Pat. No. 5,677,196), surface plasmon resonance, surface charge sensors, and surface force sensors are compatible with many embodiments of the invention. Alternatively, technologies such as those based on Brewster Angle microscopy (BAM) (Schaaf et al., Langmuir, 3:1131-1135 (1987)) and ellipsometry (U.S. Pat. Nos. 5,141,311 and 5,116,121; Kim, Macromolecules, 22:2682-2685 (1984)) could be applied. Quartz crystal microbalances and desorption processes (see for example, U.S. Pat. No. 5,719,060) provide still other alternative detection means suitable for at least some embodiments of the invention array. An example of an optical biosensor system compatible both with some arrays of the present invention and a variety of non-label detection principles including surface plasmon resonance, total internal reflection fluorescence (TIRF), Brewster Angle microscopy, optical waveguide lighltmode spectroscopy (OWLS), surface charge measurements, and ellipsometry can be found in U.S. Pat. No. 5,313,264.

In some embodiments, the devices incorporating the antibodies of the invention can be incorporated into a system which includes a reader, particularly a reader with a built in computer, such as a reflectance and/or fluorescent based reader, and data processing software employing data reduction and curve fitting algorithms, optionally in combination with a trained neural network for accurately determining the presence or concentration of analyte in a biological sample. As used herein, a reader refers to an instrument for detecting and/or quantitating data, such as on test strips comprised in a test device utilizing antibodies of the invention. The data may be visible to the naked eye, but does not need to be visible. The methods include the steps of performing an immunoassay on a patient sample, reading the data using a reflectance and/or fluorescent based reader and processing the resultant data using data processing software employing data reduction. Preferred software includes curve fitting algorithms, optionally in combination with a trained neural network, to determine the presence or amount of analyte in a given sample. The data obtained from the reader then can be further processed by the medical diagnosis system to provide a risk assessment or diagnosis of a medical condition as output. In alternative embodiments, the output can be used as input into a subsequent decision support system, such as a neural network, that is trained to evaluate such data.

In various embodiments, the reader can be a reflectance, transmission, fluorescence, chemo-bioluminescence, magnetic or amperometry reader (or two or more combinations), depending on the signal that is to be detected from the device. Furthermore, some of the types of detection methods commonly used for traditional immunoassays which require the use of labels may be applied to the arrays of the present invention. These techniques include noncompetitive immunoassays, competitive immunoassays, and dual label, ratiometric immunoassays. These particular techniques are primarily suitable for use with the arrays of antibodies when the number of different antibodies with different specificity is small (less than about 100). In the competitive method, binding-site occupancy is determined indirectly. In this method, the antibodies of the array are exposed to a labeled developing agent, which is typically a labeled version of the analyte or an analyte analog. The developing agent competes for the binding sites on the antibodies with the analyte. The fractional occupancy of the antibodies on different-patches can be determined by the binding of the developing agent to the antibodies of the individual patches.

In the noncompetitive method, binding site occupancy is determined directly. In this method, the patches of the array are exposed to a labeled developing agent capable of binding to either the bound analyte or the occupied binding sites on the protein-capture agent. For instance, the developing agent may be a labeled antibody directed against occupied sites (ie., a “sandwich assay”). Alternatively, a dual label, ratiometric, approach may be taken where the antibody is labeled with one label and the second, developing agent is labeled with a second label (Ekins, et al., Clinica Chimica Acta., 194:91-114, 1990). Many different labeling methods may be used in the aforementioned techniques, including radioisotopic, enzymatic, chemiluminescent, and fluorescent methods. In some embodiments, fluorescent detection methods are preferred. Methods of detection include, but are not intended to be limited to, changes in color, light absorption, or light transmission, pH, conductivity, fluorescence, change in physical phase or the like.

Test samples may provide a detectable component of the detection system, or such components may be added. The components will vary widely depending on the nature of the detection system. One such detection method will involve the use of particles, where particles provide for light scatter or a change in the rate of flow. Particles may be, but are not intended to be limited to, cells, polymeric particles which are immiscible with a liquid system, latex particles, charcoal particles, metal particles, polysaccharides or protein particles, ceramic particles, nucleic acid particles, agglutinated particles or the like. The choice of particles will depend on the method of detection, the dispersability or the stability of the dispersion, inertness, participation in the change of flow, or the like. The binding of an analyte to a specific binding member at the capture site can be optionally detected by monitoring the pressure of the test sample in the device. For example, a pressure detector connected to the test sample entering and exiting the channel will allow the detection of pressure decreases caused by analyte binding which results in channel flow restriction.

For example, for quantifying the amount of detectable label present (e.g., antibody-conjugate), and thus the amount of antigen present, the procedure and apparatus of Hazelgrove et al., Anal. Biochem., 150:449-456, 1985) may be used. This procedure is based on a TV camera linked to a computer. The dots are displayed on a light box imaged by the TV camera, and digitized with a digitizing board (Techmar, Inc.). After digitizing, the computer will readout the position, width, height and relative area of each dot. Optical density (OD) measurements are plotted against absolute protein concentrations.

In another embodiment a device incorporating a Dot-ELISA test is used to detect a target protein directly from any sample. Therefore, antibodies of the invention can be utilized in a process comprising the steps:

(a) providing a solid support for performing a monoclonal antibody-based assay;

(b) applying to the solid support a sample suspected of containing an influenza virus;

(c) applying to the solid support a solution containing an organic acid, such as citric or lactic acid;

(d) applying to the solid support a solution containing a mucolytic agent and a detergent;

(e) contacting the solid support with a primary MAb, chimeric MAb, variant or fragment for a time sufficient to allow the MAb, chimeric MAb, variant or fragment and an H5 AIV protein to bind together to form an antigen-bound primary MAb;

(f) contacting the antigen-bound primary MAb with an enzyme labeled anti-MAb conjugate for a time sufficient to facilitate binding of the antigen-bound MAb to the conjugate; and

(g) applying a color reagent to the solid support, wherein the color reagent is catalyzed by the enzyme to develop a colored marking that allows visual detection of the presence of an H5 AIV protein or in the sample.

An exemplary Dot-ELISA method is disclosed in U.S. patent application 2006/0246429, which is incorporated by reference in its entirety.

In some embodiments a device or kit of the invention can be utilized in the field or point of care setting, where a chromogenic detection system, such as a system employing alkaline phosphatase may be used. For example, separate vials containing streptavidin-alkaline phosphatase conjugate in buffer, nitroblue tetrazolium (NBT), or 5-bromo-4-chloro-3-indolyl phosphate (BCIP), which are designed to be used in sequence to achieve the desired color may be supplied as components of a kit. With chromogenic detection systems results can be visualized by eye without the aid of any equipment. In one embodiment, a device can utilize alkaline phosphatase to quantitate the amount of AIV present. Such a device comprising alkaline phosphatase will give accurate quantitative results when used in conjunction with a densitometer.

In one embodiment, a kit for the detection of an H5 AIV protein or an anti-subtype H5 AIV antibody may comprise a detectable label such as streptavidin-alkaline phosphatase conjugate bound thereon; a reagent comprising nitroblue tetrazolium (NBTor 5-bromo-4-chloro-3-indolyl phosphate (BCIP); and a reference standard.

In any of the embodiments disclosed herein, the test sample may be derived from a source such as, but is not intended to be limited to, a physiological. Examples of test samples that can be administered to devices of the invention include samples suspected of containing an antigen or antibody, which are obtained from a non-human animal or a human subject, and include but are not limited to physiological fluids such as blood, serum, plasma, saliva, ocular lens fluid, cerebral spinal fluid, pus, exudate, milk, sweat, tears, ear flow, sputum, lymph, urine, egesta, secretion from oral or nasal cavities, tissues such as lung, spleen and kidneys, the liquid of the complete virus or lytic virus from chick embryo culture, and other samples suspected of containing an influenza virus protein or anti-influenza virus antibodies which are soluble or may be suspended in a suitable fluid. The test sample may be subject to prior treatment such as, but not intended to be limited to, extraction, addition, separation, dilution, concentration, filtration, distillation, dialysis or the like. Besides physiological fluids, other liquid test samples may be employed and the components of interest may be either liquids or solids whereby the solids are dissolved or suspended in a liquid medium. In one embodiment, a sample from the nasal cavity taken with a swab or other collection device is utilized in or with an immunoassay device. Devices will often contain a surface to which one or more antigen or antibody can be attached.

Treatment Methods and Pharmaceutical Compositions

The present invention provides a method of preventing or treating a disease associated with avian influenza virus infection in a subject comprising administering to said subject a pharmaceutically effective amount of the pharmaceutical composition comprising one or more monoclonal antibodies of the invention. The present invention also provides a pharmaceutical composition comprising one or more monoclonal antibodies of the invention or a pharmaceutically acceptable salt thereof.

The pharmaceutical composition of the invention may be administered to a subject through conventional administration routes, including without limitation, the oral, buccal, sublingual, ocular, topical, parenteral, rectal, intracisternal, intravaginal, intraperitoneal, intravesical, local (e.g., powder, ointment, or drop), or nasal routes.

Pharmaceutical compositions suitable for parenteral injection may comprise pharmaceutically acceptable sterile aqueous or nonaqueous solutions; dispersions, suspensions, or emulsions, and sterile powders for extemporaneous reconstitution into sterile injectable solutions or dispersions. Examples of suitable aqueous and nonaqueous carriers, vehicles, and diluents include water, ethanol, polyols (such as propylene glycol, polyethylene glycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

The pharmaceutical compositions of the invention may further comprise adjuvants, such as preserving, wetting, emulsifying, and dispersing agents. Prevention of microorganism contamination of the instant compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions may be affected by the use of agents capable of delaying absorption, for example, aluminum monostearate and gelatin.

Solid dosage forms for oral administration include capsules, tablets, powders, and granules. In such solid dosage forms, the active compound is admixed with at least one inert conventional pharmaceutical excipient (or carrier) such as sodium citrate or dicalcium phosphate, or (a) fillers or extenders, such as for example, starches, lactose, sucrose, mannitol, and silicic acid; (b) binders, such as for example, carboxymethyl-cellulose, alginates, gelatin, polyvinylpyrrolidone, sucrose, and acacia; (c) humectants, such as for example, glycerol; (d) disintegrating agents, such as for example, agar-agar, calcium carbonate, potato or tapioca starch, alginic acid certain complex silicates, and sodium carbonate; (e) solution retarders, such as for example, paraffin; (f) absorption accelerators, such as for example, quaternary ammonium compounds; (g) wetting agents, such as for example, cetyl alcohol and glycerol monostearate; (h) adsorbents, such as for example, kaolin and bentonite; and/or (i) lubricants, such as for example, talc, calcium stearate, magnesium stearate, solid polyethylene glycols, sodium lauryl sulfate, or mixtures thereof. In the case of capsules and tablets, the dosage forms may further comprise buffering agents.

Solid dosage forms may be formulated as modified release and pulsatile release dosage forms containing excipients such as those detailed above for immediate release dosage forms together with additional excipients that act as release rate modifiers, these being coated on and/or included in the body of the device. Release rate modifiers include, but are not limited to, hydroxypropylmethyl cellulose, methyl cellulose, sodium carboxymethylcellulose, ethyl cellulose, cellulose acetate, polyethylene oxide, xanthan gum, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients.

The pharmaceutical compositions of the invention may further comprise fast dispersing or dissolving dosage formulations (FDDFs) containing the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e., where the drug substance is insoluble, a fast dispersing dosage form may be prepared, and where the drug substance is soluble, a fast dissolving dosage form may be prepared.

Solid compositions of a similar type may also be employed as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well-known to one of ordinary skill in the art. They may also comprise opacifying agents, and can also be of such composition that they release the active compound(s) in a delayed, sustained, or controlled manner. Examples of embedding compositions that can be employed are polymeric substances and waxes. The active compound(s) can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, benzyl benzoate, propylene glycol, 1,3-butylene glycol, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the pharmaceutical composition can also include adjuvants, such as wetting agents, emulsifying and suspending agents, sweetening, flavoring, and perfuming agents. The pharmaceutical composition may further include suspending agents, such as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Pharmaceutical compositions of the present invention may also be configured for treatments in veterinary use, where a compound of the present invention, or a veterinarily acceptable salt thereof, or veterinarily acceptable solvate or pro-drug thereof, is administered as a suitably acceptable formulation in accordance with normal veterinary practice and the veterinary practitioner will determine the dosing regimen and route of administration which will be most appropriate for a particular animal.

One or more monoclonal antibodies of this invention may be used in combination with other anti-viral agents for prevention and/or treatment of diseases associated with H5 avian influenza virus infection. The monoclonal antibodies may be administered simultaneously, separately or sequentially with the other antiviral agents. Examples of other antiviral agents include without limitation ribavirin, amantadine, hydroxyurea, ribavirin, IL-2, IL-12 and pentafuside

Peptides Screening Methods and Peptides Recognized by the antibodies and Vaccines

The present invention provides a method of screening short peptides that simulate the epitopes recognized by the monoclonal antibodies of the invention. Furthermore, the present invention provides short peptides that simulate the epitopes recognized by the monoclonal antibodies of the invention. In one aspect, the present invention provides short peptides having the amino acid sequences set forth in SEQ ID NO: 64-68, 70-73, 74, 76, 78, 80, 82, 84, 86, 88, 90, 92, 94, and 96. These short peptides can bind to the monoclonal antibodies of the invention. Therefore, these short peptides have the same antigen specificity as the H5 hemagglutinin. The short peptides may be used to make a vaccine as the avian influenza virus subtype H5. The short peptides may also be used to detect the presence of anti-H5 antibodies.

In another aspect, the screening method of the invention comprises the steps of (i) culturing a peptide display library under conditions suitable for peptide expression; (ii) contacting the culture solution with monoclonal antibodies of the invention; (iii) selecting the phage clones that specifically bind to said monoclonal antibodies. The monoclonal antibodies used for the screening may include without limitation the monoclonal antibodies 8H5, 3C8, 10F7, 4D1 2F2, and/or 3G4. Examples 11-13 included herein describes in detail an assay that successfully screened short peptides that bind to the monoclonal antibodies of the invention using a peptide phage display libraries.

### EXAMPLES

The following Examples are further illustrative of the present invention, but are not to be construed to limit the scope of the present invention.

### Example 1

### Preparation of Monoclonal Antibodies Against the HA Antigen of Subtype H5 of Avian Influenza Virus

Preparation of Antigen.

Fertilized 9-day old chick embryos were inoculated with virus strain Ck/HK/Yu22/02 (H5N1) (referred to as “Yu22”) for 2 days at 30° C. The chick embryo supernatant was collected to obtain the amplified Yu22 virus. Live Yu22 virus were collected and inactivated with 0.03% formalin at 4° C. The HA antigen of the inactivated virus was detected and the titer of the inactivated virus was measured (please refer to the guidelines of WHO for the specific methods for determining the HA titer and detecting hemagglutination inhibition (HI). We chose the virus strain HA=1024, which was provided by the Microbiology Department of Hong Kong University).

Mice.

Six week old female Balb/c mice were purchased from the Anti-Cancer Center of Xiamen University. The mice were kept and tested in the center.

Production of Hybridoma.

We used standard in vivo immunization and PEG fusion methods to produce the hybridoma. For details of the methods please refer to Antibodies: A Laboratory Manual (Ed Harlow et al., Cold Spring Harbor Laboratory, 1988). The method was briefly described below.

Immunization of Mice. The above mentioned virus supernatant was mixed and emulsified with Complete Freund's adjuvant (CFA) in equal volume. The mixture was injected at multiple points of the muscles on the four legs of the mice at the dosage of 300 μl per mouse per injection. On the 15th and 29th day after the first immunization, the mixture was injected to the mice again at the same dosage as boosters. After the second booster, blood samples were taken from the mice to determine the inhibition potency by hemagglutination inhibition assay. When the potency reached 1:640, the mouse spleen was taken to carry out the fusion experiment. Another booster was injected 72 hr before the fusion experiment at the dosage of 50 μl per mouse through the caudal vein. 10 fusion plates were produced.

Fusion. The mouse spleen with the highest HI titer was fused with the mouse myeloma cells. First, the spleen was grinded to obtain the spleen cell suspension, then it was fused with the SP2/0 mouse myeloma cells in log phase growth at the ratio of 10 spleen cells versus 1 myeloma cell. The cells were fused together at the presence of PEG1500 for 1 minute. Then 100 ml of the fused cell solution was cultured in 10 96-well plates. The fusion medium was the RPMI1640 complete medium containing HAT and 20% FBS. The clones having the desired antigen specificity were screened by HI test, and stable monoclonal antibody producing cell lines were obtained after three rounds of cloning.

Screening of hybridoma. The fused cells were cultured on a 96-well cell plate for 10 days. The cell supernatant was extracted to do HI test. The wells containing positive clones were further cultured till the antibodies secreted by the cell line could stably inhibit agglutination between Yu22 virus strain and chicken blood.

Screening result. Six monoclonal antibody cell lines, 2F2, 3G4, 3C8, 4D01, 8H5 and 10F7, were obtained.

Culture of hybridoma. Stable cell lines capable of producing monoclonal antibodies were cultured first in a CO₂ incubator using 96-well plates, then transferred to 24-well plates, then transferred to a 50 ml cell culture flask for further amplification. The cells were collected from the cell flask and injected into a mouse abdominal cavity. Ascitic fluid was extracted from the mouse abdominal cavity after 7-10 days.

Purification of Monoclonal Antibodies.

The ascetic fluid was precipitated with 50% ammonium sulfate, then dialyzed with PBS at pH 7.2, purified with DEAE column by HPLC to obtain the purified monoclonal antibodies. The purity of the purified monoclonal antibody was determined with SDS-PAGE.

Virus HI Assay of the Monoclonal Antibodies

Thirty-four strains of H₅N₁ viruses from Vietnam, Indonesia, Malaysia, Thailand, Hong Kong, China Europe, etc. that belonged to different virus subtypes (Chen et al. PNAS, 103: 2845 (2006)) and 14 strains of non-H5 viruses (H1˜H13, Chicken NDV) were chosen to test the reactivity of the selected monoclonal antibodies with viruses using the HI assay. The results are shown in Tables 1 and 2. The results showed that all five strains of the H5 monoclonal antibodies had good specificity for the H5 viruses, and they did not react with the non-H5 viruses. As for reaction activity with the H5 virus strains, the reaction specificity varied among the different monoclonal antibodies. Except for the narrowest reaction spectrum of 3G4, the reaction spectra of the other four monoclonal antibodies with the viruses were all near or at 100%.

TABLE 1
Positive reaction rates between monoclonal antibodies and
H5 or non-H5 virus strains using HI assay

| | | H5 virus strain | Non-H5 virus strain |
|---|---|---|---|
| | | (Positive number/ | (Positive number/ |
| Monoclonal | Antibody | total virus | total virus |
| antibody | Subtype | number) | number) |
| 2F2 | IgG1 | 28/34 | 0/14 |
| 3G4 | IgG1 | 10/34 | 0/14 |
| 3C8 | IgG1 | 32/34 | 0/14 |
| 4D1 | IgG1 | 34/34 | 0/14 |
| 8H5 | IgG2a | 34/34 | 0/14 |
| 10F7 | IgM | 34/34 | 0/14 |

TABLE 2
HI Titer of Monoclonal Antibodies for 34 H5 Virus Strains

| H5N1 strains | Hamaglutinin inhibition titer of H5 mab agaisnt H5N1 | | | | | |
|---|---|---|---|---|---|---|
| according to | strains belong to different sublineage | | | | | |
| sublineage | 2F2 | 3C8 | 3G4 | 4D1 | 8H5 | 10F7 |
| GD1 | 12800 | 6400 | 12800 | 12800 | 12800 | 6400 |
| GD2 | 12800 | 12800 | 100 | 800 | 12800 | 12800 |
| GD3 | 12800 | 3200 | < | 12800 | 12800 | 12800 |
| YN1 | 800 | 1600 | < | 6400 | 3200 | 3200 |
| HN1 | 6400 | 3200 | < | 12800 | 12800 | 12800 |
| HN2 | 1600 | 800 | < | 3200 | 3200 | 1600 |
| IDN1.1 | 12800 | 3200 | < | 12800 | 12800 | 12800 |
| IDN2 | 800 | 400 | < | 800 | 1600 | 1600 |
| IDN3 | 1600 | < | < | 6400 | 3200 | 1600 |
| IDN4 | 12800 | 6400 | < | 12800 | 12800 | 12800 |
| IDN5 | 12800 | 6400 | < | 12800 | 6400 | 6400 |
| VTM1.1 | 12800 | 3200 | < | 3200 | 12800 | 3200 |
| VTM2 | 3200 | 1600 | < | 3200 | 6400 | 1600 |
| VTM3 | 12800 | 6400 | < | 12800 | 6400 | 12800 |
| VTM4 | 6400 | 800 | < | 400 | 1600 | 400 |
| VNM2.1 | 200 | < | < | 400 | 6400 | 3200 |
| MB1 | < | 400 | 3200 | 3200 | 3200 | 6400 |
| MB2 | 6400 | 3200 | < | 6400 | 6400 | 12800 |
| MIX1 | < | 1600 | 1600 | 12800 | 12800 | 12800 |
| MIX2 | < | 800 | 6400 | 12800 | 6400 | 12800 |
| MIX3 | 12800 | 400 | < | 12800 | 12800 | 12800 |

Note:
<, titer lower than 100.

Neutralization Test Between Monoclonal Antibodies and Viruses

The neutralization activities of the above mentioned monoclonal antibodies with H₅N1 viruses were detected by the micro-well neutralization test (Hulse-Post et al., PNAS, 102:10682-7 (2005)). The results in Table 3 demonstrate that monoclonal antibody 8H5 had good neutralization activities against all H5N1 virus strains.

TABLE 3
Monoclonal antibody titer for the H5N1 virus neutralization test.

| H5N1 strains | Neutralization titer of H5 mab against H5N1 | | | | | |
|---|---|---|---|---|---|---|
| according | strains belong to different sublineage | | | | | |
| to sublineage | 2F2 | 3C8 | 3G4 | 8H5 | 10F7 | 4D1 |
| GD1 | 12800 | 12800 | 200 | 12800 | 12800 | 12800 |
| GD2 | 12800 | 12800 | 12800 | 12800 | 12800 | 12800 |
| GD3 | 12800 | 200 | < | 12800 | 12800 | 12800 |
| YN1 | / | / | / | 6400 | 12800 | 12800 |
| HN1 | 12800 | 12800 | < | 12800 | 12800 | 12800 |
| HN2 | 12800 | 6400 | 100 | 6400 | 12800 | 12800 |
| IDN1.1 | 12800 | 6400 | < | 12800 | 12800 | 12800 |
| IDN2 | 12800 | 12800 | 100 | 6400 | 12800 | 12800 |
| IDN3 | / | / | / | 12800 | 12800 | 12800 |
| IDN4 | 12800 | 12800 | < | 12800 | 12800 | 12800 |
| IDN5 | 12800 | 6400 | < | 6400 | 12800 | 12800 |
| VTM1.1 | 1600 | 1600 | 100 | 1600 | 3200 | 1600 |
| VTM2 | 12800 | 6400 | < | 800 | 3200 | 3200 |
| VTM3 | 12800 | 12800 | 100 | 12800 | 12800 | 12800 |
| VTM4 | 12800 | 12800 | 12800 | 1600 | 200 | 1600 |
| VNM2.1 | 12800 | < | < | 3200 | 200 | 1600 |
| MB1 | / | / | / | 6400 | 12800 | 6400 |
| MB2 | 12800 | 12800 | 100 | 12800 | 12800 | 12800 |
| MIX1 | 100 | 100 | 100 | 12800 | 12800 | 12800 |
| MIX2 | 100 | < | 400 | 6400 | 12800 | 12800 |
| MIX3 | 12800 | 200 | < | 12800 | 12800 | 12800 |

Note:
/, no data;
<, titer lower than 100.

### Example 2

### Assembly of an HA Antigen Detection Kit for Subtype H5 Avian Influenza Virus (Using Enzyme Linked Immunosorbent Assay, ELISA)

The kit used the double-antibody sandwich method to detect the HA antigen of subtype H5 influenza virus in the sample. First, monoclonal antibodies against the HA antigen of subtype H5 influenza virus were pre-attached to the surface of the polythene micro-well plate in the kit box. The subtype H5 influenza virus containing HA antigen was pre-lysed and then added into the micro-well. The pre-attached monoclonal antibody would capture the HA antigens. Then enzyme-labeled monoclonal antibodies were added to the wells and bound to the antigens. At last, the binding results were visualized by the substrate color changes catalyzed by the enzyme. When the sample did not contain any influenza virus antigen or the virus was not subtype H5 influenza virus, the substrate would not change color. The samples could be animal waste, secretions of the mouth and nasal cavities, intact viruses, or lysed viruses cultured in chick embryo.

Preparation of the ELISA Plate

Monoclonal antibodies against the HA antigen of subtype H5 influenza virus were pre-attached to the surface of the polythene micro-well plate in the kit. The monoclonal antibodies were pre-attached to the plate by incubating in 10 nM phosphate buffer (PB, pH=7.4) overnight at 37° C., then washed with PBST (10 mM PBS+0.05% Tween 20), dried, and sealed with sealing solution (10 mMPBS+2% gelatin) for 2 hrs at 37° C. Then the plate was dried again and packaged in vacuum to produce the ELISA plate (8×12 well) of the detection kit.

Preparation of Other Components for the Detection Kit

Composition of the Virus Lysis Solution:
Lysis Buffer A (LB-A): 6% CHAPS+2% Tween-20+1% Tween-80;
Lysis Buffer B (LB-B): 100 mM PMSF, dissolved with isopropyl, and the final working concentration was 2 nM.
Lysis Buffer C (LB-C): 10 mM PBS, pH=7.4.

Enzyme-Labeled Reagent: Anti-HA monoclonal antibodies were labeled with Horse Radish Pecoxidase (HRP) and diluted to proper concentrations for use.

Positive control: The inactivated H5N1-Yu22 virus strain was used in a proper titer as the positive control.

Negative control: Lysis Buffer A was used as the negative control.

Developing Buffer A: Developing Buffer A: 13.4 g/L Na₂HPO₄.12H₂O+4.2 g/L citric acid aqueous solution +0.3 g/L Urea Peroxide.
Developing Bugger B: 0.2 mM/L 3,3′,5,5′-Tetramethylbenzidine (TMB)+20 mM/L dimethyl formamide (DMF).

Stop buffer: 2M concentrated sulfuric acid

Concentrated Washing Buffer: 20×PBST

Microplate Sealing Film: two sheets

Ziplock Bag: one

Instruction: one

Detection Procedure

Solution preparation: 50 ml concentrated washing buffer (20×) was diluted with distilled water or deionized water to 1000 ml for use.

Numbering: The samples were numbered according to the microplate sequence. 3 negative control wells, 2 positive control wells and 1 blank control well were set on every plate (sample and Enzyme-Labeled Reagent would not be added into the blank control well, and the remaining steps for the blank control well were the same as the other wells).

Sample Treatment and the Application

When the sample was liquid (including the original samples, chicken embryo culture samples, cell culture samples): An appropriate amount of the mixture of LB-A and LB-B at the ratio of 100 μl LB-A plus 4 μl LB-B was prepared. 100 μl sample was added into each well on the plate first then 100 μl of the prepared mixture of LB-A and LB-B was added.

When the sample was dry swab sample: 1 ml LB-A, 40 μl LB-B and 1 ml PBS were mixed together and added into a sample tube. The sample was agitated and dissolved in the solution and was incubated for 30 min at room temperature. The mixture was agitated again for suspension and was centrifuged for 5 min at 6000 rpm. The supernatant was extracted and 100 μl of the supernatant was added as a sample to a well for testing.

When the sample was dry animal waste: 1 ml LB-A, 40 μl LB-B and 1 ml PBS were mixed together and added into a sample tube. The dry animal waste was suspended in the solution to produce a 10% (w/v) sample suspension. The sample was dissolved after agitating and was incubated for 30 min at room temperature. The mixture was agitated again for suspension and was centrifuged for 5 min at 6000 rpm. Then the supernatant was extracted and 100 μl of the supernatant was added as a sample to a well for testing.

Negative and positive control wells should be included for every detection experiment. 100 μl control solution should be added to each control well.

Incubation: The plate was sealed with microplate sealing film and the plate shaker was set at high or moderate speed to shake the plate for 60 min at room temperature (25˜28° C.).

Washing: The sealing film was carefully removed and the plate was washed for 5 times with the plate washing machine and then dried.

Add enzyme: 100 μl Enzyme-Labeled Reagent was added into each relevant well.

Incubation: The plate was sealed with sealing film and incubated for 30 min at 37° C.

Repeat Step 6.

Color reaction: Developing Buffer A and Developing Buffer B at 50 μl each were added into each well. The plate was shaken gently to mix them well and the mixture was kept away from light for color reaction for 30 min at 37° C.

Detection: A drop of Stop Buffer (50 μl) was added into each well and gently shaken to mix well. The OD values of each well were determined with plate analyzer at single wavelength of 450 nm (blank control was needed) or dual-wavelength of 450 nm/630 nm.

Result Assessment

a) Normal range of negative control: Under normal conditions, OD value of the negative well was no more than 0.1 (if the OD value of all the negative control well was more than 0.1, it should be discarded; if the OD values of all the negative control wells were more than 0.1, the experiment should be repeated. If the OD values of the negative control wells were less than 0.03, then the OD value should be regarded as 0.03).

b) Normal range of the positive control: Under normal conditions, the OD value of the positive control should be no less than 0.5.

c) Determination of the CUTOFF value (low case): 0.15 was added to the average OD values of the negative control wells.

d) Determination of the Positive Reaction: If the OD value≧the CUTOFF, it was a positive reaction for HA antigen of H5 avian influenza virus.

e) Determination of the Negative Reaction: If the OD value<the CUTOFF, it was a negative reaction for HA antigen of H5 avian influenza virus.

Detection Test of Clinical Samples

The kit was used to detect all kinds of H5 and non-H5 virus samples and the results are shown in Table 4. It demonstrated that the kit had very good detection sensitivity and specificity.

TABLE 4
Detection of H5 and non-H5 virus samples with H5 antigen ELISA
method

| Sample type | H5 | Non-H5 |
|---|---|---|
| Human swab | — | 1^{a}/200^{b} |
| Chicken swab | 144^{a}/300^{b} | 1^{a}/87^{b} |
| Chicken embryo culture | 38^{a}/38^{b} (≦1 HA titer) | 0^{a}/46^{b} (≧256 HA titer) |

—, not determined;
^{a}positive number of samples;
^{b}total number of samples tested; HA titer is a standard unit for evaluation of the titer of influenza virus.

### Example 3

### Assembly of a Detection Kit (ELISA) for Anti-HA Antibody of Subtype H5 Avian Influenza Virus

The kit used the competition method to detect the specific anti-HA antibody in blood serum samples. First, monoclonal antibodies against the HA antigen of subtype H5 avian influenza virus were pre-attached to the surface of the microwell plate in the kit. Next, recombinantly expressed HA antigens of subtype H5 avian influenza virus were attached to the pre-attached antibodies. When the serum sample and the enzyme-labeled monoclonal antibodies were added to the plate, the specific antibodies in the sample and the enzyme-labeled monoclonal antibodies would compete for binding to the antigens on the plate. If the serum sample could noticeably inhibit the binding of enzyme-labeled monoclonal antibodies to the HA antigens, it would demonstrate that the sample contained the specific anti-HA antibodies. If the sample did not contain the anti-HA antibodies or it was not antibodies against subtype H5 avian influenza virus, the reaction between enzyme-labeled monoclonal antibodies and antigens would not be inhibited.

Preparation of the ELISA Plate

Monoclonal antibodies against the HA antigen of subtype H5 avian influenza virus were pre-attached to the surface of the polythene microwell plate in the kit. The monoclonal antibodies were attached by incubating in 10 nM phosphate buffer (PB, pH=7.4) overnight at 37° C., washed with PBST (10 mM PBS+0.05% Tween 20) once, dried, then sealed with the sealing solution (10 mMPBS+2% gelatin) for 2 hrs at 37° C. It was dried again for the attachment of the recombinant HA antigens. The recombinant HA antigens were diluted in 10 mM PBS (pH=7.4), then 100 μl of the diluted solution was added into each well of the antibody pre-attached plate, which was incubated for 2 hrs at 37° C., washed once, sealed for 2 h, and then packaged in a vacuum to become the finished ELISA plate (8×12 well) of the kit.

Preparation of Other Components of the Kit

a) Enzyme-Labeled Reagent: The monoclonal antibodies were labeled against the HA antigen of H5 influenza virus with HRP. The labeled antibodies were stored in a proper dilution to make the Reagent.

b) Positive control: A proper concentration of monoclonal antibody against the HA antigen of H5 influenza virus was used as the positive control.

c) Negative control: 100% calf serum (NBS) was uses as the negative control.

d) Developing Buffer A: 13.4 g/L Na₂HPO₄.12H₂O+4.2 g/L citric acid aqueous solution +0.3 g/L Urea Peroxide

e) Developing Bugger B: 0.2 mM/L 3,3′,5,5′-Tetramethylbenzidine (TMB)+20 mM/L dimethyl formamide (DMF)

f) Stop buffer: 2M concentrated sulfuric acid.

g) Concentrated Washing Buffer: 20×PBST.

h) Microplate Sealing Film: two pieces

i) Ziplock Bag: one

j) Instruction: one

Detection Procedure

a) Liquid preparation: 50 ml concentrated Washing Buffer (20×) was diluted with distilled water or deionized water to 1000 ml for further use.

b) Numbering: The sample was numbered according to the microplate sequence. 3 negative control wells, 2 positive control wells and 1 blank control well were set on every plate (sample and the Enzyme-Labeled Reagent would not be added into the blank control well, and the remaining steps for the controls were the same as for the samples).

c) Sample application: 50 μl sample, negative control and positive control were add into relevant wells.

d) Adding enzyme: 50 μl Enzyme-Labeled Reagent was added into the relevant wells.

e) Incubation: The plate was sealed with sealing film after the solution in the well was mixed, then it was incubated for 60 min at 37° C.

f) Washing: The sealing film was carefully removed, and the plate was washed 5 times with the plate washer and then dried.

g) Color reaction: Developing Buffer A and Developing Buffer B at 50 μl each were added into each well and gently shaken to mix well. The mixture was kept away from light for the color reaction for 15 min at 37° C.

h) Detection: A drop of stop buffer (50 μl) was added into each well and gently shaken to mix well. The OD values of each well were determined with plate analyzer at single wavelength of 450 nm (blank control was needed) or dual-wavelength of 450 nm/630 nm.

Result Assessment

a) Normal range of negative control: Under normal conditions, OD value of the negative control was no less than 0.1.

b) Normal range of the positive control: Under normal conditions, the OD value of the positive control should be no more than 0.1.

c) Determination of the CUTOFF value Cutoff value=half of the average OD values of the negative wells.

d) Determination of the Positive Reaction: If the sample's OD value<the CUTOFF, the sample was positive for anti-HA antibodies.

e) Determination of the Negative Reaction: If the sample's OD value≧the CUTOFF, the sample was negative for anti-HA antibodies.

Detection Test of Clinical Samples

The H5 antibody kit was used to test human and chicken serum samples. Table 5 shows that the kit had very good detection sensitivity and specificity.

TABLE 5
Detection of serum samples with anti-H5 antibody ELISA method

| Sample type | H5 | Non-H5 |
|---|---|---|
| Human serum | — | 0^{a}/1200^{b} |
| Chicken serum | 49^{a}/50^{b} | 0^{a}/24^{b} |

—, not determined;
^{a}positive number of samples;
^{b}total number of samples tested.

### Example 4

### Assemble of HA Antigen Detection Kit for Subtype H5 Avian Influenza Virus (Colloid Gold Labeling Method)

The test paper was a new generation of diagnostic reagent using the colloid gold immunochromotography technique. The samples which could be tested included animals waste, secretions of the mouth and nasal cavities, intact virus or lysed virus cultured in chicken embryo, etc. The product was delicately designed for one-time use only, and is simple, safe, reliable and creates no pollution. It contained quality control itself and needed no additional reagents. The result displayed was clear. The reaction was rapid, and the total operation needed only 30 min.

The test paper contained anti-HA monoclonal antibodies in the testing area on the nitrocellulose filter, and goat anti-mouse IgG in the control area. When testing, the H5 influenza virus in the sample and the colloid gold labeled anti-HA monoclonal antibody (Ab-Au) formed a complex (Ag-Ab-Au), the complex moved along the membrane because of the laminar separation effect, and it could form double antibody sandwich immunocomplex with the anti-HA monoclonal antibody in the testing area. If it was a positive sample, it could form red lines in the testing area and the control area, respectively; if it was a negative sample, it could only form a red line in the control area.

Preparation of the Test Paper in the Kit: the Test Paper was Prepared Using Standard Methods.

The kit contained test paper, lysis buffer and instruction.

Operation Procedure

a) Sample treatment and application

i) When the sample was liquid (including original samples, chicken embryo culture samples, cell culture samples):

An appropriate amount of lysis mixture of LB-A and LB-B in the ratio of 100 μl LB-A versus 4 μl LB-B was prepared. 100 μl sample was added into each well of the plate then 70 μl of the lysis mixture was added to each well.

ii) When the sample was dry swab sample:

1 ml LB-A, 40 μl LB-B and 1 ml PBS were mixed together and added into a sample tube. The sample was agitated and dissolved then incubated for 30 min at room temperature. The mixture was agitated again for re-suspension and centrifuged for 5 min at 600 rpm. The supernatant was extracted and 70 μl of the supernatant was added to each well for detection.

iii) When the sample was dry animal waste:

1 ml LB-A, 40 μl LB-B and 1 ml PBS were mixed together and added into a sample tube. The dry animal waste was suspended in the mixture to make a 10% (w/v) sample suspension. After agitating, the sample was dissolved and incubated for 30 min at room temperature. The mixture was agitated for re-suspension then centrifuged for 5 min at 6000 rpm. The supernatant was extracted and 70 μl of the supernatant was added to each well for testing.

b) 70 μl sample was added gradually at the sample loading site, and then placed at room temperature.

Result assessment: The results were observed within 30 min.

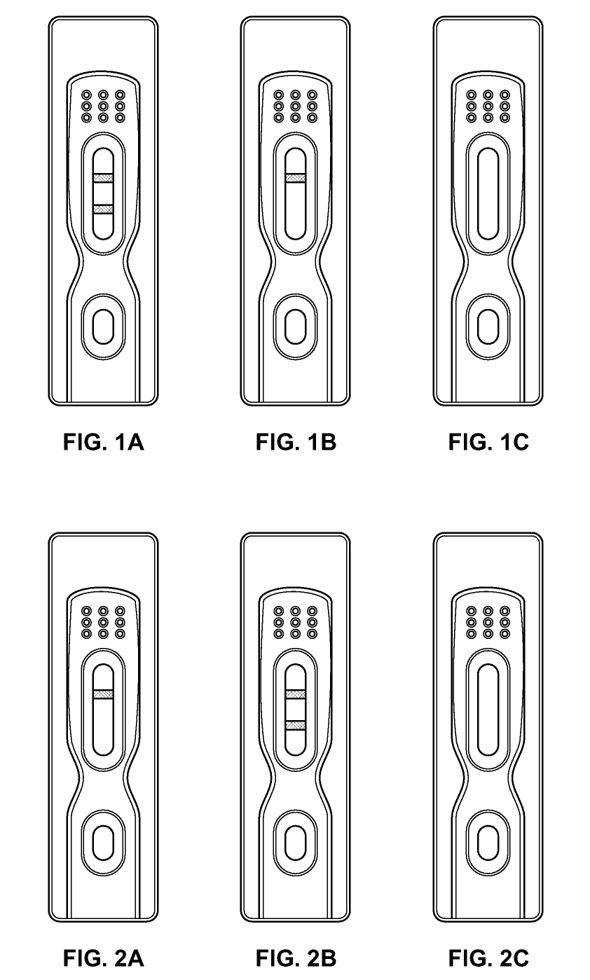

It was positive when two red lines appeared, negative if only the quality control line appeared, and invalid if no red line appeared. FIG. 1 showed the results of detection using a test paper.

### Example 5

### Assemble of Anti-HA Antibody Detecting Kit for Subtype H5 Avian Influenza Virus (Colloidal Gold Labeling Method)

The test paper contained anti-HA monoclonal antibodies in the testing area on the nitrocellulose filter, and goat anti-mouse IgG in the control area. There were freeze dry anti-HA monoclonal antibody labeled with colloid gold and recombinantly expressed HA antigen of subtype H5 influenza on the glass fiber. The competition method was applied to detect subtype H5 avian influenza virus anti-HA antibody in the sample. If there were anti-HA antibodies in the sample, it would compete with the colloid gold labeled anti-HA monoclonal antibody, thus to block the formation of the complex of colloid gold labeled antibody and HA antigen, and color could not be developed; if it was negative, the complex would be formed and color would develop.

Preparation of the Test Paper of the Kit: the Test Paper was Prepared Using Standard Methods.

Composition of the kit: The kit contained test paper and instruction.

Detection Procedure

A test paper was taken and 70 μl serum sample was added gradually on the sample loading site, and then placed at room temperature. The results were observed within 30 min. The result would be invalid if the time surpassed 30 min.

Result Assessment

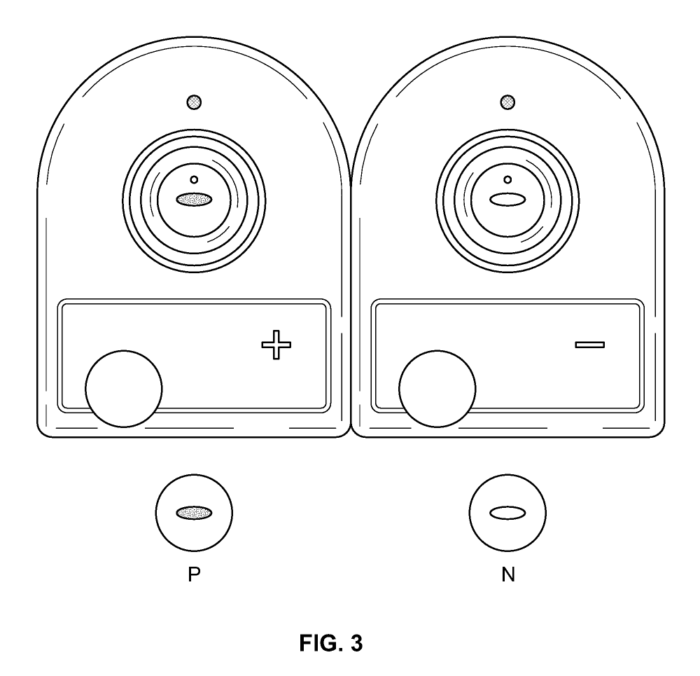

It was positive when only the quality control line appeared, negative if two red lines appeared, and invalid if no red line appeared. FIG. 2 showed the results of such a test.

### Example 6

### Assembly of the HA Antigen Dot-ELISA Detection Kit for Subtype H5 Avian Influenza Virus

The test paper was pre-coated with anti-H5 (HA) monoclonal antibodies in the testing area on the nitrocellulose filter, and the goat anti-mouse IgG in the control area. After the lysed sample containing subtype H5 influenza virus was added, the pre-coated monoclonal antibodies captured them, and the antigen-antibody complexes (Ab-Ag) were formed, then the enzyme-labeled monoclonal antibodies (Ab-HRP) were added to bound with the Ab-Ag complexes, and antibody-antigen-enzyme labeled antibody complexes (Ab-Ag-Ab-HRP) were formed, then the results were observed through enzyme catalyzed substrate coloration. When there was subtype H5 avian influenza virus, substrate coloring would appear in both the testing area and the control area. When there was no subtype H5 of avian influenza virus, the testing region would not show color, only a coloration spot would form in the control area.

Preparation of the Infiltration Detection Device

Nitrocellulose membrane and water absorbing filter paper were put on a flat bottom support. A matching cover was put on top of the bottom support wherein the cover has an opening in the middle. A flow control unit with a shape matching the opening on the cover was fit into the opening. The flow control unit has two holes for loading the sample and the control, respectively. The bottom of the flow control unit was pressed tightly against the bottom support to restrict sample flow on the nitrocellulose membrance and the filter paper. Anti-H5 (HA) monoclonal antibody was coated onto the test area and goat anti-mouse IgG was coated onto the control area in the flow control unit. The test paper was air dried for 1 h, and packaged in vacuum to become the infiltration detection device.

Composition of the Kit

The kit contained the following items:

a) Infiltration detection device

b) Sample processing device: A bottle with a filter cap screwed onto it; the filter cap contained a filter in the middle to allow solution to filter through it.

c) Enzyme-Labeled Reagent: Anti-H5 (HA) monoclonal antibodies with HRP were labeled and diluted to proper concentration to produce the Enzyme-Labeled Reagent.

d) Lysis Buffer: 3% NP40+1% Triton X-100+40 mM PBS, pH=7.4.

e) Washing Buffer: 2% Triton X-100+20 mM EDTA+0.25% Tween 20+0.1% Proclin 300+150 mM Nacl+5 mM PBS, pH=7.4.

f) Developing Buffer: 3,3′,5,5′-Tetramethylbenzidine (TMB) Liquid Substrate System for Membranes.

g) Stop buffer: 50 mM citric acid in H₂O.

h) Instruction

Detection Procedure

a) Sample processing and application:

200 μl sample was added into each sample processing unit. 8 drops of lysis buffer were put in and mixed completely. Then all of the lysed sample was squeezed out of the sample processing unit through the filter cap and loaded into the detection well. After the sample was completely absorbed (about 25 min), the flow control unit was removed.

b) Washing: 5 drops of washing buffer were added and then were left to absorb completely.

c) Add enzyme: 4 drops of Enzyme-Labeled Reagent were added; after all the enzyme was absorbed, the sample was left to react for 2 min.

d) Washing: Wash was made for two times. 8 drops of washing buffer were added at the first wash and 5 drops were added again after the washing buffer of the first wash were absorbed then they were left to absorb completely.

e) Coloration: 2 drops of developing buffer were added. The results were observed 2 min after the liquid was absorbed completely. The results would have no clinical significance after 5 min. FIG. 3 showed a schematic diagram for results assessment.

Clinical sample detection. H5 quick detection kit was used to detect clinical sample and the results are shown in Table 6. The results demonstrated that the kit had very good sensitivity and specificity.

TABLE 6
Detection of clinical virus samples by H5 antigen immunofiltration
method.

| Sample type | H5 | Non-H5 |
|---|---|---|
| Human swab | — | 1^{a}/36^{b} |
| Chicken swab | 55^{a}/70^{b} | 2^{a}/137^{b} |
| Chicken embryo culture | 38^{a}/38^{b} | 0^{a}/50^{b} |

—, not determined;
^{a}positive number of samples;
^{b}total number of samples tested.

### Example 7

### Isolation of the Light Chain and Heavy Chain Genes of the Monoclonal Antibodies

10⁷ hybridoma cells were cultured in semi-adherent culture flask. The cells adhering to the flask walls were blown away from walls to suspend them. The cells were transferred to another 4 ml centrifuge tube, centrifuged at 1500 rpm for 3 min. The precipitated cells were collected and suspended again in 100 μl PBS (pH=7.45), and then transferred to another 1.5 ml centrifuge tube. 800 μl Trizol (Roche, Germany) was added to the tube, gently mixed, and incubated for 10 min. 200 μl chloroform was added and agitated for 15 sec, incubated for 10 min, centrifuged at 12000 rpm at 4° C. for 15 min. The top layer of the liquid was transferred to another 1.5 ml centrifuge tube. Equal volume of Isopropanol of the same volume was added to the tube, mixed and incubated for 10 min. The mixture was centrifuged at 12000 rpm at 4° C. for 10 min. The supernatant was discarded and 600 μl 75% ethanol was added to wash the debri. The mixture was centrifuged at 12000 rpm at 4° C. for 5 min. The supernatant was discarded and the remainder of the mixture was precipitated at 60° C. and vacuumed for 5 min. The transparent sediments were dissolved in 70 μl DEPC H₂O. The solution was divided into two tubes. 1 μl primer for reverse transcription was added into each tube. In one tube, the primer was MVJkR (5′-CCg TTT(T/g) AT (T/C) TC CAg CTT ggT (g/C) CC-3′). It was used to amplify the genes in variable region of the light chain. The primer in another tube was MVDJhR (5′-C ggT gAC Cg (T/A)ggT (C/g/T) CC TTg (g/A) CC CCA-3′), which was used to amplify the genes in variable region of the heavy chain. 1 μl dNTP (Shanghai Sangon) was added into each tube. The tube was put in waterbath at 72° C. for 10 min. Then the tube was put immediately into an ice bath for 5 min. 10 μl 5× reverse transcription buffer, 1 μl AMV (10 u/μl, Pormega) and 1 μl Rnasin (40 u/μl, Promega) were added to the tube and mixed. Reverse transcription of the RNA to cDNA was carried out at 42° C.

Polymerase chain reaction (PCR) was used to amplify the light chain and heavy chain variable regions of the antibody gene. A set of primers were synthesized at Shanghai Bioasia. Other two primers, MVJkR and MVDJhR, were designed and synthesized at Shanghai Bioasia. The two cDNA molecules synthesized by the reverse transcription described above were used as templates. The conditions for the PCR reactions were: 94° C. for 5 min, 94° C. for 40 sec, 53° C. for 1 min, 72° C. for 50 sec, repeat for 35 cycles, 72° C. for 15 min. The PCR products were collected and cloned into pMD 18-T vectors. The PCR products were sequenced by Shanghai Bioasia and the sequences of the variable regions were confirmed through BLAST sequence comparison. The corresponding amino acid sequences were deduced from the gene sequences.

Using the above method, the variable region genes of the antibody were cloned from the hybridoma cell lines of six strains of avian influenza monoclonal antibodies, and the corresponding amino acid sequences were deduced. The primer sequences are shown in Table 7. The serial numbers of the variable region nucleic acids of the six strains of monoclonal antibodies and the corresponding amino acids are shown in Table 8. The Complementary Determinant Regions (CDRs) are shown in Table 9.

TABLE 7

Primer sequences for the amplification of
the variable region genes of monoclonal
antibodies of avian influenza virus.

| Variable region | | |
|---|---|---|
| of monoclonal | Primer | |
| antibody strains | Name | Primer Sequence |
| 8H5 Vh | MuIgVh5′-E2 | 5′-AT gg(A/g) ATg |
| | | gA(C/g) C(g/T)(g/T) |
| | | I(A/g)T CTT T(A/C)T |
| | | CT-3′ |
| | | (SEQ ID NO: 98) |
| 8H5 Vk | MuIgkVl5′-G1 | 5′-AT ggA TTT (A/T) |
| | | CA (A/g)gT gCA gAT |
| | | T(A/T)T GAg CTT-3′ |
| | | (SEQ ID NO: 99) |
| 3C8 Vh | MuIgVh5′-C2 | 5′-AT gg(A/C/g) TTg |
| | | g(C/g)T gTg gA(A/C) |
| | | CTT gC(C/T) ATT |
| | | CCT-3′ |
| | | (SEQ ID NO: 100) |
| 3C8 Vk | MuIgkVl5′-G3 | 5′-AT ggT (C/T)CT |
| | | (C/T)AT (A/C/g)TT |
| | | (A/g)CT gCT gCT ATg |
| | | g-3′ |
| | | (SEQ ID NO: 101) |
| 10F7 Vh | MuIgVh5′-B1 | 5′-ATg (A/g)AA |
| | | Tg(C/g) A(C/g)C Tgg |
| | | gT(C/T) (A/T)T(C/T) |
| | | CTC TT-3′ |
| | | (SEQ ID NO: 102) |
| 10F7 Vk | MuIgkVl5′-F2 | 5′-AT ggT (A/g)TC |
| | | C(A/T)C A(C/g)C TCA |
| | | gTT CCT Tg-3′ |
| | | (SEQ ID NO: 103) |
| 4D1 Vh | MuIgVh5′-B1 | 5′-ATg (A/g)AA |
| | | Tg(C/g) A(C/g)C Tgg |
| | | gT(C/T) (A/T)T(C/T) |
| | | CTC TT-3′ |
| | | (SEQ ID NO: 104) |
| 4D1 Vk | MuIgkVl5′-D1 | 5′-ACT AgT CgA CAT |
| | | gAg g(A/g)C CCC TgC |
| | | TCA g(A/T)T T(C/T)T |
| | | Tgg I(A/T)T CTT-3′ |
| | | (SEQ ID NO: 105) |
| 3G4 Vh | MuIgVh5′-E2 | 5′-AT gg(A/g) ATg |
| | | gA(C/g) C(g/T)(g/T) |
| | | I(A/g)T CTT T(A/C)T |
| | | CT-3′ |
| | | (SEQ ID NO: 106) |
| 3G4 Vk | | |
| 2F2 Vh | MuIgVH5′-C1 | 5′-CGA CAT GGC TGT |
| | | C(C/T)T |
| | | (A/G)G(C/G/T) GCT |
| | | G(C/T)T C(C/T)T |
| | | CTG-3′ |
| | | (SEQ ID NO: 107) |
| 2F2 Vk | MuIgkVL5′-G2 | 5′-CGA CAT GGT |
| | | (C/T)CT (C/T)AT |
| | | (A/C/G)TC CTT GCT |
| | | GTT CTG G-3′ |
| | | (SEQ ID NO: 108) |

TABLE 8
Serial numbers of variable region nucleic acids of six strains of
monoclonal antibodies and the corresponding amino acids.

| Monoclonal | | | | |
|---|---|---|---|---|
| antibody | Vh nucleic acid | Vh amino acid | Vk nucleic acid | Vk amino acid |
| name | sequence | sequence | sequence | sequence |
| 8H5 | SEQ ID NO: 1 | SEQ ID NO: 2 | SEQ ID NO: 3 | SEQ ID NO: 4 |
| 3C8 | SEQ ID NO: 5 | SEQ ID NO: 6 | SEQ ID NO: 7 | SEQ ID NO: 8 |
| 10F7 | SEQ ID NO: 9 | SEQ ID NO: 10 | SEQ ID NO: 11 | SEQ ID NO: 12 |
| 4D1 | SEQ ID NO: 16 | SEQ ID NO: 17 | SEQ ID NO: 18 | SEQ ID NO: 19 |
| 3G4 | SEQ ID NO: 20 | SEQ ID NO: 21 | SEQ ID NO: 22 | SEQ ID NO: 23 |
| 2F2 | SEQ ID NO: 24 | SEQ ID NO: 25 | SEQ ID NO: 26 | SEQ ID NO: 27 |

TABLE 9
Six strains of monoclonal antibody CDRs amino acid sequence.

| | | |
|---|---|---|
| Monoclonal | Antibody heavy chain CDRs | Antibody light chain CDRs |
| antibody | amino acid sequence | amino acid sequence |

| | | | | | | |
|---|---|---|---|---|---|---|
| strains | CDR1 | CDR2 | CDR3 | CDR1 | CDR2 | CDR3 |

| | | | | | | |
|---|---|---|---|---|---|---|
| 8H5 | GYTFSNYW | ILPGSDRT | ANRYDGYYFGLDY | SSVNF | YSS | QHFTSSP |
| | (SEQ ID | (SEQ ID | (SEQ ID | (SEQ ID | (SEQ ID | (SEQ ID |
| | NO: 28) | NO: 29) | NO: 30) | NO: 31) | NO: 32) | NO: 33) |
| 3C8 | GYSFTNYG | INTHTGEP | ARWNRDAMDY | ESVDSSDNSL | TAS | QQSIGDPPYT |
| | (SEQ ID | (SEQ ID | (SEQ ID | (SEQ ID | (SEQ ID | (SEQ ID |
| | NO: 34) | NO: 35) | NO: 36) | NO: 37) | NO: 38) | NO: 38) |
| 10F7 | GYTFTSYW | IDPSDSYT | ARGGTGDFHYAMDY | QGISSN | HGT | QYVQFPYT |
| | (SEQ ID | (SEQ ID | (SEQ ID | (SEQ ID | (SEQ ID | (SEQ ID |
| | NO: 40) | NO: 41) | NO: 42) | NO: 43) | NO: 44) | NO: 45) |
| 4D1 | GYTFTSYW | IDPSDSFT | ARGGPGDFRYAMDY | QGISSN | HGT | VQYVQFPYT |
| | (SEQ ID | (SEQ ID | (SEQ ID | (SEQ ID | (SEQ ID | (SEQ ID |
| | NO: 46) | NO: 47) | NO: 48) | NO: 49) | NO: 50) | NO: 51) |
| 3G4 | GYTFTDYA | INTDYGDT | ARSDYDYYFCGMDY | | | |
| | (SEQ ID | (SEQ ID | (SEQ ID | (SEQ ID | (SEQ ID | (SEQ ID |
| | NO: 52) | NO: 53) | NO: 54) | NO: 55) | NO: 56) | NO: 57) |
| 2F2 | GFSLTGYG | IWAEGRT | AREVITTEAWYFDV | QSISDY | YAS | QNGHTFPLT |
| | (SEQ ID | (SEQ ID | (SEQ ID | (SEQ ID | (SEQ ID | (SEQ ID |
| | NO: 58) | NO: 59) | NO: 60) | NO: 61) | NO: 62) | NO: 63) |

### Example 8

### Expression of 8H5 Single Chain Antibody and Detection of its Activities

The variable region genes of the heavy and light chains of 8H5 antibody gene were linked with a nucleic acid encoding a short peptide (GGGGS) to form the DNA fragment encoding a single chain antibody. 8H5 HF1/8H5 HR1 were used as the primer pair to amplify the variable region DNA fragment of 8H5 heavy chain. 8H5 KF1/8H5 KR1 were used as the primer pair to amplify the variable region DNA fragment of 8H5 light chain. The sequences of these primers are shown in Table 10. The amplified DNA fragments were recovered, and used as primers and templates for each other to carry out overlapping extension through another round of PCR amplification. A small number of full-length single chain antibody DNA fragments were obtained. Then the full-length DNA fragments were used as templates and 8H5 HF1/8H5 KR1 were used as primers to amplify a large number of the full-length DNA fragments. The amplified DNA fragments were recovered, digested with BamH I and Sal I, and cloned into prokaryotic expression vector pTO-T7. Using ER2566 E. coli as host cells, the single chain antibody proteins were expressed using standard methods. The expressed proteins were in the form of insoluble inclusion bodies. The inclusion bodies were broken up by ultrasound treatment, and the resulting sediments were purified using standard methods. The purified sediments were dissolved in 8M urea. The urea solution was dialyzed slowly in 1×PBS to allow the proteins to re-nature. The dialyzed solution was centrifuged at 12000 rpm for 10 min to remove the remaining sediments. Finally, the purified single chain antibody solution was tested for activities.

TABLE 10

Single chain antibody and chimeric antibody
cloning primers.

| Primer | |
|---|---|
| Name | Primer Sequence |
| 8H5 HF1 | 5′-TTGGATCCCAGGTTCAGCTGCAGCA-3′ |
| | (SEQ ID NO: 109) |
| 8H5 HR1 | 5′-gCTACCACCCCCTCCAgATCCgCCACCTCCTGAGGAG |
| | ACGGTGACGGTTCCTTGAC-3′ |
| | (SEQ ID NO: 110) |
| 8H5 KF1 | 5′-ATCTggAgggggTggTAgCggTggAggCgggAgTGAA |
| | ATCGTGCTCACCCA-3′ |
| | (SEQ ID NO: 111) |
| 8H5 KR1 | 5′-TTTGTCGACCCGTTTTATTTCCAGCTTGGTCCCCCCTC |
| | CGAA-3′ |
| | (SEQ ID NO: 112) |
| 8H58CHF1 | 5′-TCCTGCTACTGATTGTCCCTGCATATGTCCTGTCCCAG |
| | GTTCAGC TGCAGCAG-3′ |
| | (SEQ ID NO: 113) |
| 8H5VHR | 5′-TTTCTCGAGTGAGGAGACGGTGACTGAGGTTCC-3′ |
| | (SEQ ID NO: 114) |
| SH58CHF2 | 5′-TTTGGATCCATGGGAAGGCTTACTTCTTCATTCCTGCT |
| | ACTGATTGTCCC-3′ |
| | (SEQ ID NO: 115) |
| 8H58CKF | 5′-GCTGCTGCTGTGGCTTACAGATGCAAGATGTGAAATCG |
| | TGCTCACCC-3′ |
| | (SEQ ID NO: 116) |
| 8H5VKR | 5′-TTTCTCGAGCCGTTTTATTTCCAGCTTGGTCCCCCCTC |
| | C-3′ |
| | (SEQ ID NO: 117) |
| 8H58CKF2 | 5′-TTTGAATTCATGTCTGTGCCAACTCAGGTCCTGGGGTT |
| | GCTGCTGCTGTGGCTTAC-3′ |
| | (SEQ ID NO: 118) |
| 10F7 VHF | 5′-TTTGAATTCCAGGTCCAACTGCAGCAG-3′ |
| | (SEQ ID NO: 119) |
| 10F7 VHF | 5′-GCTACCACCCCCTCCAGATCCGCCACCTCCCGATGATA |
| | CGGTGACCG-3′ |
| | (SEQ ID NO: 120) |
| 10F7 VKF | 5′-ATCTGGAGGGGGTGGTAGCGGTGGAGGCGGGAGTGACA |
| | TCCTGATGACCCAA-3′ |
| | (SEQ ID NO: 121) |
| 10F7 VKR | 5′-TTTCTCGAGCCGTTTGATTTCCAGCTTG-3′ |
| | (SEQ ID NO: 122) |
| 10F78CHF | 5′-TCCTGCTACTGATTGTCCCTGCATATGTCCTGTCCCAG |
| | GTCCAACTGCAGCAG-3′ |
| | (SEQ ID NO: 123) |
| 10F7VHR | 5′-TTTCTCGAGCGATGATACGGTGACCGAGGTGCCTTGAC |
| | CCCAG-3′ |
| | (SEQ ID NO: 124) |
| 10F78CHF: | 5′-TTTGGATCCATGGGAAGGCTTACTTCTTCATTCCTGCT |
| | ACTGAT-3′ |
| | (SEQ ID NO: 125) |
| 10F78CKF | 5′-GCTGCTGCTGTGGCTTACAGATGCAAGATGTGACATCC |
| | TGATGACCCAATC-3′ |
| | (SEQ ID NO: 126) |
| 10F7VKR | 5′-TTTCTCGAGAGCCCGTTTTATTTCCAG-3′ |
| | (SEQ ID NO: 127) |
| 10F78CKF2 | 5′-TTTGAATTCATGTCTGTGCCAACTCAGGTCCTGGGGTT |
| | GCTGCTGCTGTGGCTTAC-3′ |
| | (SEQ ID NO: 128) |
| 4D1VHF1 | 5′-CTCTTTTTGGTATCAACAGCAACAGGTGTCCATTCCCA |
| | GGTCCAA CTGC-3′ |
| | (SEQ ID NO: 129) |
| 4D1VHR | 5′-TTTCTCGAGTGAGGAGACGGTGACCG-3′ |
| | (SEQ ID NO: 130) |
| 4D1VHF2 | 5′-TTTGGATCCATGGGATGGTCCTGTATCATTCTCTTTTT |
| | GGTATCAACAGC-3′ |
| | (SEQ ID NO: 131) |
| 4D1VKF | 5′-TTTGAATTCATGATGGTCCTTGCTCAGTTTCTTGGGTT |
| | C-3′ |
| | (SEQ ID NO: 132) |
| 4D1VKR | 5′-TTTCTCGAG AGCCCGTTTTATTTCCAG-3′ |
| | (SEQ ID NO: 133) |

Competitive ELISA method was applied to determine the activity of the purified 8H5 single chain antibody. Avian influenza polyclonal antibodies were pre-coated to the polystyrene plate and blocked with BSA, 50 μl above mentioned monoclonal antibody solution and 50 μl avian influenza H5 virus were put in the testing well. 50 μl 1×PBS and 50 μl subtype H5 avian influenza virus were put in the negative control wells, while 50 μl polyclonal antibody solution and 50 μl subtype H5 avian influenza virus were put in the positive control wells. The solution in the wells was gently mixed, incubated at 37° C. for 1 hr, then HRP labeled avian influenza polyclonal antibodies were added as secondary antibodies. The solution was incubated for another 0.5 hr and color was allowed to develop at 37° C. for 15 min after the addition of Developing Buffers A and B. The results were read with the microplate analyzer after the developing reaction had been stopped. The average value of negative controls was 1.871, the average value of positive controls was 0.089, and the average value of the testing wells was 0.597. The results showed that the initially purified 8H5 single chain antibody proteins bad high reaction activities.

26 strains of H5N1 viruses were chosen for HI assay to identify the reaction activities between the viruses and the 8H5 single chain antibody. 25 μl PBS was added to each well of a 96-wells plate. 25 μl 8H5 single chain antibody solution (0.08 mg/ml) was added to the first well and mixed thoroughly. 25 μl of the mixture from the first well was added to the second well and so on to dilute the antibody. 25 μl of each of the viruses were added to the wells separately, incubated at room temperature for 30 min. Then 50 μl of 0.5% chicken red blood cells were added to each well and incubated at room temperature for 30 min to allow blood agglutination. The results showed that 8H5 single chain antibody had HA inhibition activity to 16 of the 26 virus strains tested (Table 11).

### Example 9

### Expression of Single Chain Antibodies of 10F7 and 4D1 and Test of their Activities

As described in Example 8, the variable region genes of the heavy and light chains of each antibody were linked with a nucleic acid encoding a short peptide (GGGGS) to form the DNA fragment encoding a single chain antibody. Use 10F7 VHF/10F7 VHR as the primer pair to amplify the variable region DNA fragment of 10F7 heavy chain. Use 10F7 VKF/10F7 VKR as the primer pair to amplify the variable region DNA fragment of 10F7 light chain. Use 4D1 VHF/4D1 VHR as the primer pair to amplify the variable region DNA fragment of 4D1 heavy chain. Use 4D1 VKF/4D1 VKR as the primer pair to amplify the variable region DNA fragment of 4D1 light chain.

Use 10F7 VHF/10F7 VKR as primers to amplify the overlapping 10F7 single chain DNA fragment. Use 4D1 VHF/4D1 VKR as primers to amplify the overlapping 4D1 heavy chain DNA fragment. The amplified DNA fragments were recovered, digested with BamH I and Sal I, and cloned into prokaryotic expression vector pTO-T7 digested with the same restriction enzymes. Using ER2566 E. coli as host cells, the single chain antibody proteins were expressed using standard methods. The expressed proteins were in the form of insoluble inclusion bodies. The inclusion bodies were broken up by ultrasound treatment, and the resulting sediments were purified using standard methods. The purified sediments were dissolved in 8 M urea. The urea solution was dialyzed slowly in 1×PBS solution, centrifuged at 1200 rpm for 10 min to remove the remaining sediments. The final purified single chain antibody solution was tested for activities.

Select 26 strains of H5N1 viruses to test the activities of the above purified 10F7 and 4D1 single chain antibodies using HI assay as described above. The concentration of 10F7 single chain antibody is used at 1.06 mg/ml. The concentration of 4D1 single chain antibody is used at 0.34 mg/ml. The 4D1) single chain antibody exhibits HA inhibition activity against 23 of the virus strains. The 10F7 single chain antibody shows HA inhibition activity against 14 of the virus strains (Table 11).

TABLE 11
HA inhibition activities of the three single chain antibodies
against the 25 H5N1 viruses.

| H5N1 | ScFv | | |
|---|---|---|---|
| Virus Strains | 4D1 | 10F7 | 8H5 |
| A1 | >8 | >8 | 3.5 |
| A2 | >8 | >8 | 4.5 |
| A3 | >8 | >8 | 3.5 |
| A5 | >8 | >8 | 4 |
| A6 | 7 | 7.5 | 3 |
| A7 | 6 | 6.5 | 3 |
| A8 | >8 | 6 | 2.5 |
| B1 | >8 | 7 | 4 |
| B2 | 0 | 0 | 0 |
| B3 | >8 | >8 | 5 |
| B4 | >8 | >8 | 5 |
| B5 | 0 | 0 | 0 |
| B6 | >8 | 7 | 3.5 |
| B7 | >8 | 7 | 4 |
| B8 | >8 | 7 | 3 |
| C2 | >8 | 2 | 1 |
| C3 | >8 | 1 | 0 |
| D1 | >8 | 2.5 | 2.5 |
| D2 | 7.5 | 2.5 | 2 |
| E1 | 1 | 0 | 0 |
| E2 | 1 | 0 | 0 |
| F2 | 5 | 0 | 0 |
| F3 | 3 | 0 | 0 |
| G1 | 3.5 | 0 | 0 |
| H1 | 6 | <1 | 0 |
| H2 | 4 | 0 | 0 |

HI titer is diluted by the “n”th power of 2. “n” is the numbers shown in the table.

The activity of the above purified 10F7 single chain antibody was tested using the neutralization method. 7 virus strains that were isolated from chicken, duck and various wild birds in Hong Kong, Indonesia, Qinghai and other areas during the period from 2002 to 2006 were used to test the activity of 10F7 using the HI assay. The antibody showed good neutralization activity against 5 of the virus strains (Table 12). At 64 times of dilution, the antibody was still able to inhibit virus infection of host cells.

TABLE 12
10F7 single chain antibody neutralization test results.

| Virus strain | dilution of 10F7 scFv |
|---|---|
| CK/HK/Yu22/02 | 64 |
| DK/IDN/MS/04 | 16 |
| CK/IDN/2A/04 | 32 |
| BhGs/QH/15/05 | 16 |
| CK/HK/213/03 | <1 |
| CP Heron/HK/18/05 | 8 |
| Oriental Magpie Robin/HK/366/2006 | <1 |

### Example 10

### Expression of Chimeric Antibodies and Test of the Antibody Activities

Signal peptides were added to the genes of antibody heavy chain and light chain variable regions, and then cloned into a eukaryotic expression plasmid containing the human gamma1 heavy chain and the kappa light chain constant regions. The plasmid pcDNA3.1-AH contained the human gamma1 heavy chain constant region DNA sequence. The plasmid pcDNA3.1-Ak contained the kappa light chain constant regions.

Use 8H58CHF1/8H5VHR as primer pairs to amplify the 8H5 heavy chain variable region sequence with partial signal peptide. The amplified fragment was used as the PCR template and 8H58CHF2/8H5VHR were used as primer pairs to amplify the 8H5 heavy chain variable region sequence with the complete signal peptide. The amplified sequence was cloned into the plasmid pcDNA3.1-AH digested with Bam HI/Xho I. The resulting plasmid was the expression plasmid pcDNA3.1-AH8H5 for the human-mouse chimeric heavy chain. Use 8H58CKF1/8H5VKR1 as primer pairs to amplify the 8H5 light chain variable region sequence with partial signal peptide. The amplified fragment was used as the PCR template and 8H58CKF2/8H5VKR1 were used as primer pairs to amplify the 8H5 light chain variable region sequence with the complete signal peptide. The amplified sequence was cloned into the plasmid pcDNA3.1-Ak digested with EcoR I/Xho I. The resulting plasmid was the expression plasmid pcDNA3.1-Ak8H5 for the human-mouse chimeric light chain.

Use 10F78CHF1/10F7VHR as primer pairs to amplify the 10F7 heavy chain variable region sequence with partial signal peptide. The amplified fragment was used as the PCR template and 10F78CHF2/10F7VHR were used as primer pairs to amplify the 10F7 heavy chain variable region sequence with the complete signal peptide. The amplified sequence was cloned into the plasmid pcDNA3.1-AH digested with Bam HI/Xho I. The resulting plasmid was the expression plasmid pcDNA3.1-AH10F7 for the human-mouse chimeric heavy chain. Use 10F78CKF1/10F7VKR as primer pairs to amplify the 10F7 light chain variable region sequence with partial signal peptide. The amplified fragment was used as the PCR template and 10F78CKF2/10F7VKR were used as primer pairs to amplify the 10F7 light chain variable region sequence with the complete signal peptide. The amplified sequence was cloned into the plasmid pcDNA3.1-Ak digested with EcoR I/Xho I. The resulting plasmid was the expression plasmid pcDNA3.1-Ak10F7 for the human-mouse chimeric light chain.

Use 4D1VHF1/4D1VHR as primer pairs to amplify the 4D1 heavy chain variable region sequence with partial signal peptide. The amplified fragment was used as the PCR template and 4D1VHF2/4D1VHR were used as primer pairs to amplify the 4D1 heavy chain variable region sequence with the complete signal peptide. The amplified sequence was cloned into the plasmid pcDNA3.1-AH digested with Bam HI/Xho I. The resulting plasmid was the expression plasmid pcDNA3.1-AH4D1 for the human-mouse chimeric heavy chain. Use 4D1VKF/4D1VKR as primer pairs to amplify the 4D1 light chain variable region sequence with the signal peptide. The amplified sequence was cloned into the plasmid pcDNA3.1-Ak digested with EcoR I/Xho I. The resulting plasmid was the expression plasmid pcDNA3.1-Ak4D1 for the human-mouse chimeric light chain.

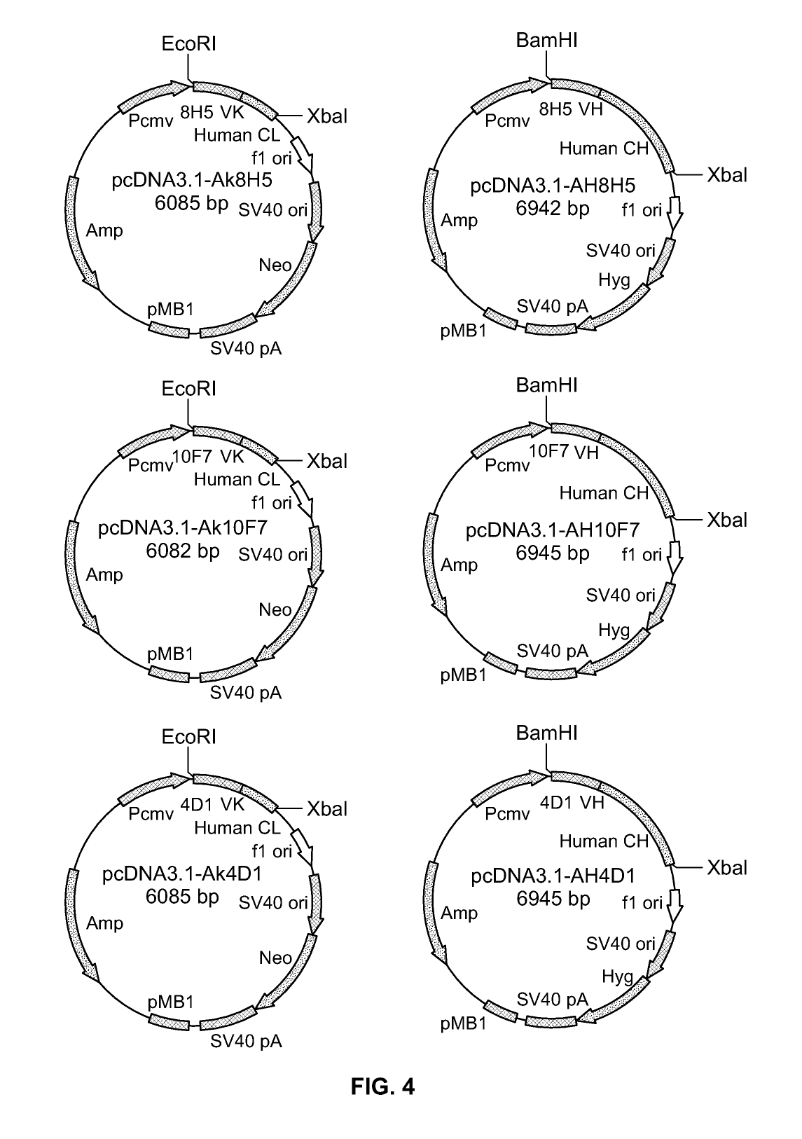

FIG. 4 shows the schematic diagrams of the structures of the expression vectors for the three chimeric antibodies.

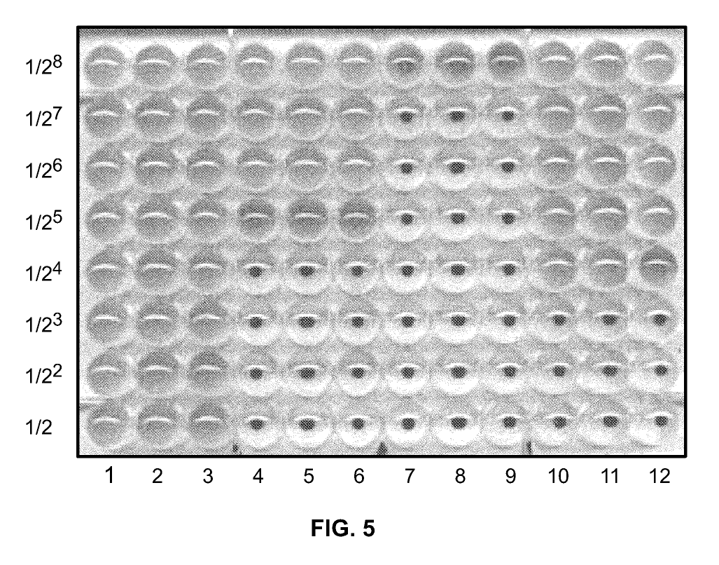

The plasmids carrying the chimeric heavy chain and the chimeric light chain were transformed into the 293 FT cells together through the calcium phosphate transformation method. The supernatant of the cell culture was collected and sedimented with saturated ammonium sulfate to obtain the initial purified chimeric antibodies (cAb). The concentration of the cAb and the mouse mAb solutions was adjusted to 0.7 μg/ml. The virus strain Ck/HK/Yu22/02 was used for the HI assay to test the antibody activities. The results showed that the activities of the three cAb were the same as their respective mouse mAb (FIG. 5).

23 H5N1 virus strains were selected to test the activities of the initial purified 10F7 and 4D1 cAb through the HI assay as described above. The results showed that both cAb had HA inhibition activities against all 23 virus strains (Table 13).

TABLE 13
The HI test results of two chimeric antibodies against
23 H5N1 avian influenza virus strains.

| Virus | | |
|---|---|---|
| No. | 4D1cAb | 10F7cAb |
| A1 | 5 | 6.5 |
| A2 | 5.5 | 7 |
| A3 | 5.5 | 6.5 |
| A4 | 6 | 7 |
| A6 | 4.5 | 5 |
| A7 | 4 | 5.5 |
| A8 | 5.5 | 5.5 |
| B1 | >8 | >8 |
| B4 | 5.5 | 6 |
| B6 | 6.5 | 6 |
| B7 | 7 | 7 |
| B8 | 4 | 4.5 |
| C1 | 7.5 | >8 |
| C2 | 6 | 6 |
| C3 | 4 | 2.5 |
| D1 | 5 | 2.5 |
| E1 | 5.5 | 6 |
| E2 | 7.5 | >8 |
| F2 | 1 | 2.5 |
| F3 | 1 | >8 |
| G1 | 1 | 6.5 |
| H1 | 1 | 5.5 |
| H2 | 1 | 8 |

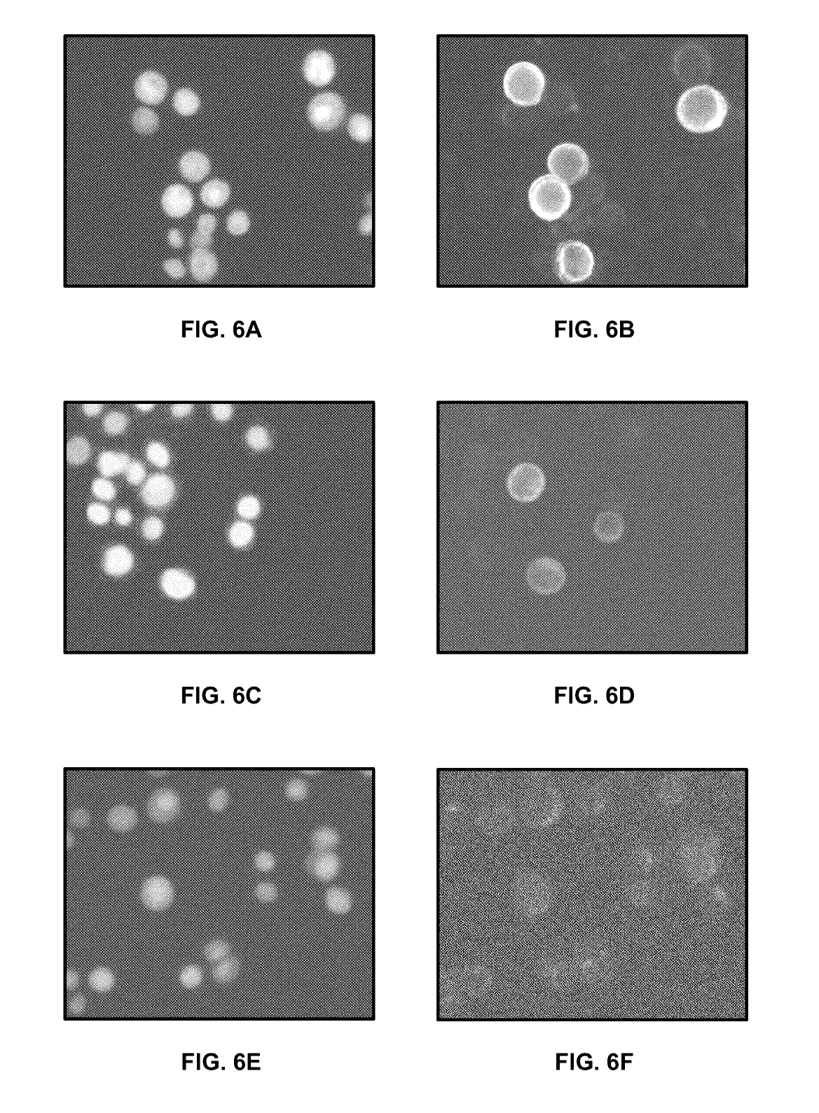

The activities of the cAb were further tested using immuno-fluorescent assays. Glass slides were put in 24-well cell culture plates. Insect SF21 cells were plated on the glass slides. Avian influenza HA proteins were expressed in the SF21 cells through the Insect cell—Baculovirus expression system. The cells expressing HA proteins were washed in PBS, fixed with 4% polyformaldehyde, blocked with goat antiserum. 4D1 or 10F7 cAb was added to the cells and incubated for 1 hour at room temperature. A specific anti-HBV cAb was used as a negative control. A fluorescent labeled goat-anti-human antibody (Sigma, St. Louis, Mo., USA) was added as the secondary antibody, and incubated for half a hour. The cell nucleus was stained with DAPI (Sigma, St. Louis, Mo., USA) for 10 minutes. The stained sample was observed under fluorescent microscope (Nikon). The results in FIG. 6 showed that the 4D1 and 10F7 cAb could specifically bind to the avian influenza virus HA proteins expressed in the SF21 cells.

### Example 11

### Screening of Short Peptides that Simulate the Antigen Sites Binding to mAb from Bacteriophage Display 7aa Peptide Library

The phage display 7aa peptide library of the New England Biolabs company was used to screen 7aa peptides that could bind to 8H5 mAb or 3C8 mAb. The screening was performed according to the manufacturer's instruction. The screening procedures were briefly describe below.

50 μl Protein A—Agarose medium (50% water suspension) was aliquoted into a microcentrifuge tube. 1 ml TBS+0.1% Tween (TBST) solution was added into the tube. The tube was gently tabbed or vibrated to re-suspend the agarose media. The tube was centrifuged at low speed for 30 sec to precipitate the agarose media. The supernatant was carefully removed. The precipitated agarose media was re-suspended in 1 ml blocking buffer and incubated at 4° C. for 60 min with occasional mixing. Meanwhile, 2×10¹¹ phage particles (the equivalent of 10 μl of the original phage library) and 300 ng of mAb were diluted with TBS buffer to the final volume of 200 μl. The final concentration of the mAb was 10 nm. The solution was incubated at room temperature for 20 min. After the blocking reaction, the media was precipitated by low speed centrifugation, and then washed with 1 ml TBS for a total of four times, each time repeating the centrifugation after the wash. The phage-mAb mixture was added to the washed media, gently mixed, and incubated at room temperature for 15 minutes with frequent mixing. The media was precipitate with low speed centrifugation. The supernatant was discarded. The media was washed with 1 ml TBTS for 10 times. Then the media was re-suspended in 1 ml 0.2M Glycine-HCl (pH 2.2) and 1 mg/ml BSA, incubated at room temperature for 10 min to release the bound phage particles. The mixture was centrifuged for 1 min and the supernatant was carefully removed to another microcentrifuge tube. The supernatant was immediately neutralized with 150 μl 1M Tris-HCl, pH 9.1. Approximately 1 μl of the foregoing solution was used to check the titer of the phage. The remaining solution was added into 20 ml ER2738 host cells that were at early log phase growth. The host cells were cultured with vibration at 37° C. for 4.5 hour. The cell culture was transferred to a 50 ml centrifuge tube and centrifuged at 10,000 rpm for 20 min. The top 80% of the supernatant was collected and one-sixth volume of PEG/NaCl solution (20% PEG-8000, 2.5M NaCl) was added. The solution was set at 4° C. for 1 hour, and then centrifuged at 10,000 rpm at 4° C. for 15 min. The supernatant was discarded and the precipitated phage was suspended in 200 μl PBS and stored at 4° C. The above-mentioned procedures were repeated for another screening.

The overnight cultured ER2738 host cells were diluted into LB media at the ratio of 1:10 and aliquoted into culture tubes (1 ml/tube). For each mAb screening, 10 blue single colony phage plaques on LB/IPTG/Xgal culture plates that had undergone three rounds of screening were selected and inoculated into the foregoing culture tubes. The cell cultures were incubated with vibration at 37° C. for 4.5 hr. The cell cultures were transferred to 1.5 ml centrifuge tubes and centrifuged at 10,000 g for 10 min. 200 μL of supernatant was collected. Phage ssDNA was isolated from the supernatant using small quantity M13 Isolation and Purification Reagent Kit (Shanghai Huashun Bioengineering Co., Ltd.) following the manufacturer's instruction. The sequences of the inserted 7aa peptides were obtained by Shanghai Boya Biotechnology Co., Ltd. and shown in Table 14.

TABLE 14

The Amino Acid Sequences of the 7aa peptides
that bind to 8H5 mAb or 3C8 mAb.
(The nucleic acid sequences in SEQ ID Nos.
13, 14 and 15 encode peptides of SEQ ID Nos.
64, 68, and 70 respectively.)

| Monoclonal | 7-aa peptide | |
|---|---|---|
| Antibody | sequences | Sequence No. |
| 8H5 | HGMLPVY | SEQ ID No: 64 |
| | PPSNYGR | SEQ ID No: 65 |
| | PPSNFGK | SEQ ID No: 66 |
| | GDPWFTS | SEQ ID No: 67 |
| | NSGPWLT | SEQ ID No: 68 |
| 3C8 | WPPLSKK | SEQ ID No: 70 |
| | NTFRTPI | SEQ ID No: 71 |
| | NTFRDPN | SEQ ID No: 72 |
| | NPIWTKL | SEQ ID No: 73 |

### Example 12

### Detection of 7aa Peptides Activities

The three bacteriophages containing the 7aa peptides of 8H5A, 8H5E and 3C8A were amplified in large numbers. They were dissolved in PBS after being precipitated with PEG. Phage titer was between 10¹¹ and 10¹². Microplates were pre-coated with monoclonal antibodies 8H5, 4A1, 9N7 and 4D11 at 5 μg/ml. The plates were blocked with PBS containing 5% milk. The three bacteriophages were serially diluted; and added to the plates. The reaction was carried on for 1 hr. Then the plates were washed for 5 times. 1:5,000 diluted mouse anti-M13/HRP antibody (Amersham Phamarcia Biotech, UK) was added as the secondary antibody and incubated for 0.5 hr. The results were read after the reaction was completed. The results are shown in Table 15, which demonstrated that the specific reactions between the peptide 8H5A and the monoclonal antibody 8H5 were good, and the specific reactions between 8H5A and the other three monoclonal antibodies were weak. The specific reaction between 8H5E and monoclonal antibody 8H5 was relatively poor.

TABLE 15
Detection results of the specific binding activity of 7aa Peptides to
monoclonal antibodies

7aa Peptide in Bacteriophage

| Monoclonal Antibody | 8H5A (1:1000) | 8H5E (1:1000) |
|---|---|---|
| 8H5 | 0.559 | 0.25 |
| 4A1 | 0.158 | 0.142 |
| 9N7 | 0.062 | 0.065 |
| 4D11 | 0.118 | 0.078 |

### Example 13

### Screening of Short Peptides that Simulate the Antigen Site Binding to 8H5 mAb from Phage Display 12aa Peptide Library

The phage display 12aa peptide library of the New England Biolabs company was used to screen 12aa peptides that could bind to 8H5 mAb. The screening was performed according to the manufacturer's instruction. The detailed experimental procedures were the same as in Example 11.

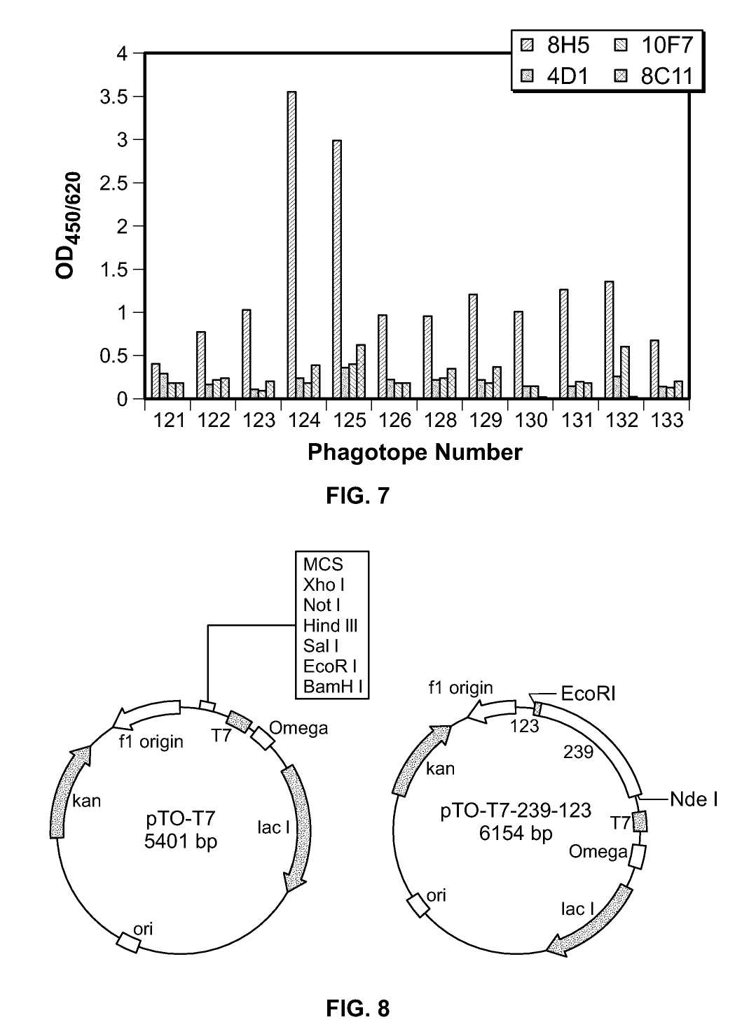

After the third round of screening, approximately 1 μl of the phage solution was used to determine the phage's titer. Single colony phage plaques were selected and inoculated into ER2738 bacteria at log phase growth. The inoculated bacteria cultures were incubated at 37° C. for 4.5˜5 hr. Then they were centrifuged to collect the supernatant for ELISA test. Mouse 8H5 mAb was imbedded on the ELISA microplates at the concentration of 10 μg/ml. The phage solution was used as the primary antibody. 1:5,000 diluted anti-M13/HRP antibody (Amersham Pharmarcia Biotech, UK) was used as the secondary antibody. The avian influenza antibodies 4D1 mAb, 10F7 mAb and the anti-HEV E2 8C11 mAb were used as negative controls for the mouse mAb. FIG. 7 shows the test results of 12 phage peptides that exhibited better binding activities. The tests demonstrated that most of the phage peptides had OD values against the target 8H5 mAb that were three times higher than the controls, indicating that the peptides had good specificity.

Phage DNA was isolated using the phage ssDNA isolation reagent kit (Omega, USA) following the manufacturer's instruction. The isolated DNA was sequenced. The nucleic acid and amino acid sequences of the twelve 12aa peptides were obtained (Table 16).

TABLE 16

The sequences of the 12aa peptides that bind
to 8H5 mAb.

| Peptide | Amino Acid | |
|---|---|---|
| section No. | Sequence | Base Sequence |
| 121 | MEPVKKYPTRSP | ATGGAGCCGGTGAAG |
| | (SEQ ID NO: 74) | AAGTATCCGACGCGT |
| | | TCTCCT |
| | | (SEQ ID NO: 75) |
| 122 | ETQLTTAGLRLL | GAGACTCAGCTGACT |
| | (SEQ ID NO: 76) | ACGGCGGGTCTTCGG |
| | | CTGCTT |
| | | (SEQ ID NO: 77) |
| 123 | ETPLTETALKWH | GAGACGCCTCTTACG |
| | (SEQ ID NO: 78) | GAGACGGCTTTGAAG |
| | | TGGCAT |
| | | (SEQ ID NO: 79) |
| 124 | QTPLTMAALELF | CAGACGCCGCTGACT |
| | (SEQ ID NO: 80) | ATGGCTGCTCTTGAG |
| | | CTTTTT |
| | | (SEQ ID NO: 81) |
| 125 | DTPLTTAALRLV | GATACTCCGCTGACG |
| | (SEQ ID NO: 82) | ACGGCGGCTCTTCGG |
| | | CTGGTT |
| | | (SEQ ID NO: 83) |
| 126 | TPLTLWALSGLR | ACGCCGCTTACGCTT |
| | (SEQ ID NO: 84) | TGGGCTCTTTCTGGG |
| | | CTGAGG |
| | | (SEQ ID NO: 85) |
| 128 | QTPLTETALKWH | CAGACGCCTCTTACG |
| | (SEQ ID NO: 86) | GAGACGGCTTTGAAG |
| | | TGGCAT |
| | | (SEQ ID NO: 87) |
| 129 | QTPLTMAALELL | CAGACGCCTCTGACT |
| | (SEQ ID NO: 88) | ATGGCGGCTCTTGAG |
| | | CTTCTT |
| | | (SEQ ID NO: 89) |
| 130 | HLQDGSPPSSPH | CAGACGCCTCTGACT |
| | (SEQ ID NO: 90) | ATGGCGGCTCTTGAG |
| | | CTTCTT |
| | | (SEQ ID NO: 91) |
| 131 | GHVTTLSLLSLR | GGGCATGTGACGACT |
| | (SEQ ID NO: 92) | CTTTCTCTTCTGTCG |
| | | CTGCGG |
| | | (SEQ ID NO: 93) |
| 132 | FPNFDWPLSPWT | TTTCCGAATTTTGAT |
| | (SEQ ID NO: 94) | TGGCCTCTGTCTCCG |
| | | TGGACG |
| | | (SEQ ID NO: 95) |
| 133 | ETPLTEPAFKRH | GAGACGCCTCTTACG |
| | (SEQ ID NO: 96) | GAGCCGGCTTTTAAG |
| | | CGGCAT |
| | | (SEQ ID NO: 97) |

### Example 14

### Expression of Fusion Proteins Containing the 12aa Peptides 123 or 125 and Peptide 239 and Detection of their Activities

Construction of the Expression Vectors for Fusion Proteins 239-123 and 239-125

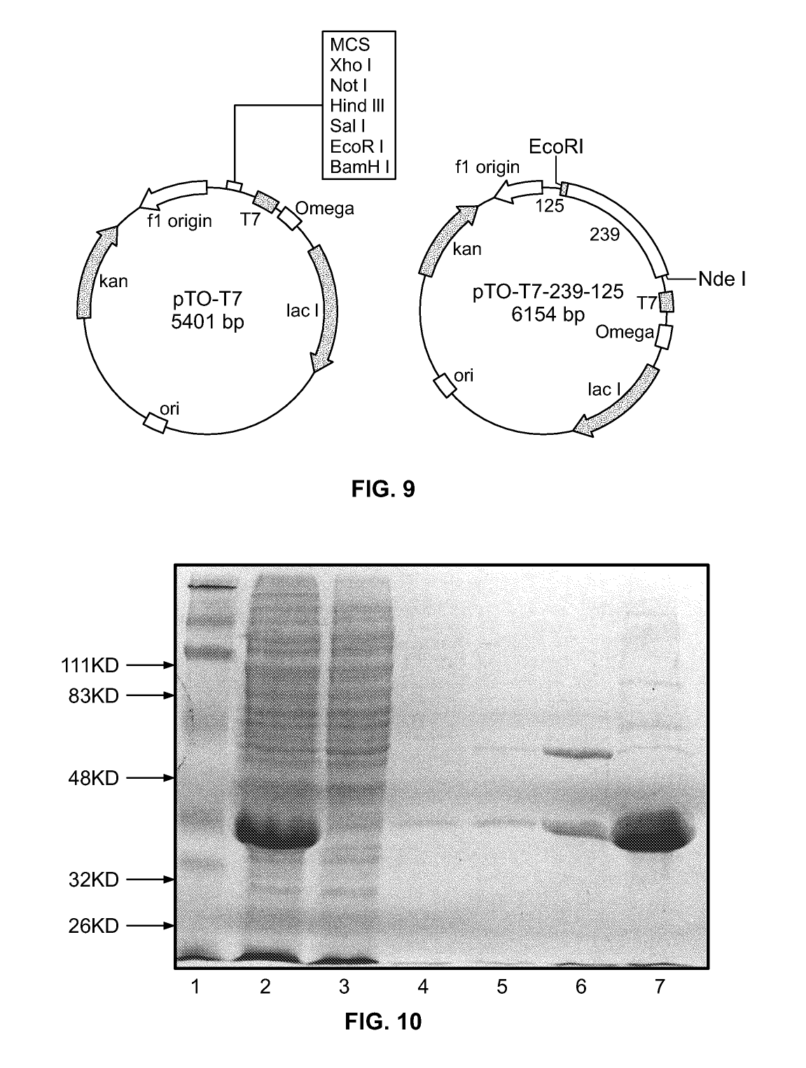

The 12aa peptides 123 or 125 were linked to the c-terminal of the peptide 239 (which was the 239 amino acid fragment from residues 368-606 of HEV ORF2) to construct prokaryotice expression vectors pTO-T7-239-123 (FIG. 8) and pTO-T7-239-125 (FIG. 9) through PCR. First, primers for the 239 gene and 12aa peptide gene were prepared. Then the 239 gene was used as the template, and the primers 239-123F/239-123R1 and 239-125F/239-125R1 were used, respectively, for the first round of PCR amplification. The PCR products were collected and purified and then used as the templates for the second round of PCR amplification. In the second round of PCR amplification, the primers 239-123F/239-123R2 and 239-125F/239-125R2 were used for the construction of the fragments 239-123 and 239-125, respectively. The generated fragments 239-123 and 239-125 were collected, digested with restriction enzymes NdeI and EcoRI, and cloned into vector pTO-T7. The vectors were transformed into E. coli ER2566, replicated and examined by restriction enzyme digestion. The positive host cell clones contained the recombinant prokaryotic expression vectors pTO-T7-239-123 and pTO-T7-239-125.

TABLE 17

Sequences of the primers for the constructs
239-123 and 239-125.

| | |
|---|---|
| Primer | Primer Sequence |

| | |
|---|---|
| 239-123F | 5′-TTT TTA CAT ATG ATA GCG CTT ACC |
| | CTG-3′ |
| | (SEQ ID NO: 134) |
| 239-123R1 | 5′-GCTACCACCACCACCAGAACCACCACCACCGCGCGGA |
| | GGGGGGGCTAAAC-3′ |
| | (SEQ ID NO: 135) |
| 239-123R2 | 5′-TA GAA TTC ATG CCA CTT CAA AGC CGT |
| | CTC CGT AAG AGG CGT CTC GCT ACC TCC ACC |
| | ACC-3′ |
| | (SEQ ID NO: 136) |
| 239-125F | 5′-TTT TTA CAT ATG ATA GCG CTT ACC |
| | CTG-3′ |
| | (SEQ ID NO: 137) |
| 239-125R1 | 5′-GCT ACC ACC ACC ACC AGA ACC ACC ACC |
| | ACC GCG CGG AGG GGG GGC TAA AAC-3′ |
| | (SEQ ID NO: 138) |
| 239-125R2 | 5′-TA GAA TTC AAC CAG CCG AAG AGC CGC |
| | CGT CGTCAG CGG AGT ATC GCT ACC TCC ACC |
| | ACC-3′ |
| | (SEQ ID NO: 139) |

Expression and Purification of the Fusion Proteins 239-123 and 239-125.

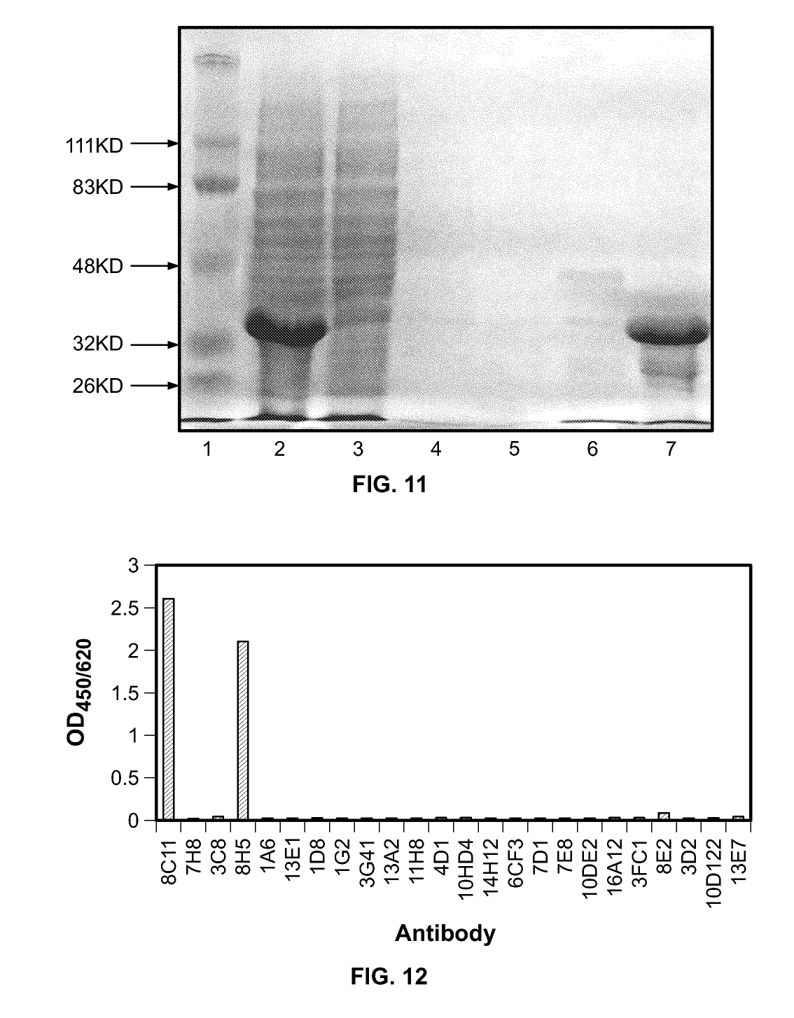

ER2566 single colonies containing vectors pTO-T7-239-123 and pTO-T7-239-125 were each inoculated into 2 ml Kn-resistant LB media. The bacteria cultures were incubated with vibration at 37° C. until the OD600 value reached about 0.5. Then the cultures were transferred at the ratio of 1:1000 to 500 ml LB media, and incubated until the OD600 value reached approximately 1.0. Then 500 μl IPTG was added into the bacteria cultures to induce protein expression at 37° C. for 4 hr. The bacteria were collected by centrifugation at 8,000 rpm for 10 min at 4° C. The supernatant was discarded. The bacteria debri was re-suspended in 20 ml lysis buffer, incubated on ice, treated with ultrasound sonication to break the bacteria. The conditions for the ultrasound treatment were as follows: working time: 10 min; treatment pulse: treating with pulse for 2 sec and stopping for 5 sec; power output: 70%. After the ultrasound treatment, the bacteria solution was centrifuged at 12,000 rpm for 10 min. The supernatant was saved (to be loaded to SDS-PAGE, Lane 3 of FIGS. 10 and 11) and the debri was re-suspended in 20 ml 2% Triton, vibrated for 30 min, and then centrifuged at 12,000 rpm for 10 min. The Triton wash was repeated once. Then the supernatant was saved (to be loaded to SDS-PAGE, Lane 4 of FIGS. 10 and 11) and the debri was re-suspended in 20 ml 2M Urea Buffer, vibrated for 30 min, and centrifuged at 12,000 rpm, for 10 min. Again, the supernatant was saved (to be loaded to SDS-PAGE, Lane 5 of FIGS. 10 and 11) and the debri was re-suspended in 20 ml 4M Urea, vibrated for 30 min, and then centrifuged at 12,000 rpm for 10 min. Furthermore, the supernatant was saved (to be loaded to SDS-PAGE, Lane 6 of FIGS. 10 and 11) and the debri was re-suspended in 20 ml 8M Urea, vibrated for 30 min, and then centrifuged at 12,000 rpm for 10 min. The supernatant was saved (to be loaded to SDS-PAGE, Lane 7 of FIGS. 10 and 11). SDS-PAGE loading samples were prepared from all of the foregoing supernatants for SDS-PAGE analysis (FIG. 10 and FIG. 11). The SDS-PAGE results showed that the proteins mostly dissolved in the 8M Urea, with a purity of 90%. The 8M Urea protein solution was dialyzed into PBS with gradient dialysis (8M Urea-4M Urea-2M Urea-PBS).

Detection of the Activities of Fusion Proteins 239-123 and 239-125

Direct ELISA Test

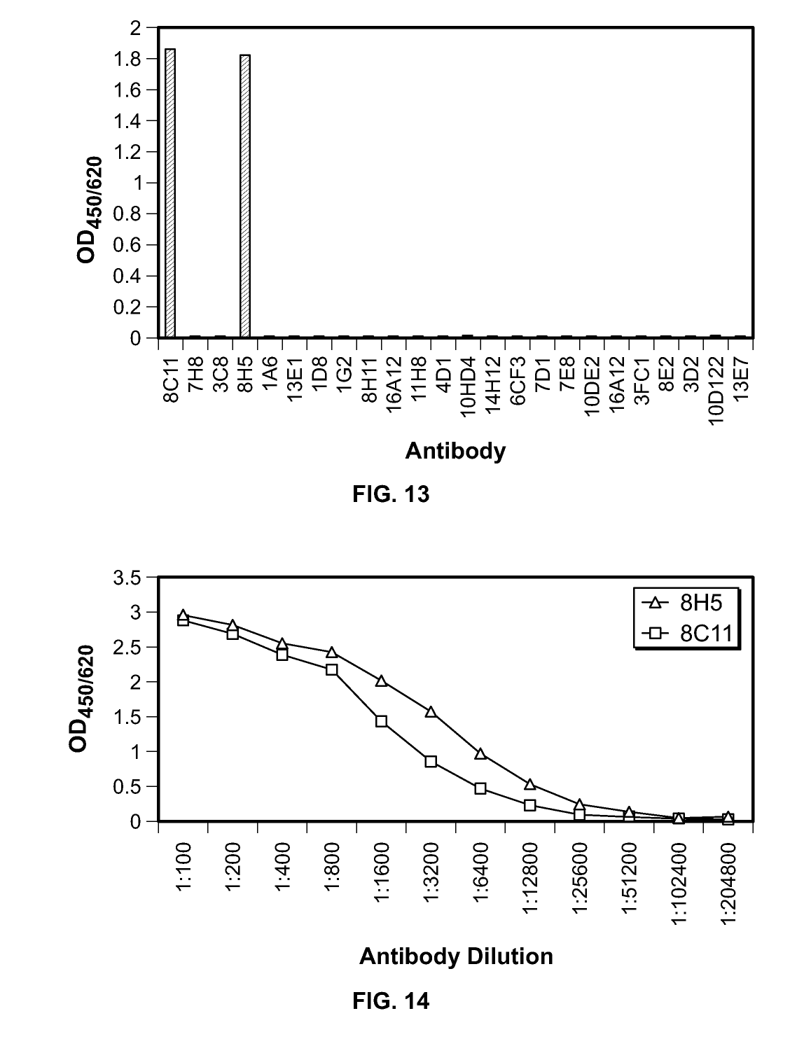

The initial purified fusion proteins 239-123 and 239-125 were separately imbedded onto 96-well plates at the concentration of 10 μg/ml at 37° C. for 2 hr. After that, the plates were washed once, treated with ED blocking buffer at 37° C. for 2 hr and then at 4° C. overnight to block non-specific binding sites. Next, the blocking buffer was discarded. Thereafter, different mouse mAb were added to the plates at 100 μl per well. There were 24 mAb, including 8C1, 7H8,3C8, 8H5, 1A6, 13E1, 1D8, 1G2,3G41, 13A2, 11H8, 4D1, 10HD4,14H12, 6CF3, 7D1, 7E8, 10DE2, 16A12, 3FC1, 8E2, 3D2, 10D122, 13E7. The 8C11 mAb was a specific anti-239 protein antibody. The 8H5 mAb was used to screen the 12aa peptides. The other 22 mAb were antibodies against the HA protein of avian influenza virus. The mAb were incubated in the plates at 37° C. for 1 hr. The plates were washed with PBST for 5 times. 100 μl GAM-HRP (1:10,000 dilution) was added to each well and incubated at 37° C. for 30 min. The plates were washed with PBST 5 times. Coloring solution was added to the plates for 10 min for color development, and then stopping buffer was added to stop the color reaction. The intensity of the color was read with a microplate reader. FIGS. 12 and 13 showed that fusion proteins 239-123 and 239-125 reacted only with 8C11 and 8H5, respectively, and did not react with any other mAb. The results indicated that fusion proteins 239-123 and 239-125 had very good antibody specificity.

### Example 15

### Expression of Fusion Proteins of 12aa Peptide No. 123 and No. 125 with HBV cAg

Construction of the Fusion Protein Expression Vectors

The aa1-149 fragment of HBV cAg expressed in E. coli could assemble into virus like particles. The gene for the aa1-149 fragment was inserted into the E. coli expression vector pTO-T7. Then the two amino acids at positions 79 and 80 of the fragment were replaced with substituent amino acids whose nucleic acid sequence contains restriction enzyme recognition sites to make the mutant HBV cAg expression plasmid pC149-mut. HBV cAg is substantially immunogenic. A foreign peptide fused to the internal MIR (major immunodominant region, aa 78-83) of HBV cAg will not change HBV cAg's ability to assemble into particles, however, the peptide epitope will be exposed from the particle surface.

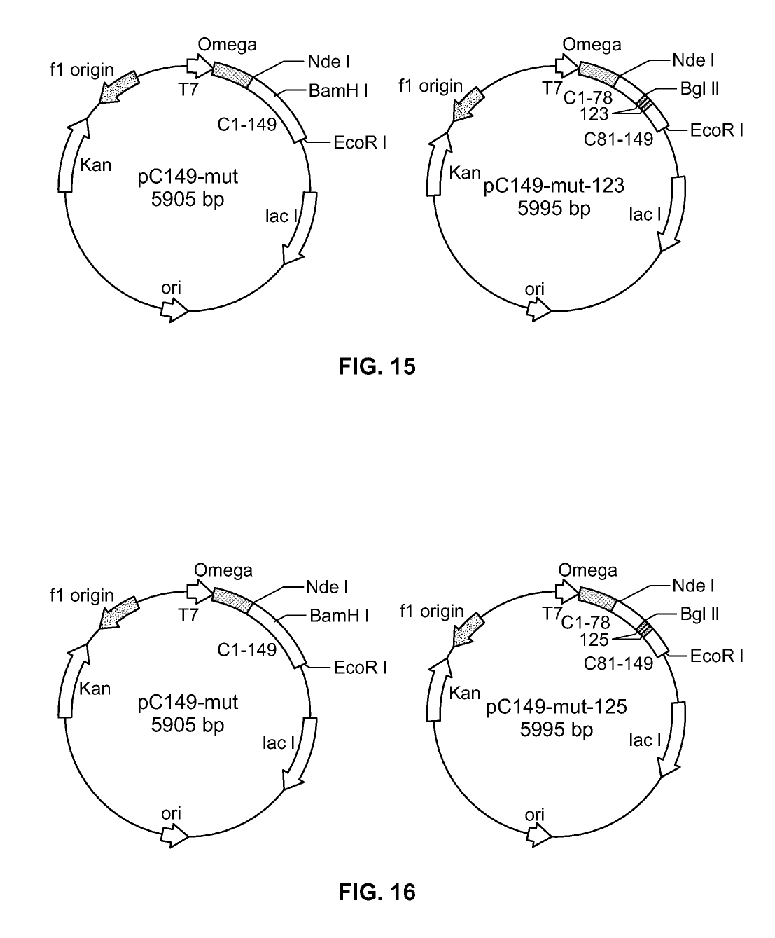

1 to 5 copies of peptides 123 and 125 were respectively inserted into the amino acid positions 79 and 80 of HBcAg to obtain a series of fusion proteins, which were called HBc-123 and HBc-125, respectively. Based on the sequences of the 12aa peptides and the vector pC149-mut, 5′-end primers containing the sequences of the 12aa peptides and 3′-end primer 149MRP were designed (Table 18, the underlined parts were the inserted peptide sequences). The plasmid pC149-mut was used as the template and the primers HBc123F2/HBcR and HBc123F2/HBcR were used for the first round of PCR amplification. The PCR products were recovered, purified and used as the template, and the primers HBc123F1/HBcR and HBc123F1/HBcR were used for the second round of PCR amplification. As a result, C149aa81-149 linking to the 12aa peptide sequence was generated. The fragment was digested with Bgl II and EcoR I and purified. The plasmid pC149-mut was digested with BamH I and EcoR I, purified, and linked with the C149 fragment containing 12aa peptide sequences. The linked products were transformed into E. coli ER2566 for expression and restriction enzyme digestion analysis. The analysis selected plasmids that had a single copy of the 12aa peptide genes inserted, which were referred to as pC149-mut-123 and pC149-mut-125, respectively. The plasmids were digested with BamH I and EcoR I. The fragments containing the 12aa peptides were digested with Bgl II and EcoR I and then linked with the digested plasmids. The recombinant prokaryotic expression plasmids containing 2 copies of the 12aa peptides were selected and called pC149-mut-D123 and pC149-mut-D125, respectively. Similarly, recombinant prokaryotic expression vectors containing 3, 4 and 5 copies of the 12aa peptides were constructed, including plasmids pC149-mut-T123, pC149-mut-F123, pC149-mut-Q123 and pC149-mut-T125, pC149-mut-F125, pC149-mut-Q125. The structures of the recombinant plasmids are shown in FIGS. 15 and 16 (only plasmids pC149-mut-123 and pC149-mut-125 were shown as examples).

TABLE 18

The sequences of the primers for the
construction of the vectors for the fusion
proteins of the 12aa peptides 123 and 125
with HBVcAg.

| | |
|---|---|
| Peptide | Primer sequences |

| | |
|---|---|
| HBc123F1 | 5′-TTT AGA TCT GGA GGA GGT GGT GAG ACG CCT |
| | CTT ACG GAG ACG GCT TTG AA TGG C-3′ |
| | (SEQ ID NO: 140) |
| HBc123F2 | 5′-CG GCT TTG AAG TGG CATGGA TCC GGT GGC |
| | GGA TCT CTG CAG GGT GGT GGA GGT TCA GG-3′ |
| | (SEQ ID NO: 141) |
| HBc125F1 | 5′-TTT AGA TCT GGA GGA GGT GGT TCT GAT ACT |
| | CCC CTG ACG ACG GCG GCT CTT CGG CTG G-3′ |
| | (SEQ ID NO: 142) |
| HBc125F2 | 5′-CG GCT CTT CGG CTG GTT GGA TCC GGT GGC |
| | GGA TCT CTG CAG GGT GGT GGA GGT TCA GG-3′ |
| | (SEQ ID NO: 143) |
| HBcR | 5′-TT GAA TTC TTA AAC AAC AGT AGT TT-3′ |
| | (SEQ ID NO: 144) |

Note:
the underlined are sequences of 12 peptides.

Expression and Purification of the Fusion Proteins

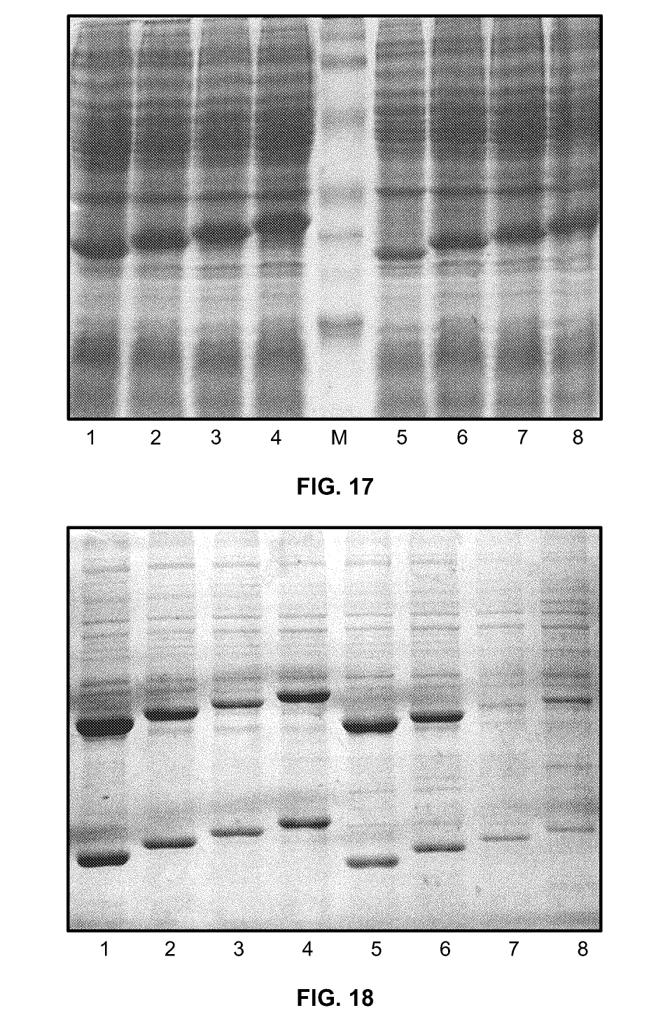

The fusion proteins expressed by the plasmids pC149-mut-D123, pC149-mut-T123, pC149-mut-F123, pC149-mut-Q123, pC149-mut-D125, pC149-mut-T125, pC149-mut-F125, pC149-mut-Q125 were called D123, T123, F123, Q123, D125, T125, F125, Q125, respectively. ER2566 bacteria containing these 8 plasmids, respectively, were each inoculated into 2 ml Kn-resistant LB media, and shaken at 37° C. until the OD600 values reached 0.5. Then the cultures were transferred at the ratio of 1:1000 to 500 ml LB media, and incubated until the OD600 value reached approximately 0.8. After that, 500 μl IPTG was added into the bacteria cultures to induce protein expression at 18° C. for 20 hr. The bacteria were collected by centrifugation at 8,000 rpm for 10 min at 4° C. The supernatant was discarded. The bacteria debri was re-suspended in 20 ml lysis buffer, incubated on ice, treated with ultrasound sonication to break the bacteria. The conditions for the ultrasound treatment were as follows: working time: 10 min; treatment pulse: treating with pulse for 2 sec and stopping for 5 sec; power output: 70%. After the ultrasound treatment, the bacteria solution was centrifuged at 12,000 rpm for 10 min. The supernatant was saved and ran on SDS-PAGE The results showed that all fusion proteins were in the supernatants (FIG. 17).

The above supernatants still contained many contaminants and needed further purification. These proteins could self-assemble into particles under suitable conditions. The self-assembly conditions of these proteins were also considered during further purification of these proteins. The following procedures were used to further purify the proteins and stimulate the proteins to self-assemble into particles: saturated ammonium sulfate was added to a final concentration of 20% of the total volume; the mixtures was then incubated on ice for 30 min; the mixture was centrifuged at 12,000 rpm for 10 min; the supernatant was discarded; the debri was re-suspended in CB Buffer containing 5% β-mercaptoethanol; shaken at 37° C. for 30 min, centrifuged at 12,000 rpm for 10 min. The supernatant was collected and dialyzed in PB5.8 Buffer (including 300 mM NaCl and 50 mMEDTA). The buffer was changed every 8 hr. After the buffer was changed 6 times, the dialyzed solution was collected and centrifuged at 12,000 rpm for 10 min. The supernatant was collected and the purity of the isolated protein was checked on SDS-PAGE. Using this method, the proteins were purified first by 20% saturated ammonium sulfate sedimentation, then CB Buffer containing 5% β-mercaptoethanol was used to stimulate the proteins to assemble into dimmers, then the protein particles were formed under the condition of low pH and high salt. Furthermore, the proteins could be further purified under these conditions (FIG. 18).

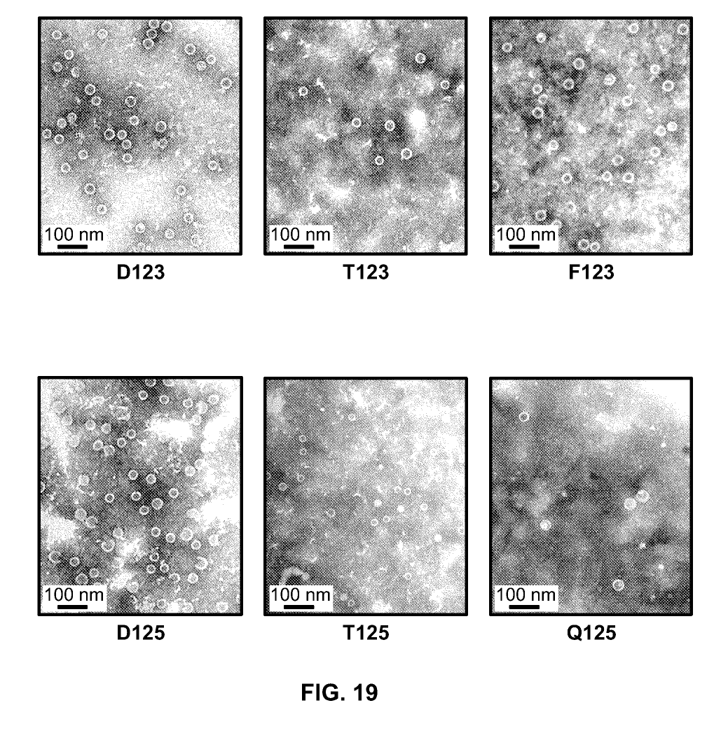

The 8 fusion proteins purified by the above method were negatively stained with 2% phosphotungstic acid and observed directly under electron microscope (FIG. 19). The assembled particles were shown as uniform hallow spheres (some particles had filled insides). The particles were in two sizes, one had a diameter of 35 nm and the other 20 nm.

### Example 16

### The Activities of the HBc-123 and HBc-125 Fusion Proteins were Tested by ELISA

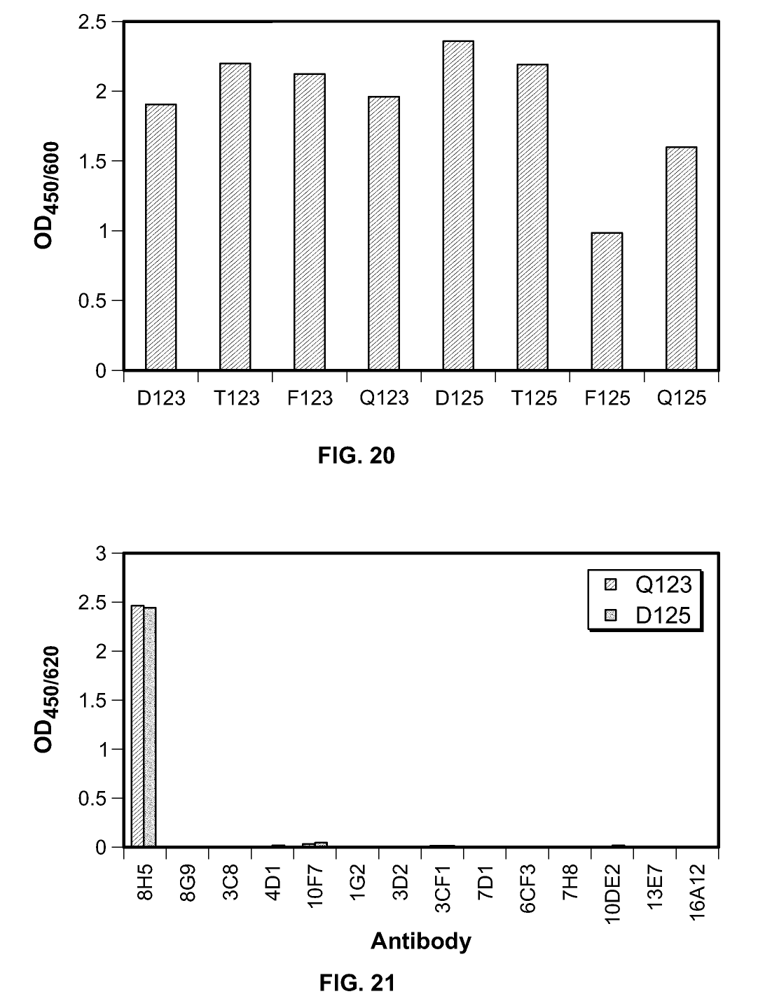

The 8 fusion proteins were separately imbedded onto 96-well plates at the concentration of 10 μg/ml at 37° C. for 2 hr. After that, the plates were washed once, treated with ED blocking buffer at 37° C. for 2 hr and then at 4° C. overnight to block non-specific binding sites. Thereafter, 100 μl of 8H5 mAb was added to each well. The mAb was incubated in the plates at 37° C. for 1 hr. The plates were washed with PBST for 5 times. 100 μl GAM-HRP (1:10,000 dilution) was added to each well and incubated at 37° C. for 30 min. The plates were washed with PBST for 5 times. Coloring solution was added to the plates for 10 min for color development. Then stopping buffer was added to stop the color reaction. The intensity of the color was read with microplate reader. FIG. 20 showed that all 8 fusion proteins bound specifically to 8H5 mAb.

Q123 and D125 proteins were tested for antibody binding specificity. The two proteins were separately imbedded onto 96-well plates at the concentration of 10 μg/ml at 37° C. for 2 hr. The plates were washed once, and treated with ED blocking buffer at 37° C. for 2 hr and then at 4° C. overnight to block non-specific binding sites. Next, different mAb were added to the plates at 100 μl per well, including a total of 14 mAb: 8H5, 8G9, 3C8, 4D1, 10F7, 1G2, 3D2, 3CF1, 7D1, 6CF3, 7H8, 10DE2, 13E7, 16A12. The 8H5 mAb was used to screen the 12aa peptides. The other 13 mAb were antibodies against the HA protein of avian influenza virus. The mAb were incubated in the plates at 37° C. for 1 hr. The plates were washed with PBST for 5 times. 100 μl GAM-HRP (1:10,000 dilution) was added to each well and incubated at 37° C. for 30 min. The plates were washed with PBST for 5 times. Coloring solution was added to the plates for 10 min for color development. Then stopping buffer was added to stop the color reaction. The intensity of the color was read with microplate reader. The results of ELISA (FIG. 21) showed that fusion proteins Q123 and D125 reacted only with 8H5, and did not react with any other mAb. The results indicated that fusion proteins Q123 and D125 had very good antibody specificity.

### Example 17

### Analysis of the Immunogenicity of Fusion Proteins HBc-123 and HBc-125

The above 8 HBc-123 and HBc-125 fusion proteins were separately mixed with equal volume of Freund's adjuvant (complete Freund's adjuvant was used for the initial immunization and incomplete Freund's adjuvant was used for booster immunization. The immunizing protein and adjuvant were injected in BALB/c mice by multiple subcutaneous injections at the dosage of 100 μg protein per mouse. 3 to 4 mice were included in a group. After the initial immunization, a booster immunization was done every other week meanwhile blood was collected from the mice eyes. The generated anti-serum were separated as follows: the blood was kept at 37° C. for 2 hr and then stored at 4° C. for overnight to allow the blood cells to agglutinate. Next, the blood was centrifuged at 4,000 g for 10 min. The supernatant which contained the anti-serum was taken and stored at −4° C. for future use.

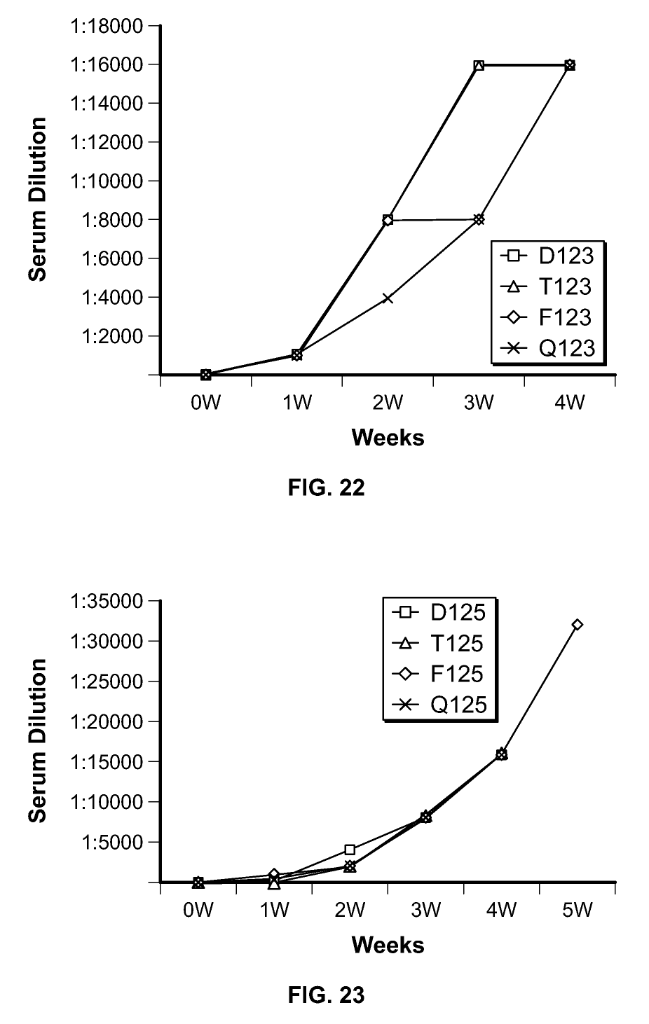

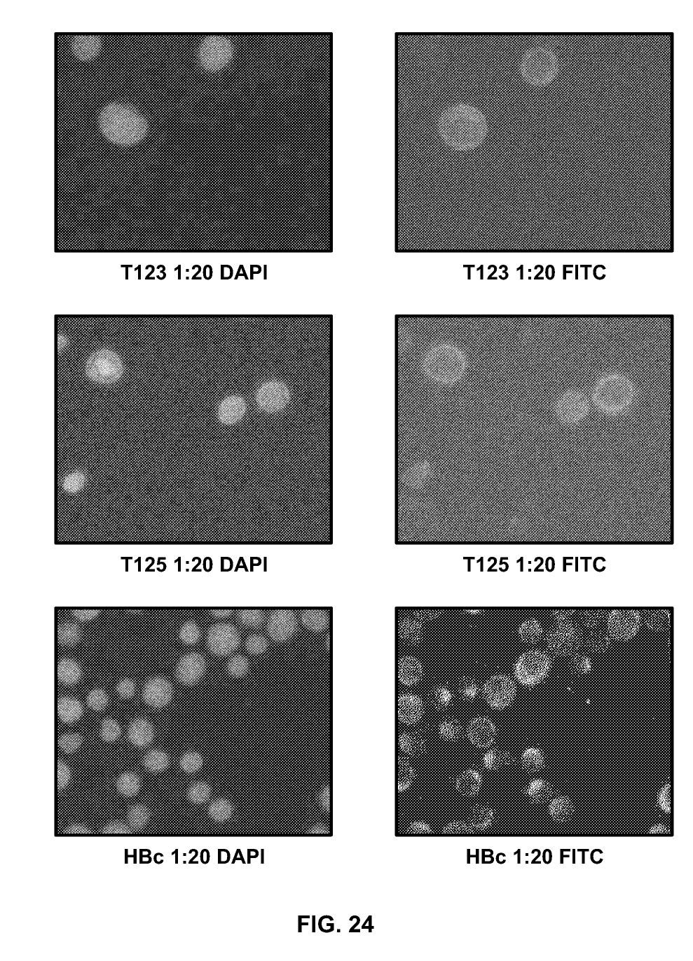

Fusion proteins 239-123 and 239-125 were imbedded on microplates at 1 μg per well. HRP-labeled goat-anti-mouse IgG was used as the secondary antibody. Accordingly, indirect ELISA could be conducted to detect specific antibodies against the 12aa peptides 123 and 125. The indirect ELISA could be used to detect anti-serum that could bind to the 12aa peptides 123 and 125, the binding specificity, the antibody titer and other related functions. Indirect ELISA test was conducted using 1:1,000 diluted anti-serum against the various HBc-123 fusion proteins and the results are shown in FIG. 22. Indirect ELISA test was conducted using 1:2,000 diluted anti-serum against the various HBc-125 fusion proteins and the results are shown in FIG. 23.

### Example 18

### Immuno-Fluorescent Detection of Mouse Anti-Serum

Glass slides were put in 24-well cell culture plates. Insect SF21 cells were plated on the glass slides. Avian influenza HA proteins were expressed in the SF21 cells through the Insect cell—Baculovirus expression system. The cells expressing HA proteins were washed in PBS, fixed with 4% polyformaldehyde, blocked with goat antiserum. 1:20 diluted mouse anti-serum against T123 and F125 were separately added to the cells and incubated for 1 hour at room temperature. Anti-HBc mouse anti-serum was used as a negative control. A fluorescent labeled goat-anti-mouse antibody (Sigma, St. Louis, Mo., USA) was added as the secondary antibody, and incubated for half an hour. The cell nucleus was stained with DAPI (Sigma, St. Louis, Mo., USA) for 10 minutes. The stained sample was observed under fluorescent microscope (Nikon). The results in FIG. 24 showed that the mouse anti-serum against T123 and F125 could specifically bind to the avian influenza virus HA proteins expressed in the SF21 cells, further confirming that the 12aa peptides 123 and 125 appropriately simulated the HA antigen sites.

### Example 19

### Expression of the Fusion Proteins of 12aa Peptides 122, 124, 128 and 129 with HBV cAg and Detection of their Activities

2 copies of the 4 12aa peptides 122, 124, 128 and 129 were inserted into the HBV cAg protein and obtained the fusion proteins HBc-122, HBc-124, HBc-128 and HBc-129, respectively.

The method for constructing the expression vectors for the fusion proteins HBc-122, HBc-124, HBc-128 and HBc-129 were the same as the construction method for the fusion proteins HBc-123 and HBc-125. The primers used in the construction method are shown in Table 19. The upstream primer for the first PCR amplification was F3, for the second PCR amplification was F2, and for the third PCR amplification was F1. The down stream primer was HBcR. Through three rounds of PCR amplification, the target 12aa peptides were linked with the C149-mut fragment. The linked fragments were digested with Bgl II and EcoR I, and inserted into the vector pC 149-mut that were digested with BamH I and EcoR I to form the expression plasmids (see Example 15 for details).

TABLE 19

Sequences of the primers for the
construction of the fusion proteins
HBc-122, HBc-124, HBc-128 and HBc-129.

| Peptides | Primer sequences |
|---|---|
| HBc122F1 | 5′-TTT AGA TCT GGA GGA GGT GGT TCT GAG ACT |
| | CAG CTG ACT ACG GCG GGC CTG CGA CTT CTC-3′ |
| | (SEQ ID NO: 145) |
| HBc122F2 | 5′-GGC CTG CGA CTT CTC GGA GGA GGT GGT TCT |
| | GAG ACT CAG CTG ACT ACG GCG GGT CTT CGG-3′ |
| | (SEQ ID NO: 146) |
| HBc122F3 | 5′-ACG GCG GGT CTT CGG CTGCTT GGA TCC GTC |
| | GAC GGT GGT GGA GGT TCA GG-3′ |
| | (SEQ ID NO: 147) |
| HBc124F1 | 5′-TTT AGA TCT GGA GGA GGT GGT TCT CAG ACG |
| | CCG CTG ACT ATG GCT GCG CTG GAA CTG TTC-3′ |
| | (SEQ ID NO: 148) |
| HBc124F2 | 5′-GCG CTG GAA CTG TTC GGA GGA GGT GGT TCT |
| | CAG ACG CCG CTG ACT ATG GCT GCT CTT GAG-3′ |
| | (SEQ ID NO: 149) |
| HBc124F3 | 5′- ATG GCT GCT CTT GAG CTT TTT GGA TCC |
| | GTC GACGGTGGTGGAGGTTCAGG-3′ |
| | (SEQ ID NO: 150) |
| HBc128F1 | 5′-TTT AGA TCT GGA GGA GGT GGT TCT CAGACG |
| | CCT CTT ACG GAG ACG GCG CTA AAA TGG CAC-3′ |
| | (SEQ ID NO: 151) |
| HBc128F2 | 5′-GCG CTA AAA TGG CAC GGA GGA GGT GGT TCT |
| | CAG ACG CCT CTT ACG GAG ACG GCT TTG AAG-3′ |
| | (SEQ ID NO: 152) |
| HBc128F3 | 5′-GAG ACG GCT TTG AAG TGG CAT GGA TCC GTC |
| | GAC GGT GGT GGA GGT TCA GG-3′ |
| | (SEQ ID NO: 153) |
| HBc129F1 | 5′-TTT AGA TCT GGA GGA GGT GGT TCT CAG ACG |
| | CCT CTG ACT ATG GCG GCG CTG GAA TTG CTG-3′ |
| | (SEQ ID NO: 154) |
| HBc129F2 | 5′-GCG CTG GAA TTG CTG GGA GGA GGT GGT TCT |
| | CAG ACG CCT CTG ACT ATG GCG GCT CTT GAG-3′ |
| | (SEQ ID NO: 155) |
| HBc129F3 | 5′-ATG GCG GCT CTT GAG CTT CTT GGA TCC GTC |
| | GAC GGT GGT GGA GGT TCA GG-3′ |
| | (SEQ ID NO: 156) |
| HBcR | 5′-TT GAA TTC TTA AAC AAC ACT AGT TT-3′ |
| | (SEQ ID NO: 157) |

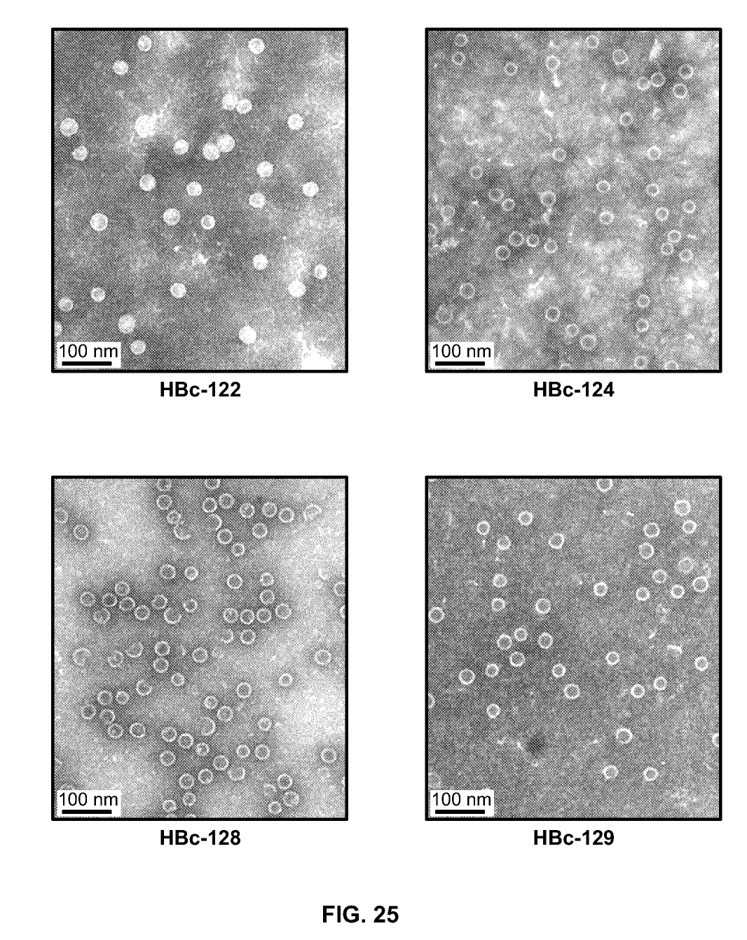

The method for the expression and purification of the fusion proteins HBc-122, HBc-124, HBc-128 and HBc-129 were the same as that for the fission proteins HBc-123 and HBc-125 (see Example 15 for details). The results of the expression and particle assembly of the fusion proteins of the 12aa peptides and the HBc are shown in Table 20 below. The electron microscopy pictures of the assembled particles are shown in FIG. 25.

TABLE 20
The expression of the fusion proteins of the 12aa peptides
and HBc and the formation of virus like particles.

| | Peptide | | | Particles |
|---|---|---|---|---|
| | Section | | Expression | Assembling |
| No. | Title | Production Form | Yield | Status |
| HBc-122 | 122 | Soluble | +++ | Good |
| HBc-D123 | 123 | Soluble | +++ | Good |
| HBc-T123 | 123 | Soluble | +++ | Ok |
| HBc-F123 | 123 | Soluble | +++ | Good |
| HBc-Q123 | 123 | Soluble | ++ | Good |
| HBc-124 | 124 | Soluble/Inclusion body | +++ | Good |
| HBc-D125 | 125 | Soluble | +++ | Good |
| HBc-T125 | 125 | Soluble/Inclusion body | ++ | Ok |
| HBc-F125 | 125 | Soluble/Inclusion body | ++ | Ok |
| HBc-Q125 | 125 | Soluble/Inclusion body | ++ | Ok |
| HBc-128 | 128 | Soluble | +++ | Good |
| HBc-129 | 129 | Soluble/Inclusion body | ++ | Good |

Using indirect ELISA to detect the activities of fusion proteins HBc-122, HBc-124, HBc-128 and HBc-129

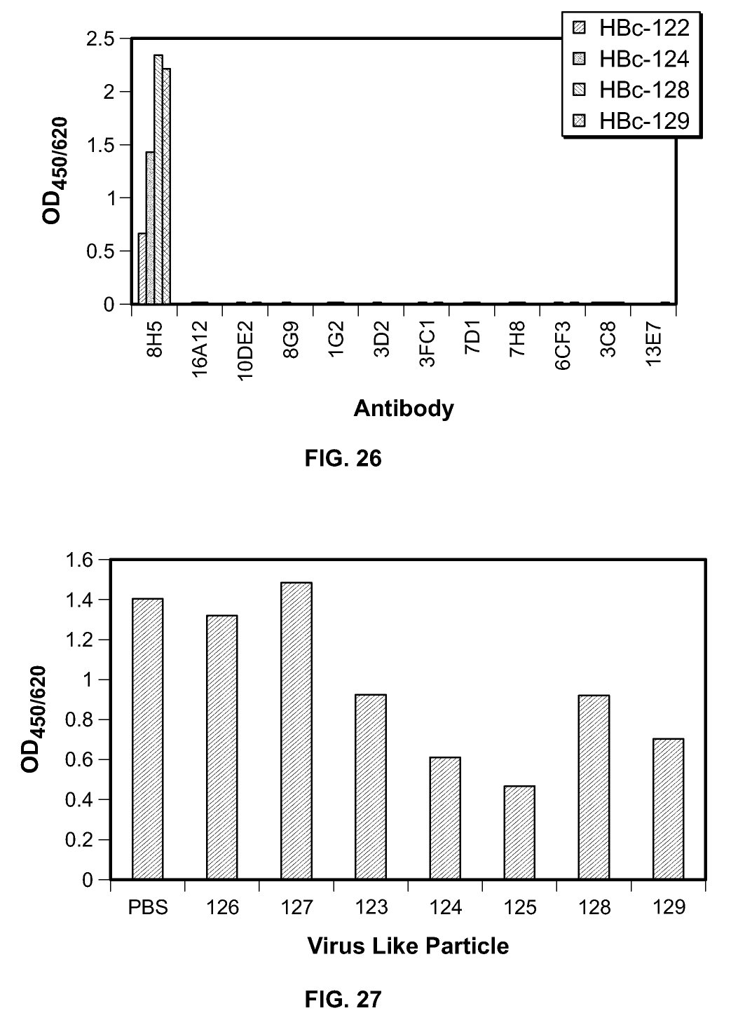

The fusion proteins HBc-122, HBc-124, HBc-128 and HBc-129 were separately imbedded onto 96-well plates at the concentration of 10 μg/ml at 37° C. for 2 hr. The plates were washed once, and treated with ED blocking buffer at 37° C. for 2 hr and then at 4° C. overnight to block non-specific binding sites. Next, different mAb were added to the plates at 100 μl per well, including a total of 12 mAb: 8H5, 8G9, 3C8, 1G2, 3D2, 3CF1, 7D1, 6CF3, 7H8, 10DE2, 13E7, 16A12. The 8H5 mAb was used to screen the 12aa peptides. The other 11 mAb were antibodies against the HA protein of avian influenza virus. The mAb were incubated in the plates at 37° C. for 1 hr. The plates were washed with PBST for 5 times. 100 μl GAM-HRP (1:10,000 dilution) was added to each well and incubated at 37° C. for 30 min. The plates were washed with PBST for 5 times. Coloring solution was added to the plates for 10 min for color development. Then stopping buffer was added to stop the color reaction. The intensity of the color was read with microplate reader. The results in FIG. 26 showed that fusion proteins HBc-122, HBc-124, HBc-128 and HBc-129 reacted only with 8H5, and did not react with any other mAb. The results indicated that fusion proteins HBc-122, HBc-124, HBc-128 and HBc-129 had very good binding specificity to 8H5 mAb.

### Example 20

### Competitive ELISA of Virus Like Particles Displaying 12aa Peptides and Avian Influenza Virus

Mouse 2F2 mAb that could specifically bind to H5N1 avian influenza virus was imbedded onto microplates at the concentration of 10 μg/ml. 1:40 diluted virus strain Ck/HK/Yu22/02 was added to the wells and incubated at 37° C. for 1 hr. After that, the solution in the wells was discarded. 10 μg of purified virus like particles and 1:1,000 diluted 8H5/HRP were added to the wells together and incubated at 37° C. for 30 min. 12aa peptides 126 and 127 could not bind to 8H5 mAb but was displayed on the virus like particles as negative controls. Additionally, PBS without virus like particles was also used as a negative control. The plates were washed with PBST for 5 times. Coloring solution was added to the plates for 10 min for color development. Then stopping buffer was added to stop the color reaction. The intensity of the color was read with microplate reader. The results in FIG. 27 showed that virus like particles assembled from fusion proteins HBc-123, HBc-124, HBc-125, HBc-128 or HBc-129 could each simulate some part of the antigen site binding to 8H5 mAb.

1. A monoclonal antibody that specifically binds to the hemagglutinin of avian influenza virus subtype H5 wherein said monoclonal antibody comprises a variable heavy chain selected from the group consisting of:
(i) a variable heavy chain comprising one or more of the CDRs having the amino acid sequences set forth in SEQ ID NOs: 28-30; and
(ii) variable heavy chain comprising one or more of the CDRs having the amino acid sequences set forth in SEQ ID NOs: 46-48.

2. The monoclonal antibody of claim 1 wherein said variable heavy chain comprising an amino acid sequence selected from the group consisting of SEQ ID NO:2 and SEQ ID NO:17.

3. The monoclonal antibody of claim 1 further comprises a variable light chain selected from the group consisting of:
(i) a variable light chain comprising one or more of the CDRs having the amino acid sequences set forth in SEQ ID NOs: 31-33; and
(ii) a variable light chain comprising one or more of the CDRs having the amino acid sequences set forth in SEQ ID NOs: 49-51.

4. (canceled)

5. (canceled)

6. The monoclonal antibody of claim 2 wherein said variable light chain comprises an amino acid sequence selected from the group consisting of SEQ ID NO:4 and SEQ ID NO:19.

7. (canceled)

8. The monoclonal antibody of claim 1 wherein said monoclonal antibody is a Fab, Fab′, F(ab)₂ or Fv.

9. The monoclonal antibody of claim 1 wherein said monoclonal antibody binds to the hemagglutinin of avian influenza virus subtype H5 with a K_{D} Of less than 1×10⁻⁵ M.

10. The monoclonal antibody of claim 9 wherein said monoclonal antibody binds to the hemagglutinin with a K_{D} of less than 1×10⁻⁶ M.

11. The monoclonal antibody of claim 1 wherein said monoclonal antibody comprises non-CDR regions that are derived from a species different from murine.

12. The monoclonal antibody of claim 11 wherein said non-CDR regions are from a human antibody.

13. The monoclonal antibody of claim 12 wherein said human non-CDR regions have one or more amino acid substitutions from a murine antibody.

14. The monoclonal antibody of claim 6 wherein said monoclonal antibody is a monoclonal antibody selected from the group consisting of:
(i) the monoclonal antibody produced by the hybridoma cell line 8H5 (Deposit No. CCTCC-C200607); and
(ii) the monoclonal antibody produced by the hybridoma cell line 4D1 (Deposit No. CCTCC-C200606).

15. (canceled)

16. A monoclonal antibody that specifically binds to the hemagglutinin of avian influenza virus subtype H5 wherein said monoclonal antibody is capable of blocking by at least 50% of the hemagglutinin binding activity of the monoclonal antibody of claim 1.

17. The monoclonal antibody of claim 16 wherein said monoclonal antibody is capable of blocking the hemagglutinin binding activity by at least 70%.

18. The monoclonal antibody of claim 17 wherein said monoclonal antibody is capable of blocking the hemagglutinin binding activity by at least 90%.

19. (canceled)

20. (canceled)

21. An isolated nucleic acid molecule encoding the antibody of claim 1, comprising a nucleic acid sequence encoding the heavy chain variable region selected from the group consisting of SEQ ID NO:1, and SEQ ID NO:16.

22-25. (canceled)

26. An isolated nucleic acid molecule encoding the antibody of claim 1, comprising a nucleic acid sequence encoding the light chain variable region selected from the group consisting of SEQ ID NO:3 and SEQ ID NO:18.

27. (canceled)

28. (canceled)

29. A method of detecting avian influenza virus subtype H5 in a sample comprising the steps of:
a) contacting said sample with a monoclonal antibody of claim 1; and
b) detecting the reaction of said monoclonal antibody with the virus.

30. The method of claim 29 wherein said monoclonal antibody is attached to a solid phase.

31. The method of claim 30 wherein said solid phase is selected from the group consisting of microtiter plates, magnetic particles, latex particles, and nitrocellulose membranes.

32. The method of claim 30 wherein said monoclonal antibody is attached to said solid phase in an orientation that increases the binding efficiency of the monoclonal antibody with the sample.

33. The method of claim 32 wherein said monoclonal antibody is attached to said solid phase through its constant regions.

34. The method of claim 29 wherein said reaction is detected by enzymatic color assay.

35. The method of claim 29 wherein said reaction is detected by fluorescence assay.

36. The method of claim 29 wherein said reaction is detected by chemiluminescence assay.

37. The method of claim 29 wherein said monoclonal antibody is a Fab, Fab′, F(ab)₂ or Fv.

38. The method of claim 29 wherein said sample is a biological sample from an avian or human subject.

39. A pharmaceutical composition comprising a pharmaceutically acceptable salt of the monoclonal antibody of claim 1.

40-43. (canceled)

44. A composition useful for detecting avian influenza virus in a sample comprising a monoclonal antibody of claim 1 attached to a solid phase substrate.

45-48. (canceled)

49. The composition of claim 44 wherein said solid phase substrate is a test strip.

50. The composition of claim 49 wherein said test strip has at least one testing area and one control area.

51. (canceled)

52. A device useful for detecting avian influenza virus in a sample comprising a solid phase substrate comprising a plurality of compartments, wherein one or more of said compartments are coated with the monoclonal antibody of claim 1.

53. The device of claim 52 wherein one or more of said compartment are coated with a binding agent different from said monoclonal antibody that specifically binds to the hemagglutinin of avian influenza virus subtype H5.

54. The device of claim 53 wherein said binding agent is an antibody that specifically binds to avian influenza virus subtype H1, H3, H7, or H9.

55. The device of claim 52 further comprising an automated detection device that can detect the binding of said monoclonal antibody to the hemagglutinin of avian influenza virus subtype H5.

56. A kit for detecting avian influenza virus in a sample comprising the monoclonal antibody of claim 1 attached to a solid phase substrate, and a detectably labeled secondary monoclonal antibody.

57. The kit of claim 56 wherein said secondary monoclonal antibody is capable of specifically binding avian influenza virus.

58. The kit of claim 56 wherein said secondary monoclonal antibody is capable of specifically binding to avian influenza virus hemagglutinin.

59. The kit of claim 56 further comprising control standards.

60-64. (canceled)


SEQUENCE LISTING (157 sequences)

SEQ ID NO: 1 - DNA, 363 bp, Mus musculus
caggttcagctgcagcagtctggagctgagctgatgaagcctggggcctcagtgaagatatcctgcaaggctactggctacactttcagtaactactggatagagtggataaagcagaggcctggacatggccttgagtggattggagagattttacctggaagcgatagaacaaactacaatgggaagttcaagggcaaggccacattcactgcagatacatcctccaacacagcccacatgcaactcagtagcctgacatctgaggactctgccgtctattactgtgcaaatagatacgacgggtattattttggtttggattactggggtcaaggaacctcagtcgccgtctcctcagcc

SEQ ID NO: 2 - protein, 121 aa, Mus musculus
QVQLQQSGAELMKPGASVKISCKATGYTFSNYWIEWIKQRPGHGLEWIGEILPGSDRTNYNGKFKGKATFTADTSSNTAHMQLSSLTSEDSAVYYCANRYDGYYFGLDYWGQGTSVAVSSA

SEQ ID NO: 3 - DNA, 321 bp, Mus musculus
gaaatcgtgctcacccagtctccagcaatcatgtctgcatctctaggggagaaggtcaccatgagctgcagggccagctcaagtgtaaatttcgtttactggtaccagcagaggtcagatgcctcccccaaactattgatttactattcatccaacctggctcctggagtcccacctcgcttcagtggcagtgggtctgggaactcttattctctcacaatcagcggcttggagggtgaagatgctgccacttattactgccagcactttactagttccccgtacacgttcggaggggggaccaacctggaaataaaacgg

SEQ ID NO: 4 - protein, 107 aa, Mus musculus
EIVLTQSPAIMSASLGEKVTMSCRASSSVNFVYWYQQRSDASPKLLIYYSSNLAPGVPPRFSGSGSGNSYSLTISGLEGEDAATYYCQHFTSSPYTFGGGTNLEIKR

SEQ ID NO: 5 - DNA, 351 bp, Mus musculus
cagatccagttggtgcagtctggacctgagctgaagaagcctggagagacagtcaagatctcctgcaaggcctctgggtacagcttcacaaactatggaatgaactgggtgaagcaggctccaggaaagggtctaaagtggatgggctggataaacacctacaccggagagccagcctatgctgatgacttcaagggacggtttgccttctctctggaaacctctgccagcactgcctatttgcagatcaacaacctcaaaaatgaggacacggctacatatttctgtgcaagatggaatagagatgctatggactactggggtcaaggaacctcggtcaccgtatctagc

SEQ ID NO: 6 - protein, 117 aa, Mus musculus
QIQLVQSGPELKKPGETVKISCKASGYSFTNYGMNWVKQAPGKGLKWMGWINTYTGEPAYADDFKGRFAFSLETSASTAYLQINNLKNEDTATYFCARWNRDAMDYWGQGTSVTVSS

SEQ ID NO: 7 - DNA, 339 bp, Mus musculus
gacattgtgctgacccaatctccagcttctttggctgtgtctcttgggcagagggccaccatatcctgcagagccagtgaaagtgttgatagttctgacaatagtcttatgcactggtaccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtattggggatcctccgtacacgttcggaggggggaccaagctggaaataaaacgg

SEQ ID NO: 8 - protein, 113 aa, Mus musculus
DIVLTQSPASLAVSLGQRATISCRASESVDSSDNSLMHWYQQKPGQPPKLLIYRASNLESGIPARFSGSGSRTDFTLTINPVEADDVATYYCQQSIGDPPYTFGGGTKLEIKR

SEQ ID NO: 9 - DNA, 363 bp, Mus musculus
caggtccaactgcagcagcctggggctgaacttgtgaagcctggggcttcagtgaagctgtcctgcaaggcttctggctacaccttcaccagctactggatgcactgggtgaagcagaggcctggacagggccttgagtggatcggagagattgatccttctgattcttatactaactacaatcagaagttcaagggcaaggccacattgactgtagacaaatcctccagcacagcctacatgcagctcagcagcctgacatctgaggactctgcggtctattactgtgcaagggggggtacaggagactttcactatgctatggactactggggtcaaggcacctcggtcaccgtatcatcg

SEQ ID NO: 10 - protein, 121 aa, Mus musculus
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCARGGTGDFHYAMDYWGQGTSVTVSS

SEQ ID NO: 11 - DNA, 324 bp, Mus musculus
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgttcagttcccgtacacgttcggagggggcaccaagctggaaatcaaacgg

SEQ ID NO: 12 - protein, 108 aa, Mus musculus
DILMTQSPSSMSVSLGDTVSITCHASQGISSNIGWLQQKPGKSFKGLIYHGTNLEDGVPSRFSGSGSGADYSLTISSLESEDFADYYCVQYVQFPYTFGGGTKLEIKR

SEQ ID NO: 13 - DNA, 21 bp, Mus musculus
catgggatgctgccggtgtat

SEQ ID NO: 14 - DNA, 21 bp, Mus musculus
aattctgggccttggctgacg

SEQ ID NO: 15 - DNA, 21 bp, Mus musculus
tggccgcctctgtcgaagaag

SEQ ID NO: 16 - DNA, 363 bp, Mus musculus
caggtccaactgcagcagcctggggctgagcttgtgaagcctggggcttcagtgaacctgtcctgtaaggcttctggctacaccttcaccagctactggatgcactgggtgaagcagaggcctggacaaggccttgagtggatcggagagattgatccttctgatagttttactacctacaatcaaaacttcaaagacagggccacattgactgtagacaaatcatccagcacagcctacatgcagctcagaagtctgacatctgaggactctgcggtctattactgtgccagggggggtccaggagactttcgctatgctatggattactggggccaaggcacctcggtcaccgtctcctca

SEQ ID NO: 17 - protein, 121 aa, Mus musculus
QVQLQQPGAELVKPGASVNLSCKASGYTFTSYWMHWVKQRPGQGLEWIGEIDPSDSFTTYNQNFKDRATLTVDKSSSTAYMQLRSLTSEDSAVYYCARGGPGDFRYAMDYWGQGTSVTVSS

SEQ ID NO: 18 - DNA, 327 bp, Mus musculus
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatccgaagactttgcagactattactgtgtacagtatgttcagtttccctacacgttcggaggggggaccaagctggaaataaaacgggct

SEQ ID NO: 19 - protein, 109 aa, Mus musculus
DILMTQSPSSMSVSLGDTVSITCHASQGISSNIGWLQQKPGKSFKGLIYHGTNLEDGVPSRFSGSGSGADYSLTISSLESEDFADYYCVQYVQFPYTFGGGTKLEIKRA

SEQ ID NO: 20 - DNA, 363 bp, Mus musculus
caggtccaactgcagcagtctggggctgagctggtgaggcctggggtctcagtgaagatttcctgcaagggttctggctacacattcactgattatgctatgcattgggtgaagcagagtcatgcaaagagtctagagtggattggacttattaatactgactatggtgatactacttacaaccagaagttcaagggcaaggccacaatgactgtagacaaatcctccaacacagcctatatggaacttgccagactgacatctgaggattctgccatctattactgtgcaagatcggactatgattactatttctgtggtatggactactggggtcaaggaaccacggtcaccgaatctcta

SEQ ID NO: 21 - protein, 121 aa, Mus musculus
QVQLQQSGAELVRPGVSVKISCKGSGYTFTDYAMHWVKQSHAKSLEWIGLINTDYGDTTYNQKFKGKATMTVDKSSNTAYMELARLTSEDSAIYYCARSDYDYYFCGMDYWGQGTTVTESL

SEQ ID NO: 22-23 - skipped (2 entries)

SEQ ID NO: 24 - DNA, 357 bp, Mus musculus
caggtgcagctgaaggagtcaggacctggcctggtggcgccctcacagcgcctgtccatcacatgcaccgtctcagggttctcattaaccggctatggtgtacactggattcgccagtctccaggaaagggtctggagtggctgggaatgatatgggctgagggaagaaccgactataattcagttctcaaatccagactgagcatcaataaggacaattccaggagccaagttttcttagaaatgaacagtctgcaaactgatgacacagccaggtactactgtgccagagaggtgattactacggaagcctggtacttcgatgtctggggccaaggaacctcggtcaccgaatct

SEQ ID NO: 25 - protein, 119 aa, Mus musculus
QVQLKESGPGLVAPSQRLSITCTVSGFSLTGYGVHWIRQSPGKGLEWLGMIWAEGRTDYNSVLKSRLSINKDNSRSQVFLEMNSLQTDDTARYYCAREVITTEAWYFDVWGQGTSVTES

SEQ ID NO: 26 - DNA, 324 bp, Mus musculus
gacattgtgatgactcagtctccagccaccctgtctgtgactccaggagatagagtctctctttcctgcagggccagccagagtattagcgactacttatactggtatcaacaaaaatcacatgagtctccaaggcttctcatcaaatatgcttcccaatccatctctgggatcccctccagattcagtggcagtggatcagggtcagatttcactctcactatcaacagtgtggaacctgaagatgttggaatgtattactgtcaaaatggtcacacctttccgctcacgttcggtgctggcaccaagctggaaatcaaacgg

SEQ ID NO: 27 - protein, 108 aa, Mus musculus
DIVMTQSPATLSVTPGDRVSLSCRASQSISDYLYWYQQKSHESPRLLIKYASQSISGIPSRFSGSGSGSDFTLTINSVEPEDVGMYYCQNGHTFPLTFGAGTKLEIKR

SEQ ID NO: 28 - protein, 8 aa, Mus musculus
GYTFSNYW

SEQ ID NO: 29 - protein, 8 aa, Mus musculus
ILPGSDRT

SEQ ID NO: 30 - protein, 13 aa, Mus musculus
ANRYDGYYFGLDY

SEQ ID NO: 31 - protein, 5 aa, Mus musculus
SSVNF

SEQ ID NO: 32 - protein, 3 aa, Mus musculus
YSS

SEQ ID NO: 33 - protein, 9 aa, Mus musculus
QHFTSSPYT

SEQ ID NO: 34 - protein, 8 aa, Mus musculus
GYSFTNYG

SEQ ID NO: 35 - protein, 8 aa, Mus musculus
INTHTGEP

SEQ ID NO: 36 - protein, 10 aa, Mus musculus
ARWNRDAMDY

SEQ ID NO: 37 - protein, 10 aa, Mus musculus
ESVDSSDNSL

SEQ ID NO: 38 - protein, 3 aa, Mus musculus
RAS

SEQ ID NO: 39 - protein, 10 aa, Mus musculus
QQSIGDPPYT

SEQ ID NO: 40 - protein, 8 aa, Mus musculus
GYTFTSYW

SEQ ID NO: 41 - protein, 8 aa, Mus musculus
IDPSDSYT

SEQ ID NO: 42 - protein, 14 aa, Mus musculus
ARGGTGDFHYAMDY

SEQ ID NO: 43 - protein, 6 aa, Mus musculus
QGISSN

SEQ ID NO: 44 - protein, 3 aa, Mus musculus
HGT

SEQ ID NO: 45 - protein, 8 aa, Mus musculus
QYVQFPYT

SEQ ID NO: 46 - protein, 8 aa, Mus musculus
GYTFTSYW

SEQ ID NO: 47 - protein, 8 aa, Mus musculus
IDPSDSFT

SEQ ID NO: 48 - protein, 14 aa, Mus musculus
ARGGPGDFRYAMDY

SEQ ID NO: 49 - protein, 6 aa, Mus musculus
QGISSN

SEQ ID NO: 50 - protein, 3 aa, Mus musculus
HGT

SEQ ID NO: 51 - protein, 9 aa, Mus musculus
VQYVQFPYT

SEQ ID NO: 52 - protein, 8 aa, Mus musculus
GYTFTDYA

SEQ ID NO: 53 - protein, 8 aa, Mus musculus
INTDYGDT

SEQ ID NO: 54 - protein, 14 aa, Mus musculus
ARSDYDYYFCGMDY

SEQ ID NO: 55-57 - skipped (3 entries)

SEQ ID NO: 58 - protein, 8 aa, Mus musculus
GFSLTGYG

SEQ ID NO: 59 - protein, 7 aa, Mus musculus
IWAEGRT

SEQ ID NO: 60 - protein, 14 aa, Mus musculus
AREVITTEAWYFDV

SEQ ID NO: 61 - protein, 6 aa, Mus musculus
QSISDY

SEQ ID NO: 62 - protein, 3 aa, Mus musculus
YAS

SEQ ID NO: 63 - protein, 9 aa, Mus musculus
QNGHTFPLT

SEQ ID NO: 64 - protein, 7 aa, Artificial Sequence
  Recombinant
HGMLPVY

SEQ ID NO: 65 - protein, 7 aa, Artificial Sequence
  Recombinant
PPSNYGR

SEQ ID NO: 66 - protein, 7 aa, Artificial Sequence
  Recombinant
PPSNFGK

SEQ ID NO: 67 - protein, 7 aa, Artificial Sequence
  Recombinant
GDPWFTS

SEQ ID NO: 68 - protein, 7 aa, Artificial Sequence
  Recombinant
NSGPWLT

SEQ ID NO: 69 - skipped

SEQ ID NO: 70 - protein, 7 aa, Artificial Sequence
  Recombinant
WPPLSKK

SEQ ID NO: 71 - protein, 7 aa, Artificial Sequence
  Recombinant
NTFRTPI

SEQ ID NO: 72 - protein, 7 aa, Artificial Sequence
  Recombinant
NTFRDPN

SEQ ID NO: 73 - protein, 7 aa, Artificial Sequence
  Recombinant
NPIWTKL

SEQ ID NO: 74 - protein, 12 aa, Artificial Sequence
  Recombinant
MEPVKKYPTRSP

SEQ ID NO: 75 - DNA, 36 bp, Artificial Sequence
  Recombinant
atggagccggtgaagaagtatccgacgcgttctcct

SEQ ID NO: 76 - protein, 12 aa, Artificial Sequence
  Recombinant
ETQLTTAGLRLL

SEQ ID NO: 77 - DNA, 36 bp, Artificial Sequence
  Recombinant
gagactcagctgactacggcgggtcttcggctgctt

SEQ ID NO: 78 - protein, 12 aa, Artificial Sequence
  Recombinant
ETPLTETALKWH

SEQ ID NO: 79 - DNA, 36 bp, Artificial Sequence
  Recombinant
gagacgcctcttacggagacggctttgaagtggcat

SEQ ID NO: 80 - protein, 12 aa, Artificial Sequence
  Recombinant
QTPLTMAALELF

SEQ ID NO: 81 - DNA, 36 bp, Artificial Sequence
  Recombinant
cagacgccgctgactatggctgctcttgagcttttt

SEQ ID NO: 82 - protein, 12 aa, Artificial Sequence
  Recombinant
DTPLTTAALRLV

SEQ ID NO: 83 - DNA, 36 bp, Artificial Sequence
  Recombinant
gatactccgctgacgacggcggctcttcggctggtt

SEQ ID NO: 84 - protein, 12 aa, Artificial Sequence
  Recombinant
TPLTLWALSGLR

SEQ ID NO: 85 - DNA, 36 bp, Artificial Sequence
  Recombinant
acgccgcttacgctttgggctctttctgggctgagg

SEQ ID NO: 86 - protein, 12 aa, Artificial Sequence
  Recombinant
QTPLTETALKWH

SEQ ID NO: 87 - DNA, 36 bp, Artificial Sequence
  Recombinant
cagacgcctcttacggagacggctttgaagtggcat

SEQ ID NO: 88 - protein, 12 aa, Artificial Sequence
  Recombinant
QTPLTMAALELL

SEQ ID NO: 89 - DNA, 36 bp, Artificial Sequence
  Recombinant
cagacgcctctgactatggcggctcttgagcttctt

SEQ ID NO: 90 - protein, 12 aa, Artificial Sequence
  Recombinant
HLQDGSPPSSPH

SEQ ID NO: 91 - DNA, 36 bp, Artificial Sequence; identical to SEQ ID NO: 89
  Recombinant

SEQ ID NO: 92 - protein, 12 aa, Artificial Sequence
  Recombinant
GHVTTLSLLSLR

SEQ ID NO: 93 - DNA, 36 bp, Artificial Sequence
  Recombinant
gggcatgtgacgactctttctcttctgtcgctgcgg

SEQ ID NO: 94 - protein, 12 aa, Artificial Sequence
  Recombinant
FPNFDWPLSPWT

SEQ ID NO: 95 - DNA, 36 bp, Recombinant
tttccgaattttgattggcctctgtctccgtggacg

SEQ ID NO: 96 - protein, 12 aa, Artificial Sequence
  Recombinant
ETPLTEPAFKRH

SEQ ID NO: 97 - DNA, 36 bp, Artificial Sequence
  Recombinant
gagacgcctcttacggagccggcttttaagcggcat

SEQ ID NO: 98 - DNA, 25 bp, Artificial Sequence
  Chemically synthesized
  misc_feature (5)..(5): n is a or g
  misc_feature (11)..(11): n is c or g
  misc_feature (13)..(14): n is g or t
  misc_feature (15)..(15): n is inosine
  misc_feature (16)..(16): n is a or g
  misc_feature (22)..(22): n is a or c
atggnatggancnnnntctttntct

SEQ ID NO: 99 - DNA, 29 bp, Artificial Sequence
  Chemically synthesized
  misc_feature (9)..(9): n is a or t
  misc_feature (12)..(12): n is a or g
  misc_feature (22)..(22): n is a or t
atggatttncangtgcagattntcagctt

SEQ ID NO: 100 - DNA, 29 bp, Artificial Sequence
  Chemically synthesized
  misc_feature (5)..(5): n is a, c or g
  misc_feature (10)..(10): n is c or g
  misc_feature (17)..(17): n is a or c
  misc_feature (23)..(23): n is c or t
atggnttggntgtggancttgcnattcct

SEQ ID NO: 101 - DNA, 27 bp, Artificial Sequence
  Chemically synthesized
  misc_feature (6)..(6): n is c or t
  misc_feature (9)..(9): n is c or t
  misc_feature (12)..(12): n is a, c or g
  misc_feature (15)..(15): n is a or g
atggtnctnatnttnctgctgctatgg

SEQ ID NO: 102 - DNA, 26 bp, Artificial Sequence
  Chemically synthesized
  misc_feature (4)..(4): n is a or g
  misc_feature (9)..(9): n is c or g
  misc_feature (11)..(11): n is c or g
  misc_feature (18): n is c or t
  misc_feature (19): n is a or t
  misc_feature (21)..(21): n is c or t
atgnaatgnanctgggtnntnctctt

SEQ ID NO: 103 - DNA, 25 bp, Artificial Sequence
  Chemically synthesized
  misc_feature (6)..(6): n is a or g
  misc_feature (10)..(10): n is a or t
  misc_feature (13)..(13): n is c or g
atggtntccncanctcagttccttg

SEQ ID NO: 104 - DNA, 26 bp, Artificial Sequence
  Chemically synthesized
  misc_feature (4)..(4): n is a or g
  misc_feature (9)..(9): n is c or g
  misc_feature (11)..(11): n is c or g
  misc_feature (18): n is c or t
  misc_feature (19): n is a or t
  misc_feature (21)..(21): n is c or t
atgnaatgnanctgggtnntnctctt

SEQ ID NO: 105 - DNA, 42 bp, Artificial Sequence
  Chemically synthesized
  misc_feature (17)..(17): n is a or g
  misc_feature (29)..(29): n is a or t
  misc_feature (32)..(32): n is c or t
  misc_feature (37)..(37): n is inosine
  misc_feature (38)..(38): n is a or t
actagtcgacatgaggncccctgctcagnttnttggnntctt

SEQ ID NO: 106 - DNA, 25 bp, Artificial Sequence
  Chemically synthesized
  misc_feature (5)..(5): n is a or g
  misc_feature (11)..(11): n is c or g
  misc_feature (13)..(14): n is g or t
  misc_feature (15)..(15): n is inosine
  misc_feature (16)..(16): n is a or g
  misc_feature (22)..(22): n is a or c
atggnatggancnnnntctttntct

SEQ ID NO: 107 - DNA, 30 bp, Artificial Sequence
  Chemically synthesized
  misc_feature (14)..(14): n is c or t
  misc_feature (16)..(16): n is a or g
  misc_feature (18)..(18): n is c, g or t
  misc_feature (23)..(23): n is c or t
  misc_feature (26)..(26): n is c or t
cgacatggctgtcntngngctgntcntctg

SEQ ID NO: 108 - DNA, 31 bp, Artificial Sequence
  Chemically synthesized
  misc_feature (10)..(10): n is c or t
  misc_feature (13)..(13): n is c or t
  misc_feature (16)..(16): n is a, c or g
cgacatggtnctnatntccttgctgttctgg

SEQ ID NO: 109 - DNA, 25 bp, Artificial Sequence
  Chemically synthesized
ttggatcccaggttcagctgcagca

SEQ ID NO: 110 - DNA, 59 bp, Artificial Sequence
  Chemically synthesized
gctaccaccccctccagatccgccacctcctgaggagacggtgactgaggttccttgac

SEQ ID NO: 111 - DNA, 51 bp, Artificial Sequence
  Chemically synthesized
atctggagggggtggtagcggtggaggcgggagtgaaatcgtgctcaccca

SEQ ID NO: 112 - DNA, 42 bp, Artificial Sequence
  Chemically synthesized
tttgtcgacccgttttatttccagcttggtcccccctccgaa

SEQ ID NO: 113 - DNA, 53 bp, Artificial Sequence
  Chemically synthesized
tcctgctactgattgtccctgcatatgtcctgtcccaggttcagctgcagcag

SEQ ID NO: 114 - DNA, 33 bp, Artificial Sequence
  Chemically synthesized
tttctcgagtgaggagacggtgactgaggttcc

SEQ ID NO: 115 - DNA, 50 bp, Artificial Sequence
  Chemically synthesized
tttggatccatgggaaggcttacttcttcattcctgctactgattgtccc

SEQ ID NO: 116 - DNA, 49 bp, Artificial Sequence
  Chemically synthesized
gctgctgctgtggcttacagatgcaagatgtgaaatcgtgctcacccag

SEQ ID NO: 117 - DNA, 39 bp, Artificial Sequence
  Chemically synthesized
tttctcgagccgttttatttccagcttggtcccccctcc

SEQ ID NO: 118 - DNA, 56 bp, Artificial Sequence
  Chemically synthesized
tttgaattcatgtctgtgccaactcaggtcctggggttgctgctgctgtggcttac

SEQ ID NO: 119 - DNA, 27 bp, Artificial Sequence
  Chemically synthesized
tttgaattccaggtccaactgcagcag

SEQ ID NO: 120 - DNA, 47 bp, Artificial Sequence
  Chemically synthesized
gctaccaccccctccagatccgccacctcccgatgatacggtgaccg

SEQ ID NO: 121 - DNA, 52 bp, Artificial Sequence
  Chemically synthesized
atctggagggggtggtagcggtggaggcgggagtgacatcctgatgacccaa

SEQ ID NO: 122 - DNA, 28 bp, Artificial Sequence
  Chemically synthesized
tttctcgagccgtttgatttccagcttg

SEQ ID NO: 123 - DNA, 53 bp, Artificial Sequence
  Chemically synthesized
tcctgctactgattgtccctgcatatgtcctgtcccaggtccaactgcagcag

SEQ ID NO: 124 - DNA, 43 bp, Artificial Sequence
  Chemically synthesized
tttctcgagcgatgatacggtgaccgaggtgccttgaccccag

SEQ ID NO: 125 - DNA, 50 bp, Artificial Sequence; identical to SEQ ID NO: 115
  Chemically synthesized

SEQ ID NO: 126 - DNA, 51 bp, Artificial Sequence
  Chemically synthesized
gctgctgctgtggcttacagatgcaagatgtgacatcctgatgacccaatc

SEQ ID NO: 127 - DNA, 27 bp, Artificial Sequence
  Chemically synthesized
tttctcgagagcccgttttatttccag

SEQ ID NO: 128 - DNA, 56 bp, Artificial Sequence; identical to SEQ ID NO: 118
  Chemially synthesized

SEQ ID NO: 129 - DNA, 49 bp, Artificial Sequence
  Chemically synthesized
ctctttttggtatcaacagcaacaggtgtccattcccaggtccaactgc

SEQ ID NO: 130 - DNA, 26 bp, Artificial Sequence
  Chemically synthesized
tttctcgagtgaggagacggtgaccg

SEQ ID NO: 131 - DNA, 50 bp, Artificial Sequence
  Chemically synthesized
tttggatccatgggatggtcctgtatcattctctttttggtatcaacagc

SEQ ID NO: 132 - DNA, 39 bp, Artificial Sequence
  Chemically synthesized
tttgaattcatgatggtccttgctcagtttcttgggttc

SEQ ID NO: 133 - DNA, 27 bp, Artificial Sequence
  Chemically synthesized
tttctcgagagcccgttttatttccag

SEQ ID NO: 134 - DNA, 27 bp, Artificial Sequence
  Chemically synthesized
tttttacatatgatagcgcttaccctg

SEQ ID NO: 135 - DNA, 50 bp, Artificial Sequence
  Chemically synthesized
gctaccaccaccaccagaaccaccaccaccgcgcggagggggggctaaac

SEQ ID NO: 136 - DNA, 59 bp, Artificial Sequence
  Chemically synthesized
tagaattcatgccacttcaaagccgtctccgtaagaggcgtctcgctacctccaccacc

SEQ ID NO: 137 - DNA, 27 bp, Artificial Sequence
  Chemically synthesized
tttttacatatgatagcgcttaccctg

SEQ ID NO: 138 - DNA, 51 bp, Artificial Sequence
  Chemically synthesized
gctaccaccaccaccagaaccaccaccaccgcgcggagggggggctaaaac

SEQ ID NO: 139 - DNA, 59 bp, Artificial Sequence
  Chemically synthesized
tagaattcaaccagccgaagagccgccgtcgtcagcggagtatcgctacctccaccacc

SEQ ID NO: 140 - DNA, 58 bp, Artificial Sequence
  Chemically synthesized
tttagatctggaggaggtggttctgagacgcctcttacggagacggctttgaagtggc

SEQ ID NO: 141 - DNA, 58 bp, Artificial Sequence
  Chemically synthesized
cggctttgaagtggcatggatccggtggcggatctctgcagggtggtggaggttcagg

SEQ ID NO: 142 - DNA, 58 bp, Artificial Sequence
  Chemically synthesized
tttagatctggaggaggtggttctgatactcccctgacgacggcggctcttcggctgg

SEQ ID NO: 143 - DNA, 58 bp, Artificial Sequence
  Chemically synthesized
cggctcttcggctggttggatccggtggcggatctctgcagggtggtggaggttcagg

SEQ ID NO: 144 - DNA, 25 bp, Artificial Sequence
  Chemically synthesized
ttgaattcttaaacaacagtagttt

SEQ ID NO: 145 - DNA, 60 bp, Artificial Sequence
  Chemically synthesized
tttagatctggaggaggtggttctgagactcagctgactacggcgggcctgcgacttctc

SEQ ID NO: 146 - DNA, 60 bp, Artificial Sequence
  Chemically synthesized
ggcctgcgacttctcggaggaggtggttctgagactcagctgactacggcgggtcttcgg

SEQ ID NO: 147 - DNA, 50 bp, Artificial Sequence
  Chemically synthesized
acggcgggtcttcggctgcttggatccgtcgacggtggtggaggttcagg

SEQ ID NO: 148 - DNA, 60 bp, Artificial Sequence
  Chemically synthesized
tttagatctggaggaggtggttctcagacgccgctgactatggctgcgctggaactgttc

SEQ ID NO: 149 - DNA, 60 bp, Artificial Sequence
  Chemically synthesized
gcgctggaactgttcggaggaggtggttctcagacgccgctgactatggctgctcttgag

SEQ ID NO: 150 - DNA, 50 bp, Artificial Sequence
  Chemically synthesized
atggctgctcttgagctttttggatccgtcgacggtggtggaggttcagg

SEQ ID NO: 151 - DNA, 60 bp, Artificial Sequence
  Chemically synthesized
tttagatctggaggaggtggttctcagacgcctcttacggagacggcgctaaaatggcac

SEQ ID NO: 152 - DNA, 60 bp, Artificial Sequence
  Chemically synthesized
gcgctaaaatggcacggaggaggtggttctcagacgcctcttacggagacggctttgaag

SEQ ID NO: 153 - DNA, 50 bp, Artificial Sequence
  Chemically synthesized
gagacggctttgaagtggcatggatccgtcgacggtggtggaggttcagg

SEQ ID NO: 154 - DNA, 60 bp, Artificial Sequence
  Chemically synthesized
tttagatctggaggaggtggttctcagacgcctctgactatggcggcgctggaattgctg

SEQ ID NO: 155 - DNA, 60 bp, Artificial Sequence
  Chemically synthesized
gcgctggaattgctgggaggaggtggttctcagacgcctctgactatggcggctcttgag

SEQ ID NO: 156 - DNA, 50 bp, Artificial Sequence
  Chemically synthesized
atggcggctcttgagcttcttggatccgtcgacggtggtggaggttcagg

SEQ ID NO: 157 - DNA, 25 bp, Artificial Sequence
  Chemically synthesized
ttgaattcttaaacaacagtagttt